SEQUENCE LISTING (330 sequences)

SEQ ID NO: 1 - DNA, 23 bp, synthetic construct
  source 1..23: mol_type = other DNA
ctgacagttagagccgatatcac

SEQ ID NO: 2 - DNA, 24 bp, synthetic construct
  source 1..24: mol_type = other DNA
caattcagtcttcagcaacttgag

SEQ ID NO: 3 - DNA, 18 bp, synthetic construct
  source 1..18: mol_type = other DNA
aacctgcagatacagctc

SEQ ID NO: 4 - DNA, 22 bp, synthetic construct
  source 1..22: mol_type = other DNA
ggtggctttaaaatgtcaggaa

SEQ ID NO: 5 - DNA, 19 bp, synthetic construct
  source 1..19: mol_type = other DNA
tgtcgccaagtttgatggt

SEQ ID NO: 6 - DNA, 19 bp, synthetic construct
  source 1..19: mol_type = other DNA
ctgtgtattcggccaaagc

SEQ ID NO: 7 - DNA, 23 bp, synthetic construct
  source 1..23: mol_type = other DNA
ctctgcatttttggacataggag

SEQ ID NO: 8 - DNA, 21 bp, synthetic construct
  source 1..21: mol_type = other DNA
gccttgcacttccattatgac

SEQ ID NO: 9 - DNA, 16 bp, synthetic construct
  source 1..16: mol_type = other DNA
gtactgtcattcacct

SEQ ID NO: 10 - DNA, 20 bp, synthetic construct
  source 1..20: mol_type = other DNA
gaggcctacattctgaacgc

SEQ ID NO: 11 - DNA, 23 bp, synthetic construct
  source 1..23: mol_type = other DNA
gtggttctttcttttgccttctc

SEQ ID NO: 12 - DNA, 15 bp, synthetic construct
  source 1..15: mol_type = other DNA
ctgcatcgtcattct

SEQ ID NO: 13 - DNA, 20 bp, synthetic construct
  source 1..20: mol_type = other DNA
gtttcatcctccctgtgctg

SEQ ID NO: 14 - DNA, 20 bp, synthetic construct
  source 1..20: mol_type = other DNA
gctccttgatcttccgcttc

SEQ ID NO: 15 - DNA, 14 bp, synthetic construct
  source 1..14: mol_type = other DNA
ctgcttttgttggt

SEQ ID NO: 16 - DNA, 18 bp, synthetic construct
  source 1..18: mol_type = other DNA
gatgccccacttgcagta

SEQ ID NO: 17 - DNA, 21 bp, synthetic construct
  source 1..21: mol_type = other DNA
cctcgtaaactggctaatggt

SEQ ID NO: 18 - DNA, 24 bp, synthetic construct
  source 1..24: mol_type = other DNA
acctaccccaatatatgaaggaaa

SEQ ID NO: 19 - DNA, 17 bp, synthetic construct
  source 1..17: mol_type = other DNA
ctgctgccacaaccagt

SEQ ID NO: 20 - DNA, 21 bp, synthetic construct
  source 1..21: mol_type = other DNA
ttcacatccatctggtacgtg

SEQ ID NO: 21 - DNA, 13 bp, synthetic construct
  source 1..13: mol_type = other DNA
ctgccgcaaattc

SEQ ID NO: 22 - DNA, 20 bp, synthetic construct
  source 1..20: mol_type = other DNA
gatccttatgccatcaccgt

SEQ ID NO: 23 - DNA, 22 bp, synthetic construct
  source 1..22: mol_type = other DNA
atcagcagagtcaatcagtgag

SEQ ID NO: 24 - DNA, 12 bp, synthetic construct
  source 1..12: mol_type = other DNA
cagcgttcccgg

SEQ ID NO: 25 - DNA, 21 bp, synthetic construct
  source 1..21: mol_type = other DNA
cctggatgctgacatttctga

SEQ ID NO: 26 - DNA, 20 bp, synthetic construct
  source 1..20: mol_type = other DNA
tcctccactcatctccaact

SEQ ID NO: 27 - DNA, 16 bp, synthetic construct
  source 1..16: mol_type = other DNA
atcacagagggagacc

SEQ ID NO: 28 - DNA, 24 bp, synthetic construct
  source 1..24: mol_type = other DNA
caatgtgataggtactctcagagg

SEQ ID NO: 29 - DNA, 20 bp, synthetic construct
  source 1..20: mol_type = other DNA
tgttccaaagctcctcacaa

SEQ ID NO: 30 - DNA, 16 bp, synthetic construct
  source 1..16: mol_type = other DNA
agctgctccactctga

SEQ ID NO: 31 - DNA, 18 bp, synthetic construct
  source 1..18: mol_type = other DNA
cagccagatgtgttgcca

SEQ ID NO: 32 - DNA, 21 bp, synthetic construct
  source 1..21: mol_type = other DNA
ctgtacggaatgcgtttcttg

SEQ ID NO: 33 - DNA, 13 bp, synthetic construct
  source 1..13: mol_type = other DNA
cagactccagcgg

SEQ ID NO: 34 - DNA, 21 bp, synthetic construct
  source 1..21: mol_type = other DNA
gctgtgtacagtcatggatgg

SEQ ID NO: 35 - DNA, 20 bp, synthetic construct
  source 1..20: mol_type = other DNA
gtcttcaggctcaaacaggt

SEQ ID NO: 36 - DNA, 18 bp, synthetic construct
  source 1..18: mol_type = other DNA
ttctttaggcaatgggca

SEQ ID NO: 37 - DNA, 17 bp, synthetic construct
  source 1..17: mol_type = other DNA
gtgtgctggacgctgga

SEQ ID NO: 38 - DNA, 25 bp, synthetic construct
  source 1..25: mol_type = other DNA
gtgataccttgaagcacaccattac

SEQ ID NO: 39 - DNA, 14 bp, synthetic construct
  source 1..14: mol_type = other DNA
gctcgggtgattct

SEQ ID NO: 40 - DNA, 22 bp, synthetic construct
  source 1..22: mol_type = other DNA
cacatttgagtgaagcttgtcg

SEQ ID NO: 41 - DNA, 21 bp, synthetic construct
  source 1..21: mol_type = other DNA
gcggatgtcaaacaagtcaag

SEQ ID NO: 42 - DNA, 15 bp, synthetic construct
  source 1..15: mol_type = other DNA
aatgaagccaccaca

SEQ ID NO: 43 - DNA, 21 bp, synthetic construct
  source 1..21: mol_type = other DNA
gaaggagaagatctgccagtg

SEQ ID NO: 44 - DNA, 19 bp, synthetic construct
  source 1..19: mol_type = other DNA
gctgactcctctgctcaag

SEQ ID NO: 45 - DNA, 15 bp, synthetic construct
  source 1..15: mol_type = other DNA
ccagagtcatcatgc

SEQ ID NO: 46 - DNA, 20 bp, synthetic construct
  source 1..20: mol_type = other DNA
ccagtagcctgattgtgcat

SEQ ID NO: 47 - DNA, 22 bp, synthetic construct
  source 1..22: mol_type = other DNA
tgtcagtgattctgttcaagga

SEQ ID NO: 48 - DNA, 19 bp, synthetic construct
  source 1..19: mol_type = other DNA
aagagggcattttggttgt

SEQ ID NO: 49 - DNA, 19 bp, synthetic construct
  source 1..19: mol_type = other DNA
actcttacaccacggctga

SEQ ID NO: 50 - DNA, 21 bp, synthetic construct
  source 1..21: mol_type = other DNA
ccatcaaggctctgtatccat

SEQ ID NO: 51 - DNA, 16 bp, synthetic construct
  source 1..16: mol_type = other DNA
aggatcactgtcagga

SEQ ID NO: 52 - DNA, 22 bp, synthetic construct
  source 1..22: mol_type = other DNA
ggatctgagcaggagaaatacc

SEQ ID NO: 53 - DNA, 24 bp, synthetic construct
  source 1..24: mol_type = other DNA
gaattcttcattcccttgaactga

SEQ ID NO: 54 - DNA, 20 bp, synthetic construct
  source 1..20: mol_type = other DNA
gaaaaagacaaattccaaag

SEQ ID NO: 55 - DNA, 23 bp, synthetic construct
  source 1..23: mol_type = other DNA
aagatggacaggtatgacaagtc

SEQ ID NO: 56 - DNA, 23 bp, synthetic construct
  source 1..23: mol_type = other DNA
actctttccacatagtcagatgg

SEQ ID NO: 57 - DNA, 19 bp, synthetic construct
  source 1..19: mol_type = other DNA
atttttaacccactcctcg

SEQ ID NO: 58 - DNA, 20 bp, synthetic construct
  source 1..20: mol_type = other DNA
tgtcctggctgttcattctg

SEQ ID NO: 59 - DNA, 20 bp, synthetic construct
  source 1..20: mol_type = other DNA
tggatccctatctcttgcca

SEQ ID NO: 60 - DNA, 13 bp, synthetic construct
  source 1..13: mol_type = other DNA
ctgtccatctcct

SEQ ID NO: 61 - DNA, 19 bp, synthetic construct
  source 1..19: mol_type = other DNA
ggcaattggtttgaggcaa

SEQ ID NO: 62 - DNA, 24 bp, synthetic construct
  source 1..24: mol_type = other DNA
ggactggataaatgtattcaagca

SEQ ID NO: 63 - DNA, 28 bp, synthetic construct
  source 1..28: mol_type = other DNA
tgcaagttatcaagaagttttgtaagtt

SEQ ID NO: 64 - DNA, 17 bp, synthetic construct
  source 1..17: mol_type = other DNA
ctggtggaggagaacgg

SEQ ID NO: 65 - DNA, 19 bp, synthetic construct
  source 1..19: mol_type = other DNA
ggtcgctggatgaaaggtt

SEQ ID NO: 66 - DNA, 11 bp, synthetic construct
  source 1..11: mol_type = other DNA
agcagctcgaa

SEQ ID NO: 67 - DNA, 19 bp, synthetic construct
  source 1..19: mol_type = other DNA
caggcatcgtcagtttcct

SEQ ID NO: 68 - DNA, 21 bp, synthetic construct
  source 1..21: mol_type = other DNA
acacaatggatctggtgctaa

SEQ ID NO: 69 - DNA, 16 bp, synthetic construct
  source 1..16: mol_type = other DNA
gataggtgctttgctg

SEQ ID NO: 70 - DNA, 22 bp, synthetic construct
  source 1..22: mol_type = other DNA
gaggagagaatcaacaaactgc

SEQ ID NO: 71 - DNA, 21 bp, synthetic construct
  source 1..21: mol_type = other DNA
aggttcaggtactccttccag

SEQ ID NO: 72 - DNA, 14 bp, synthetic construct
  source 1..14: mol_type = other DNA
attgaggcgcacat

SEQ ID NO: 73 - DNA, 21 bp, synthetic construct
  source 1..21: mol_type = other DNA
atacttctgcttggtgtaggc

SEQ ID NO: 74 - DNA, 22 bp, synthetic construct
  source 1..22: mol_type = other DNA
agccattgtactctttaaccca

SEQ ID NO: 75 - DNA, 15 bp, synthetic construct
  source 1..15: mol_type = other DNA
ggagactctgcgaga

SEQ ID NO: 76 - DNA, 23 bp, synthetic construct
  source 1..23: mol_type = other DNA
ccgagaattacgttcctacagtg

SEQ ID NO: 77 - DNA, 22 bp, synthetic construct
  source 1..22: mol_type = other DNA
gcggacattgtcatagtaagga

SEQ ID NO: 78 - DNA, 16 bp, synthetic construct
  source 1..16: mol_type = other DNA
tttgaaatcgacacac

SEQ ID NO: 79 - DNA, 18 bp, synthetic construct
  source 1..18: mol_type = other DNA
ctgccttgctctccttcc

SEQ ID NO: 80 - DNA, 22 bp, synthetic construct
  source 1..22: mol_type = other DNA
cttactcagcttgaacttgtcg

SEQ ID NO: 81 - DNA, 14 bp, synthetic construct
  source 1..14: mol_type = other DNA
gccacagatccatg

SEQ ID NO: 82 - DNA, 19 bp, synthetic construct
  source 1..19: mol_type = other DNA
acttccttgatccctgcca

SEQ ID NO: 83 - DNA, 22 bp, synthetic construct
  source 1..22: mol_type = other DNA
gtcttttcaaccatgtcctcca

SEQ ID NO: 84 - DNA, 15 bp, synthetic construct
  source 1..15: mol_type = other DNA
tgctgatggtcctca

SEQ ID NO: 85 - DNA, 17 bp, synthetic construct
  source 1..17: mol_type = other DNA
caatgccaccgaagcct

SEQ ID NO: 86 - DNA, 26 bp, synthetic construct
  source 1..26: mol_type = other DNA
cttttattttcatcctcagtgcaaac

SEQ ID NO: 87 - DNA, 18 bp, synthetic construct
  source 1..18: mol_type = other DNA
gagttgaaatctgaggcc

SEQ ID NO: 88 - DNA, 21 bp, synthetic construct
  source 1..21: mol_type = other DNA
cttgtcactttcgttcagcag

SEQ ID NO: 89 - DNA, 18 bp, synthetic construct
  source 1..18: mol_type = other DNA
cttcatggtgtgggctca

SEQ ID NO: 90 - DNA, 12 bp, synthetic construct
  source 1..12: mol_type = other DNA
tgcgggtactgg

SEQ ID NO: 91 - DNA, 21 bp, synthetic construct
  source 1..21: mol_type = other DNA
tccttggatgactctccctac

SEQ ID NO: 92 - DNA, 21 bp, synthetic construct
  source 1..21: mol_type = other DNA
agataccacctccctgaagaa

SEQ ID NO: 93 - DNA, 15 bp, synthetic construct
  source 1..15: mol_type = other DNA
aggagcgggatggag

SEQ ID NO: 94 - DNA, 21 bp, synthetic construct
  source 1..21: mol_type = other DNA
agaggtttaatgggctcacag

SEQ ID NO: 95 - DNA, 22 bp, synthetic construct
  source 1..22: mol_type = other DNA
ctctggtctgtcgtcatgtaag

SEQ ID NO: 96 - DNA, 15 bp, synthetic construct
  source 1..15: mol_type = other DNA
tcaccttctccacca

SEQ ID NO: 97 - DNA, 22 bp, synthetic construct
  source 1..22: mol_type = other DNA
cacaccaccatacctggataat

SEQ ID NO: 98 - DNA, 20 bp, synthetic construct
  source 1..20: mol_type = other DNA
tcacttgaggccaagagttc

SEQ ID NO: 99 - DNA, 15 bp, synthetic construct
  source 1..15: mol_type = other DNA
ggtccagccaagttc

SEQ ID NO: 100 - DNA, 22 bp, synthetic construct
  source 1..22: mol_type = other DNA
ctttcttcagggtctggtcatt

SEQ ID NO: 101 - DNA, 24 bp, synthetic construct
  source 1..24: mol_type = other DNA
cttgtcgtcttcggaaatgttatg

SEQ ID NO: 102 - DNA, 15 bp, synthetic construct
  source 1..15: mol_type = other DNA
tgactctgggagaaa

SEQ ID NO: 103 - DNA, 19 bp, synthetic construct
  source 1..19: mol_type = other DNA
gaggcaagtcagcctttct

SEQ ID NO: 104 - DNA, 20 bp, synthetic construct
  source 1..20: mol_type = other DNA
tgtccatcttgtcgtcttcg

SEQ ID NO: 105 - DNA, 15 bp, synthetic construct
  source 1..15: mol_type = other DNA
gatgactctgggaga

SEQ ID NO: 106 - DNA, 20 bp, synthetic construct
  source 1..20: mol_type = other DNA
gctcaccatgtgtgacttga

SEQ ID NO: 107 - DNA, 20 bp, synthetic construct
  source 1..20: mol_type = other DNA
tgggagagagacagcttgta

SEQ ID NO: 108 - DNA, 15 bp, synthetic construct
  source 1..15: mol_type = other DNA
gagagctgcatcagt

SEQ ID NO: 109 - DNA, 19 bp, synthetic construct
  source 1..19: mol_type = other DNA
gaaagtccacgctcaccat

SEQ ID NO: 110 - DNA, 18 bp, synthetic construct
  source 1..18: mol_type = other DNA
gcagccttgctctctagc

SEQ ID NO: 111 - DNA, 15 bp, synthetic construct
  source 1..15: mol_type = other DNA
gagagctgcatcagt

SEQ ID NO: 112 - DNA, 23 bp, synthetic construct
  source 1..23: mol_type = other DNA
ctctgcacaaactcttccatttc

SEQ ID NO: 113 - DNA, 20 bp, synthetic construct
  source 1..20: mol_type = other DNA
tttcctcgcccattcttacc

SEQ ID NO: 114 - DNA, 17 bp, synthetic construct
  source 1..17: mol_type = other DNA
ttacagtgaagtcctcc

SEQ ID NO: 115 - DNA, 21 bp, synthetic construct
  source 1..21: mol_type = other DNA
ggaaggagggaacagaaatcc

SEQ ID NO: 116 - DNA, 22 bp, synthetic construct
  source 1..22: mol_type = other DNA
gtgagctactggctgaactatt

SEQ ID NO: 117 - DNA, 19 bp, synthetic construct
  source 1..19: mol_type = other DNA
atttgagaagaatggtgga

SEQ ID NO: 118 - DNA, 20 bp, synthetic construct
  source 1..20: mol_type = other DNA
aggagatgtgctggattgtc

SEQ ID NO: 119 - DNA, 25 bp, synthetic construct
  source 1..25: mol_type = other DNA
tctgcatgaattatacattgaccac

SEQ ID NO: 120 - DNA, 14 bp, synthetic construct
  source 1..14: mol_type = other DNA
aactgaccacgctg

SEQ ID NO: 121 - DNA, 24 bp, synthetic construct
  source 1..24: mol_type = other DNA
actgcagagataagtttagctgac

SEQ ID NO: 122 - DNA, 23 bp, synthetic construct
  source 1..23: mol_type = other DNA
tcaccattttgcttacttccttg

SEQ ID NO: 123 - DNA, 16 bp, synthetic construct
  source 1..16: mol_type = other DNA
ttgttcaagaagccac

SEQ ID NO: 124 - DNA, 19 bp, synthetic construct
  source 1..19: mol_type = other DNA
catgttgcctacagcctct

SEQ ID NO: 125 - DNA, 23 bp, synthetic construct
  source 1..23: mol_type = other DNA
tctccaaacttcttcctcattcc

SEQ ID NO: 126 - DNA, 17 bp, synthetic construct
  source 1..17: mol_type = other DNA
atcagcaggttcatgca

SEQ ID NO: 127 - DNA, 22 bp, synthetic construct
  source 1..22: mol_type = other DNA
gaagatcatcctgtcagacgag

SEQ ID NO: 128 - DNA, 22 bp, synthetic construct
  source 1..22: mol_type = other DNA
cttccgagctagaacctgtatg

SEQ ID NO: 129 - DNA, 15 bp, synthetic construct
  source 1..15: mol_type = other DNA
tcaactcatttcggc

SEQ ID NO: 130 - DNA, 22 bp, synthetic construct
  source 1..22: mol_type = other DNA
gatcagacagtcattcgcaaag

SEQ ID NO: 131 - DNA, 21 bp, synthetic construct
  source 1..21: mol_type = other DNA
gacaatcttccagggactgag

SEQ ID NO: 132 - DNA, 15 bp, synthetic construct
  source 1..15: mol_type = other DNA
gttcagagggttctt

SEQ ID NO: 133 - DNA, 18 bp, synthetic construct
  source 1..18: mol_type = other DNA
cccaacccaggcatgatg

SEQ ID NO: 134 - DNA, 20 bp, synthetic construct
  source 1..20: mol_type = other DNA
tcaatgagaagcaccttggc

SEQ ID NO: 135 - DNA, 12 bp, synthetic construct
  source 1..12: mol_type = other DNA
tccagcagagct

SEQ ID NO: 136 - DNA, 21 bp, synthetic construct
  source 1..21: mol_type = other DNA
tgctggaatggacaagaactc

SEQ ID NO: 137 - DNA, 21 bp, synthetic construct
  source 1..21: mol_type = other DNA
gctcatggagattgaactggt

SEQ ID NO: 138 - DNA, 16 bp, synthetic construct
  source 1..16: mol_type = other DNA
ccttttggctgtatct

SEQ ID NO: 139 - DNA, 21 bp, synthetic construct
  source 1..21: mol_type = other DNA
cttactggcgttttctcatgc

SEQ ID NO: 140 - DNA, 23 bp, synthetic construct
  source 1..23: mol_type = other DNA
ccaactcttgtagaggtctcaag

SEQ ID NO: 141 - DNA, 17 bp, synthetic construct
  source 1..17: mol_type = other DNA
gcccacttttcctaggt

SEQ ID NO: 142 - DNA, 21 bp, synthetic construct
  source 1..21: mol_type = other DNA
ggaccagggaaagataaagcc

SEQ ID NO: 143 - DNA, 21 bp, synthetic construct
  source 1..21: mol_type = other DNA
gcaaggtagattcgtgacaga

SEQ ID NO: 144 - DNA, 15 bp, synthetic construct
  source 1..15: mol_type = other DNA
aatcccaaccacaaa

SEQ ID NO: 145 - DNA, 22 bp, synthetic construct
  source 1..22: mol_type = other DNA
cattttgtggttgggattctgg

SEQ ID NO: 146 - DNA, 18 bp, synthetic construct
  source 1..18: mol_type = other DNA
gatgctgtggatgtggct

SEQ ID NO: 147 - DNA, 17 bp, synthetic construct
  source 1..17: mol_type = other DNA
atctaccttgctgctca

SEQ ID NO: 148 - DNA, 21 bp, synthetic construct
  source 1..21: mol_type = other DNA
gccttgcacttccattatgac

SEQ ID NO: 149 - DNA, 23 bp, synthetic construct
  source 1..23: mol_type = other DNA
ctctgcatttttggacataggag

SEQ ID NO: 150 - DNA, 13 bp, synthetic construct
  source 1..13: mol_type = other DNA
gaggtcctgaggc

SEQ ID NO: 151 - DNA, 22 bp, synthetic construct
  source 1..22: mol_type = other DNA
atgtggagtttacagtgtctgg

SEQ ID NO: 152 - DNA, 20 bp, synthetic construct
  source 1..20: mol_type = other DNA
agcttctcactgagtgttgc

SEQ ID NO: 153 - DNA, 15 bp, synthetic construct
  source 1..15: mol_type = other DNA
cagccaagtgtaacc

SEQ ID NO: 154 - DNA, 17 bp, synthetic construct
  source 1..17: mol_type = other DNA
acggctcccatcctcct

SEQ ID NO: 155 - DNA, 22 bp, synthetic construct
  source 1..22: mol_type = other DNA
ccactatgtcaccatgtacctg

SEQ ID NO: 156 - DNA, 12 bp, synthetic construct
  source 1..12: mol_type = other DNA
agaaccagcagc

SEQ ID NO: 157 - DNA, 20 bp, synthetic construct
  source 1..20: mol_type = other DNA
tgctctgacaacccttatgc

SEQ ID NO: 158 - DNA, 21 bp, synthetic construct
  source 1..21: mol_type = other DNA
ggctgaggatcactttgtaga

SEQ ID NO: 159 - DNA, 15 bp, synthetic construct
  source 1..15: mol_type = other DNA
gtctttgctgacatt

SEQ ID NO: 160 - DNA, 19 bp, synthetic construct
  source 1..19: mol_type = other DNA
catcagcaggaccagtagc

SEQ ID NO: 161 - DNA, 19 bp, synthetic construct
  source 1..19: mol_type = other DNA
tgtctgtgctccctgatct

SEQ ID NO: 162 - DNA, 15 bp, synthetic construct
  source 1..15: mol_type = other DNA
agtaccaggactgct

SEQ ID NO: 163 - DNA, 22 bp, synthetic construct
  source 1..22: mol_type = other DNA
tcatttggacgtactgacttgg

SEQ ID NO: 164 - DNA, 19 bp, synthetic construct
  source 1..19: mol_type = other DNA
cttgctccagctcctgttc

SEQ ID NO: 165 - DNA, 13 bp, synthetic construct
  source 1..13: mol_type = other DNA
ggttctctgcctg

SEQ ID NO: 166 - DNA, 21 bp, synthetic construct
  source 1..21: mol_type = other DNA
ggtgtttggtctaggatggag

SEQ ID NO: 167 - DNA, 20 bp, synthetic construct
  source 1..20: mol_type = other DNA
actgggtttgacttcgtagc

SEQ ID NO: 168 - DNA, 16 bp, synthetic construct
  source 1..16: mol_type = other DNA
atgctgtattttgcac

SEQ ID NO: 169 - DNA, 22 bp, synthetic construct
  source 1..22: mol_type = other DNA
gtgactctcctgacatccttag

SEQ ID NO: 170 - DNA, 21 bp, synthetic construct
  source 1..21: mol_type = other DNA
ccatctcatttcgtcctccaa

SEQ ID NO: 171 - DNA, 17 bp, synthetic construct
  source 1..17: mol_type = other DNA
acagacataggcaaagt

SEQ ID NO: 172 - DNA, 24 bp, synthetic construct
  source 1..24: mol_type = other DNA
actctgaaaaactttggactgatg

SEQ ID NO: 173 - DNA, 24 bp, synthetic construct
  source 1..24: mol_type = other DNA
tctagcaatcaacagatgagttct

SEQ ID NO: 174 - DNA, 12 bp, synthetic construct
  source 1..12: mol_type = other DNA
gagccaaacgcc

SEQ ID NO: 175 - DNA, 19 bp, synthetic construct
  source 1..19: mol_type = other DNA
agctccttccagtccgaat

SEQ ID NO: 176 - DNA, 21 bp, synthetic construct
  source 1..21: mol_type = other DNA
gtctgctcatcaatcacctca

SEQ ID NO: 177 - DNA, 17 bp, synthetic construct
  source 1..17: mol_type = other DNA
atacccattgtcatcgc

SEQ ID NO: 178 - DNA, 23 bp, synthetic construct
  source 1..23: mol_type = other DNA
ggacagagagaacaaggatgaac

SEQ ID NO: 179 - DNA, 23 bp, synthetic construct
  source 1..23: mol_type = other DNA
tgtgggagaatataggtggattg

SEQ ID NO: 180 - DNA, 15 bp, synthetic construct
  source 1..15: mol_type = other DNA
ctgtgctggacaatg

SEQ ID NO: 181 - DNA, 23 bp, synthetic construct
  source 1..23: mol_type = other DNA
gctttgacatcagtagaccagag

SEQ ID NO: 182 - DNA, 20 bp, synthetic construct
  source 1..20: mol_type = other DNA
ctgtccgcagatcagacttg

SEQ ID NO: 183 - DNA, 19 bp, synthetic construct
  source 1..19: mol_type = other DNA
tcaaaaagtggaaaggtga

SEQ ID NO: 184 - DNA, 19 bp, synthetic construct
  source 1..19: mol_type = other DNA
cctggaaaatggcctcctt

SEQ ID NO: 185 - DNA, 21 bp, synthetic construct
  source 1..21: mol_type = other DNA
cattgcctacaggaagtctgg

SEQ ID NO: 186 - DNA, 17 bp, synthetic construct
  source 1..17: mol_type = other DNA
tatgccaagagtgtgag

SEQ ID NO: 187 - DNA, 20 bp, synthetic construct
  source 1..20: mol_type = other DNA
ccagaggtaaaggtgccaac

SEQ ID NO: 188 - DNA, 22 bp, synthetic construct
  source 1..22: mol_type = other DNA
tcccagataactgtcatgaagc

SEQ ID NO: 189 - DNA, 19 bp, synthetic construct
  source 1..19: mol_type = other DNA
gcagagtaactacaaaggc

SEQ ID NO: 190 - DNA, 22 bp, synthetic construct
  source 1..22: mol_type = other DNA
cctcaaatacatcaagcacagc

SEQ ID NO: 191 - DNA, 18 bp, synthetic construct
  source 1..18: mol_type = other DNA
ggaagccttcaccagcaa

SEQ ID NO: 192 - DNA, 13 bp, synthetic construct
  source 1..13: mol_type = other DNA
cccaggtggtcca

SEQ ID NO: 193 - DNA, 20 bp, synthetic construct
  source 1..20: mol_type = other DNA
ttgcaggcttcacatacctt

SEQ ID NO: 194 - DNA, 17 bp, synthetic construct
  source 1..17: mol_type = other DNA
gcccgacatgcttgagt

SEQ ID NO: 195 - DNA, 14 bp, synthetic construct
  source 1..14: mol_type = other DNA
ggacaacgaccttt

SEQ ID NO: 196 - DNA, 20 bp, synthetic construct
  source 1..20: mol_type = other DNA
ctcttgcagccattcctctt

SEQ ID NO: 197 - DNA, 20 bp, synthetic construct
  source 1..20: mol_type = other DNA
cccttaccccagtcacttct

SEQ ID NO: 198 - DNA, 18 bp, synthetic construct
  source 1..18: mol_type = other DNA
cctcttctctcctcccct

SEQ ID NO: 199 - DNA, 25 bp, synthetic construct
  source 1..25: mol_type = other DNA
tggacagaagacatactcataaagg

SEQ ID NO: 200 - DNA, 18 bp, synthetic construct
  source 1..18: mol_type = other DNA
ggtgccagcatgaatccc

SEQ ID NO: 201 - DNA, 17 bp, synthetic construct
  source 1..17: mol_type = other DNA
tggacctgcagttatca

SEQ ID NO: 202 - DNA, 21 bp, synthetic construct
  source 1..21: mol_type = other DNA
atcttccctccattctgcttc

SEQ ID NO: 203 - DNA, 22 bp, synthetic construct
  source 1..22: mol_type = other DNA
cagttcccactcactttctcag

SEQ ID NO: 204 - DNA, 12 bp, synthetic construct
  source 1..12: mol_type = other DNA
gccaccccactc

SEQ ID NO: 205 - DNA, 19 bp, synthetic construct
  source 1..19: mol_type = other DNA
ttttgcaccagtctcgctt

SEQ ID NO: 206 - DNA, 19 bp, synthetic construct
  source 1..19: mol_type = other DNA
gccgcactgacagtatgag

SEQ ID NO: 207 - DNA, 11 bp, synthetic construct
  source 1..11: mol_type = other DNA
tgggagccctg

SEQ ID NO: 208 - DNA, 18 bp, synthetic construct
  source 1..18: mol_type = other DNA
cgactgcgagtgataccg

SEQ ID NO: 209 - DNA, 17 bp, synthetic construct
  source 1..17: mol_type = other DNA
ctctccacgcactccct

SEQ ID NO: 210 - DNA, 13 bp, synthetic construct
  source 1..13: mol_type = other DNA
aacagccacaacg

SEQ ID NO: 211 - DNA, 18 bp, synthetic construct
  source 1..18: mol_type = other DNA
tgccgctcatgttcatgc

SEQ ID NO: 212 - DNA, 20 bp, synthetic construct
  source 1..20: mol_type = other DNA
caggacaccatgaggaacag

SEQ ID NO: 213 - DNA, 10 bp, synthetic construct
  source 1..10: mol_type = other DNA
tcccgcttca

SEQ ID NO: 214 - DNA, 22 bp, synthetic construct
  source 1..22: mol_type = other DNA
aagatgtcagacactgagaacg

SEQ ID NO: 215 - DNA, 17 bp, synthetic construct
  source 1..17: mol_type = other DNA
cgaagcccgatgtggtc

SEQ ID NO: 216 - DNA, 16 bp, synthetic construct
  source 1..16: mol_type = other DNA
agtccaaagcacacga

SEQ ID NO: 217 - DNA, 20 bp, synthetic construct
  source 1..20: mol_type = other DNA
aggagatgtgctggattgtc

SEQ ID NO: 218 - DNA, 25 bp, synthetic construct
  source 1..25: mol_type = other DNA
tctgcatgaattatacattgaccac

SEQ ID NO: 219 - DNA, 14 bp, synthetic construct
  source 1..14: mol_type = other DNA
aactgaccacgctg

SEQ ID NO: 220 - DNA, 22 bp, synthetic construct
  source 1..22: mol_type = other DNA
atgtggagtttacagtgtctgg

SEQ ID NO: 221 - DNA, 20 bp, synthetic construct
  source 1..20: mol_type = other DNA
agcttctcactgagtgttgc

SEQ ID NO: 222 - DNA, 15 bp, synthetic construct
  source 1..15: mol_type = other DNA
cagccaagtgtaacc

SEQ ID NO: 223 - DNA, 21 bp, synthetic construct
  source 1..21: mol_type = other DNA
gagatctgcttgaatgtgctg

SEQ ID NO: 224 - DNA, 22 bp, synthetic construct
  source 1..22: mol_type = other DNA
caacagaggtttttcacagcat

SEQ ID NO: 225 - DNA, 15 bp, synthetic construct
  source 1..15: mol_type = other DNA
tggcaaggtccgccc

SEQ ID NO: 226 - DNA, 21 bp, synthetic construct
  source 1..21: mol_type = other DNA
catggtaggctgagatgcttt

SEQ ID NO: 227 - DNA, 22 bp, synthetic construct
  source 1..22: mol_type = other DNA
gacgataaggagacctgctttg

SEQ ID NO: 228 - DNA, 15 bp, synthetic construct
  source 1..15: mol_type = other DNA
tgcaagtcaagctgc

SEQ ID NO: 229 - DNA, 22 bp, synthetic construct
  source 1..22: mol_type = other DNA
gcgcattctggaatttgtactc

SEQ ID NO: 230 - DNA, 21 bp, synthetic construct
  source 1..21: mol_type = other DNA
gctatgccaaagtgttcgatg

SEQ ID NO: 231 - DNA, 15 bp, synthetic construct
  source 1..15: mol_type = other DNA
ggaagagcctcagaa

SEQ ID NO: 232 - DNA, 22 bp, synthetic construct
  source 1..22: mol_type = other DNA
tgatggatattctctggatggc

SEQ ID NO: 233 - DNA, 25 bp, synthetic construct
  source 1..25: mol_type = other DNA
cctgaatctttactctctctccttg

SEQ ID NO: 234 - DNA, 25 bp, synthetic construct
  source 1..25: mol_type = other DNA
cagtttggtacattctatttcttcc

SEQ ID NO: 235 - DNA, 21 bp, synthetic construct
  source 1..21: mol_type = other DNA
gcacttcaagttcaccatcac

SEQ ID NO: 236 - DNA, 22 bp, synthetic construct
  source 1..22: mol_type = other DNA
accagtttattgtcaccttcca

SEQ ID NO: 237 - DNA, 17 bp, synthetic construct
  source 1..17: mol_type = other DNA
cttgactttctcccctg

SEQ ID NO: 238 - DNA, 28 bp, synthetic construct
  source 1..28: mol_type = other DNA
acatctattattgctactattgtgtgtt

SEQ ID NO: 239 - DNA, 20 bp, synthetic construct
  source 1..20: mol_type = other DNA
tgggagcctcttctctcttc

SEQ ID NO: 240 - DNA, 15 bp, synthetic construct
  source 1..15: mol_type = other DNA
attgtcgttgacacc

SEQ ID NO: 241 - DNA, 24 bp, synthetic construct
  source 1..24: mol_type = other DNA
ttcatttgataagcacacagtctg

SEQ ID NO: 242 - DNA, 22 bp, synthetic construct
  source 1..22: mol_type = other DNA
accttgaacatggcatagtctg

SEQ ID NO: 243 - DNA, 16 bp, synthetic construct
  source 1..16: mol_type = other DNA
agtcttccagttccac

SEQ ID NO: 244 - DNA, 19 bp, synthetic construct
  source 1..19: mol_type = other DNA
aatcagctccgcttccttg

SEQ ID NO: 245 - DNA, 21 bp, synthetic construct
  source 1..21: mol_type = other DNA
tgcttatctcgttgtccttcg

SEQ ID NO: 246 - DNA, 14 bp, synthetic construct
  source 1..14: mol_type = other DNA
catcatccacatcc

SEQ ID NO: 247 - DNA, 22 bp, synthetic construct
  source 1..22: mol_type = other DNA
cagaagcgcagaagattgtaag

SEQ ID NO: 248 - DNA, 20 bp, synthetic construct
  source 1..20: mol_type = other DNA
tctttctgatctgccatcgc

SEQ ID NO: 249 - DNA, 13 bp, synthetic construct
  source 1..13: mol_type = other DNA
gtccacaacggtt

SEQ ID NO: 250 - DNA, 18 bp, synthetic construct
  source 1..18: mol_type = other DNA
agaagcgagtccgactgt

SEQ ID NO: 251 - DNA, 19 bp, synthetic construct
  source 1..19: mol_type = other DNA
cactgcacaccatctcaca

SEQ ID NO: 252 - DNA, 11 bp, synthetic construct
  source 1..11: mol_type = other DNA
gccccaggacg

SEQ ID NO: 253 - DNA, 22 bp, synthetic construct
  source 1..22: mol_type = other DNA
cagatggaggaggaagattctg

SEQ ID NO: 254 - DNA, 22 bp, synthetic construct
  source 1..22: mol_type = other DNA
gtatactgcctggagttctctg

SEQ ID NO: 255 - DNA, 22 bp, synthetic construct
  source 1..22: mol_type = other DNA
ctggttcaggtctccattacag

SEQ ID NO: 256 - DNA, 20 bp, synthetic construct
  source 1..20: mol_type = other DNA
gctgtgactctgagcaagta

SEQ ID NO: 257 - DNA, 23 bp, synthetic construct
  source 1..23: mol_type = other DNA
tgtcctcctcaatctggtttatg

SEQ ID NO: 258 - DNA, 21 bp, synthetic construct
  source 1..21: mol_type = other DNA
gacagaagggcttggagattt

SEQ ID NO: 259 - DNA, 19 bp, synthetic construct
  source 1..19: mol_type = other DNA
cggtggcgttgtagaagat

SEQ ID NO: 260 - DNA, 19 bp, synthetic construct
  source 1..19: mol_type = other DNA
agaagatctctgcctccga

SEQ ID NO: 261 - DNA, 22 bp, synthetic construct
  source 1..22: mol_type = other DNA
caagatagttgtggtgggagac

SEQ ID NO: 262 - DNA, 21 bp, synthetic construct
  source 1..21: mol_type = other DNA
agggtctctggtctactgatg

SEQ ID NO: 263 - DNA, 19 bp, synthetic construct
  source 1..19: mol_type = other DNA
tttggatttaccgcttggg

SEQ ID NO: 264 - DNA, 18 bp, synthetic construct
  source 1..18: mol_type = other DNA
gactccagtgtgggagag

SEQ ID NO: 265 - DNA, 22 bp, synthetic construct
  source 1..22: mol_type = other DNA
gggcccacatataaatcctcac

SEQ ID NO: 266 - DNA, 21 bp, synthetic construct
  source 1..21: mol_type = other DNA
ctgctggtcactgttctcatc

SEQ ID NO: 267 - DNA, 17 bp, synthetic construct
  source 1..17: mol_type = other DNA
cttcgcggtgtggtgaa

SEQ ID NO: 268 - DNA, 18 bp, synthetic construct
  source 1..18: mol_type = other DNA
gctgtgtctcccgtcaaa

SEQ ID NO: 269 - DNA, 19 bp, synthetic construct
  source 1..19: mol_type = other DNA
ctgggacacattgccttct

SEQ ID NO: 270 - DNA, 23 bp, synthetic construct
  source 1..23: mol_type = other DNA
ccaccatgcattctttcaattct

SEQ ID NO: 271 - DNA, 22 bp, synthetic construct
  source 1..22: mol_type = other DNA
aaacccagtttgaggagatgag

SEQ ID NO: 272 - DNA, 22 bp, synthetic construct
  source 1..22: mol_type = other DNA
ccctgccaatatcttgggtaat

SEQ ID NO: 273 - DNA, 20 bp, synthetic construct
  source 1..20: mol_type = other DNA
acagcaacttccttgatccc

SEQ ID NO: 274 - DNA, 20 bp, synthetic construct
  source 1..20: mol_type = other DNA
gcggcatcactgtctatgaa

SEQ ID NO: 275 - DNA, 21 bp, synthetic construct
  source 1..21: mol_type = other DNA
ccttgccttctcttaggcttt

SEQ ID NO: 276 - DNA, 19 bp, synthetic construct
  source 1..19: mol_type = other DNA
agcagtggtttcagcatca

SEQ ID NO: 277 - DNA, 27 bp, synthetic construct
  source 1..27: mol_type = other DNA
cagataactctcattcagtattcttgg

SEQ ID NO: 278 - DNA, 24 bp, synthetic construct
  source 1..24: mol_type = other DNA
ctctaatgtagcttgacctcatct

SEQ ID NO: 279 - DNA, 21 bp, synthetic construct
  source 1..21: mol_type = other DNA
gagaagttggacaagattggg

SEQ ID NO: 280 - DNA, 25 bp, synthetic construct
  source 1..25: mol_type = other DNA
gctgagaagttctgtgaattcttta

SEQ ID NO: 281 - DNA, 23 bp, synthetic construct
  source 1..23: mol_type = other DNA
gtttcctcaaccagtcacataga

SEQ ID NO: 282 - DNA, 23 bp, synthetic construct
  source 1..23: mol_type = other DNA
agttgtctagcagtttccacata

SEQ ID NO: 283 - DNA, 20 bp, synthetic construct
  source 1..20: mol_type = other DNA
gggaaagcctgtctgaagtg

SEQ ID NO: 284 - DNA, 21 bp, synthetic construct
  source 1..21: mol_type = other DNA
tcgtagcctccagggtaatag

SEQ ID NO: 285 - DNA, 23 bp, synthetic construct
  source 1..23: mol_type = other DNA
gttacaggtctcctatctacagc

SEQ ID NO: 286 - DNA, 18 bp, synthetic construct
  source 1..18: mol_type = other DNA
gctcagcctctctggaag

SEQ ID NO: 287 - DNA, 22 bp, synthetic construct
  source 1..22: mol_type = other DNA
ctctcttaccctgattcggatg

SEQ ID NO: 288 - DNA, 17 bp, synthetic construct
  source 1..17: mol_type = other DNA
ggcgtctgcctgtgatt

SEQ ID NO: 289 - DNA, 20 bp, synthetic construct
  source 1..20: mol_type = other DNA
cctgagttctggtgccaaag

SEQ ID NO: 290 - DNA, 18 bp, synthetic construct
  source 1..18: mol_type = other DNA
gggcatgagcagcttcaa

SEQ ID NO: 291 - DNA, 19 bp, synthetic construct
  source 1..19: mol_type = other DNA
ccactggcttggtggattt

SEQ ID NO: 292 - DNA, 21 bp, synthetic construct
  source 1..21: mol_type = other DNA
tcaacagaaatgcccagagtt

SEQ ID NO: 293 - DNA, 18 bp, synthetic construct
  source 1..18: mol_type = other DNA
cttctccagctgggcatt

SEQ ID NO: 294 - DNA, 17 bp, synthetic construct
  source 1..17: mol_type = other DNA
tgctgtggcagcagatg

SEQ ID NO: 295 - DNA, 17 bp, synthetic construct
  source 1..17: mol_type = other DNA
cggtcatgtccgccttc

SEQ ID NO: 296 - DNA, 22 bp, synthetic construct
  source 1..22: mol_type = other DNA
gcgtttccattatgtcgttgtc

SEQ ID NO: 297 - DNA, 19 bp, synthetic construct
  source 1..19: mol_type = other DNA
ccctccttctaggatagcg

SEQ ID NO: 298 - DNA, 17 bp, synthetic construct
  source 1..17: mol_type = other DNA
aacccggaatgggtgat

SEQ ID NO: 299 - DNA, 20 bp, synthetic construct
  source 1..20: mol_type = other DNA
aaacggactgatgtcactgg

SEQ ID NO: 300 - DNA, 25 bp, synthetic construct
  source 1..25: mol_type = other DNA
tggacagaagacatactcataaagg

SEQ ID NO: 301 - DNA, 21 bp, synthetic construct
  source 1..21: mol_type = other DNA
cccactgcttcaggaaacata

SEQ ID NO: 302 - DNA, 22 bp, synthetic construct
  source 1..22: mol_type = other DNA
gtcagacatcttccctccattc

SEQ ID NO: 303 - DNA, 18 bp, synthetic construct
  source 1..18: mol_type = other DNA
gccgcactgacagtatga

SEQ ID NO: 304 - DNA, 20 bp, synthetic construct
  source 1..20: mol_type = other DNA
cagaaggagcaggactgaaa

SEQ ID NO: 305 - DNA, 18 bp, synthetic construct
  source 1..18: mol_type = other DNA
agcggcgcctcttatatc

SEQ ID NO: 306 - DNA, 22 bp, synthetic construct
  source 1..22: mol_type = other DNA
gcgttgaaagagaagacaaact

SEQ ID NO: 307 - DNA, 18 bp, synthetic construct
  source 1..18: mol_type = other DNA
ctactgcaacggcaacct

SEQ ID NO: 308 - DNA, 18 bp, synthetic construct
  source 1..18: mol_type = other DNA
gggccatgttcttgctca

SEQ ID NO: 309 - DNA, 19 bp, synthetic construct
  source 1..19: mol_type = other DNA
cagactcgctcgctcattt

SEQ ID NO: 310 - DNA, 19 bp, synthetic construct
  source 1..19: mol_type = other DNA
cctccatgcccactttctt

SEQ ID NO: 311 - DNA, 21 bp, synthetic construct
  source 1..21: mol_type = other DNA
cccaagtcagtacgtccaaat

SEQ ID NO: 312 - DNA, 24 bp, synthetic construct
  source 1..24: mol_type = other DNA
gcctaattcccgaataacatcaac

SEQ ID NO: 313 - DNA, 22 bp, synthetic construct
  source 1..22: mol_type = other DNA
gctttaaagaaagtgtttgctg

SEQ ID NO: 314 - DNA, 21 bp, synthetic construct
  source 1..21: mol_type = other DNA
ctgtatctgcaggttcgtaag

SEQ ID NO: 315 - DNA, 18 bp, synthetic construct
  source 1..18: mol_type = other DNA
aagttccccgtgtgcatc

SEQ ID NO: 316 - DNA, 18 bp, synthetic construct
  source 1..18: mol_type = other DNA
ctcagcctcctcgatgaa

SEQ ID NO: 317 - DNA, 19 bp, synthetic construct
  source 1..19: mol_type = other DNA
gtgaggaggcaaggttctg

SEQ ID NO: 318 - DNA, 19 bp, synthetic construct
  source 1..19: mol_type = other DNA
ggctccagagagggtagtt

SEQ ID NO: 319 - DNA, 19 bp, synthetic construct
  source 1..19: mol_type = other DNA
tttggatttaccgcttggg

SEQ ID NO: 320 - DNA, 18 bp, synthetic construct
  source 1..18: mol_type = other DNA
gactccagtgtgggagag

SEQ ID NO: 321 - DNA, 17 bp, synthetic construct
  source 1..17: mol_type = other DNA
cttcgcggtgtggtgaa

SEQ ID NO: 322 - DNA, 18 bp, synthetic construct
  source 1..18: mol_type = other DNA
gctgtgtctcccgtcaaa

SEQ ID NO: 323 - DNA, 21 bp, synthetic construct
  source 1..21: mol_type = other DNA
atcctgctgtatcacatcatg

SEQ ID NO: 324 - DNA, 21 bp, synthetic construct
  source 1..21: mol_type = other DNA
ctgacaggtttcaaagaacct

SEQ ID NO: 325 - DNA, 18 bp, synthetic construct
  source 1..18: mol_type = other DNA
ccagtgcctttggttgct

SEQ ID NO: 326 - DNA, 19 bp, synthetic construct
  source 1..19: mol_type = other DNA
caagagccagatgggcaag

SEQ ID NO: 327 - DNA, 20 bp, synthetic construct
  source 1..20: mol_type = other DNA
tgccaagagaagatgctcac

SEQ ID NO: 328 - DNA, 22 bp, synthetic construct
  source 1..22: mol_type = other DNA
cattgagtgccaacatgaagac

SEQ ID NO: 329 - DNA, 20 bp, synthetic construct
  source 1..20: mol_type = other DNA
aagaagctgccaatagggat

SEQ ID NO: 330 - DNA, 18 bp, synthetic construct
  source 1..18: mol_type = other DNA
tgtccagagaggtggatg

(19) United States
(12) Patent Application Publication
(10) Pub. No.: US 2024/0132969 A1
(43) Pub. Date: Apr. 25, 2024

(54) Digital Analysis of Circulating Tumor Cells in Blood Samples

(71) Applicant: The General Hospital Corporation, Boston, MA (US)

(72) Inventors: Daniel A. Haber, Chestnut Hill, MA (US); Ravi Kapur, Sharon, MA (US); Mehmet Toner, Charlestown, MA (US); Shyamala Maheswaran, Lexington, MA (US); Xin Hong, Malden, MA (US); David Tomoaki Miyamoto, Wellesley, MA (US); Tanya Todorova, Malden, MA (US); Sarah Javaid, Cambridge, MA (US)

(21) Appl. No.: 18/396,156

(22) Filed: Dec. 26, 2023

Related U.S. Application Data
(63) Continuation of application No. 17/065,889, filed on Oct. 8, 2020, now Pat. No. 11,898,209.
Application No. 17/065,889 is a continuation of application No. 15/560,324, filed on Sep. 21, 2017, now abandoned.
(60) Provisional application No. 62/219,339, filed on Sep. 16, 2015.
(60) Provisional application No. 62/137,891, filed on Mar. 25, 2015.
(60) Provisional application No. 62/253,619, filed on Nov. 10, 2015.

(51) Int. Cl. C12Q 1/6886; C12Q 1/686

(52) U.S. Cl. CPC C12Q 1/6886; C12Q 1/686; C12Q 2600/118; C12Q 2600/158; C12Q 2600/16

(57) ABSTRACT

This disclosure relates to new assay methods for analysis of circulating tumor cells (CTCs) in blood samples for detection, e.g., early detection, and/or monitoring of disease, e.g., cancer. The methods provide ultra-high sensitivity and specificity, and include the use of microfluidic isolation of CTCs and digital detection of RNA derived from the CTCs.

### CROSS-REFERENCE TO RELATED APPLICATIONS

This application is a continuation of U.S. application Ser. No. 17/065,889, filed on Oct. 8, 2020, which is a continuation of U.S. application Ser. No. 15/560,324, filed on Sep. 21, 2017, which is a 371 U.S. National of PCT Application No. PCT/US2016/024367, filed on Mar. 25, 2016, which claims priority from U.S. Provisional Application Ser. No. 62/253,619, filed on Nov. 10, 2015, U.S. Provisional Application Ser. No. 62/219,339, filed on Sep. 16, 2015, and U.S. Provisional Application Ser. No. 62/137,891, filed on Mar. 25, 2015, the contents of which are incorporated herein by reference in their entireties.

### SEQUENCE LISTING

This application contains a Sequence Listing that has been submitted electronically as an XML file named 28970-0854003_SL_ST26.xml. The XML file, created on Dec. 5, 2023, is 291,395 bytes in size. The material in the XML file is hereby incorporated by reference in its entirety.

### TECHNICAL FIELD

This invention relates to blood sampling techniques, and more particularly to methods and systems for detecting and analyzing cells in blood samples.

### BACKGROUND

The ability to detect the presence of rare circulating tumor cells (CTCs) using a simple blood test, or “liquid biopsy,” has the potential to greatly enhance the monitoring of epithelial cancers, providing instant sampling of tumor cell numbers, genetic composition, and drug response parameters, without requiring invasive tumor biopsies. Thus, the detection of CTCs for early cancer detection has the potential to revolutionize the treatment of cancer, enabling the diagnosis of invasive cancer at a stage before it has metastasized, when curative treatment is expected.

However, CTCs are very rare, and identifying, visualizing, and scoring these tumor cells admixed with normal blood components remains a significant challenge, even after partial purification with known microfluidic devices or similar technologies. For example, per milliliter of whole blood, there are only 1-10 CTCs amongst more than 5 billion red blood cells (RBCs) and more than 5 million white blood cells (WBCs)(Plaks et al., “Cancer Circulating Tumor Cells,” Science, 341:1186; 2013). In addition, antibody staining of tumor cells is highly variable, due to high heterogeneity among cancer cells, even within an individual patient, as well as the poor physical condition of many tumor cells that circulate in the bloodstream, many of which have begun to undergo programmed cell death or anoikis. In addition, accurate scoring of antibody-stained tumor cells requires differentiation from large numbers of contaminating white blood cells, some of which bind to antibody reagents non-specifically. As such, only a subset of candidate tumor cells can be robustly identified by antibody staining, and as many as half of patients tested have no detectable cells, despite having widely metastatic cancer.

Thus, current protocols for imaging CTCs are seeking higher and higher levels of purity in the isolation of CTCs, especially from other nucleated blood cells, such as white blood cells (WBCs).

### SUMMARY

The present disclosure relates to methods, uses, and systems to obtain the highest possible sensitivity of data relating to rare CTCs in standard blood samples, while avoiding the need for extremely high levels of purity of the CTCs. In particular, the new methods do not need the CTCs to be completely isolated from contaminating WBCs, and instead can reliably detect as few as one CTC in products containing, e.g., up to 10,000 WBCs or more. The new assay methods and systems combine (1) an isolation system that can consistently obtain CTCs as intact, whole cells (with high quality ribonucleic acid (RNA)) from blood with (2) a droplet-based digital polymerase chain reaction (PCR) assay focused on ribonucleic acid RNA markers of specific cancer lineages for each tumor type that are absent in blood of healthy patients.

When combined as described herein, these two concepts provide a CTC digital droplet PCR assay method (“CTC ddPCR”) or simply stated a “digital-CTC” assay (“d-CTC”). In some embodiments, the isolation system is a microfluidic system, such as a negative depletion microfluidic system (e.g., a so-called “CTC-Chip,” that uses negative depletion of hematopoietic cells, e.g., red blood cells (RBCs), WBCs, and platelets, to reveal untagged non-hematopoietic cells such as CTCs in a blood sample).

In general, the disclosure relates to methods for early detection of cancer with ultra-high sensitivity and specificity, wherein the methods include the use of microfluidic isolation of circulating tumor cells (CTCs) and digital detection of RNA derived from the CTCs. In some embodiments, the CTC-derived RNA can be converted into cDNA and encapsulated into individual droplets for amplification in the presence of reporter groups that are configured to bind specifically to cDNA from CTCs and not to cDNA from other cells. The droplets positive for reporter groups can be counted to assess the presence of disease, e.g., various types of cancer.

In another aspect, the disclosure relates to methods of analyzing circulating tumor cells (CTCs) in a blood sample. The methods include or consist of isolating from the blood sample a product comprising CTCs and other cells present in blood; isolating ribonucleic acid (RNA) molecules from the product; generating cDNA molecules in solution from the isolated RNA; encapsulating cDNA molecules in individual droplets; amplifying cDNA molecules within each of the droplets in the presence of reporter groups configured to bind specifically to cDNA from CTCs and not to cDNA from other cells; detecting droplets that contain the reporter groups as an indicator of the presence of cDNA molecules from CTCs in the droplets; and analyzing CTCs in the detected droplets.

The methods described herein can further include reducing a volume of the product before isolating RNA and/or removing contaminants from the cDNA-containing solution before encapsulating the cDNA molecules.

In various implementations of the new methods, generating cDNA molecules from the isolated RNA can include conducting reverse transcription (RT) polymerase chain reaction (PCR) of the isolated RNA molecules and/or amplifying cDNA molecules within each of the droplets can include conducting PCR in each droplet. In the new methods, encapsulating individual cDNA molecules and PCR reagents in individual droplets can include forming at least 1000 droplets of a non-aqueous liquid, such as one or more fluorocarbons, hydrofluorocarbons, mineral oils, silicone oils, and hydrocarbon oils and/or one or more surfactants. Each droplet can contain, on average, one target cDNA molecule obtained from a CTC. In some embodiments, the reporter groups can be or include a fluorescent label.

The new methods can include removing contaminants from the cDNA-containing solution by use of Solid Phase Reversible Immobilization (SPRI), e.g., immobilizing cDNA in the solution, e.g., with magnetic beads that are configured to specifically bind to the cDNA; removing contaminants from the solution; and eluting purified cDNA.

In various implementations, the methods described herein include using probes and primers in amplifying the cDNA molecules within each of the droplets that correspond to one or more genes selected from the list of cancer-selective genes in Table 1 herein. For example, the selected genes can include prostate cancer-selective genes, e.g., any one or more of AGR2, FOLH1, HOXDB13, KLK2, KLK3, SCHLAP1/SET4, SCHLAP1/SET5, AMACR, AR variants, UGT2B15/SET1, UGT2B15/SET5, and STEAP2 (as can be easily determined from Table 1). In another example, any one or more of ALDH1A3, CDH11, EGFR, FAT1, MET, PKP3, RND3, S100A2, and STEAP2 are selective for pancreatic cancer. Similar lists can be generated for the other types of cancers listed in Table 1.

In other examples, the selected genes include any one or more of the breast cancer-selective genes listed in Table 1. In other examples, the selected genes include genes selective for one or more of lung, liver, prostate, pancreatic, and melanoma cancer. For example, a multiplexed assay can include 2, 3, 4, 5, 6, 7, 8, 9, 10, 11, 12 or even all of the selected genes that are listed in Table 1 as being selective for a particular type of cancer, e.g., breast cancer, lung cancer, prostate cancer, pancreatic 12 cancer, liver cancer, and melanoma. Typically a group of primers and probes for 5 to cancer-selective genes from Table 1 are used for a particular type of cancer. Other specific combinations of selected genes (markers for those genes) are described in the Examples below.

The methods can also include using one or more genes selective for two or more, three or more, four or more, or five or more different types of cancer. For example, the genes can be selective for breast cancer and lung cancer; breast cancer, lung cancer, and liver cancer; breast cancer, lung cancer, and pancreatic cancer; breast cancer, lung cancer, and prostate cancer; breast cancer, liver cancer, and melanoma; breast cancer, lung cancer, and melanoma; breast cancer, lung cancer, liver cancer, and prostate cancer; breast cancer, lung cancer, liver cancer, and melanoma; breast cancer, lung cancer, liver cancer, and pancreatic cancer; breast cancer, lung cancer, prostate cancer, and pancreatic cancer; breast cancer, lung cancer, liver cancer, melanoma, and pancreatic cancer; or breast cancer, lung cancer, liver cancer, melanoma, pancreatic cancer, and prostate cancer.

In the methods described herein, the CTCs can arise from metastatic or primary/localized cancers. In the new methods, the step of analyzing the CTCs in the detected droplets cam include monitoring CTCs from a blood sample from a patient, e.g., with a known cancer, e.g., over time, and testing and/or imaging the CTCs (e.g., using standard techniques) to provide a prognosis for the patient. In other embodiments, the step of analyzing the CTCs in the detected droplets can include testing and/or imaging the CTCs (e.g., using standard techniques) from a blood sample from a patient to provide an indication of a response by the CTCs to a therapeutic intervention.

In other embodiments, the step of analyzing the CTCs in the detected droplets includes determining a number or level of CTCs per unit volume of a blood sample from a patient to provide a measure of tumor burden in the patient. The methods can then further include using the measure of tumor burden in the patient to select a therapy or can further include determining the measure of tumor burden in the patient at a second time point to monitor the tumor burden over time, e.g., in response to a therapeutic intervention, e.g., for dynamic monitoring of tumor burden.

The methods and assays described herein can be used to amplify and detect CTCs in a wide variety of diagnostic, prognostic, and theranostic methods.

As used herein, the phrase “circulating tumor cells” (CTCs) refers to cancer cells derived from solid tumors (non-hematogenous cancers) that are present in very rare numbers in the blood stream of patients (e.g., about 1 CTC in about 10,000,000 WBCs in whole blood). CTCs can arise from both metastatic as well as primary/localized cancers.

As used herein, a “product” means a group of isolated rare cells and other contaminating blood cells, e.g., red blood cells, white blood cells (e.g., leukocytes), e.g., in some sort of liquid, e.g., a buffer, such as a pluronic buffer, that arise from processing in the methods described herein, e.g., using the systems described herein. A typical product may contain only about one to ten CTCs admixed with 500 to 2,500 or more WBCs, e.g., one to ten CTCs in a mixture of 1000 to 2000 WBCs. However, the limit of detection of the present methods can be about 1 CTC in 10,000 WBC. Thus, while the present methods can achieve a level of purity of about 1 CTC in 500 WBCs, the present methods do not require highly purified CTCs, as is required in some known methods of CTC analysis.

As used herein a Solid Phase Reversible Immobilization (SPRI) cleanup is a technique using coated magnetic beads to perform size selection on cDNA created from Reverse Transcription (RT)-PCR of a product. In the new assay methods described herein this accomplishes the two-fold purpose of (a) selecting only the cDNA of the correct size, and (b) removing harsh lysis detergents incompatible with the stability of the droplets.

The polymerase chain reaction (PCR) is a process of amplification of known DNA fragments by serial annealing and re-annealing of small oligonucleotide primers, resulting in a detectable molecular signal.

Reverse Transcription (RT)-PCR refers to the use of reverse transcription to generate a complementary c-DNA molecule from an RNA template, thereby enabling the DNA polymerase chain reaction to operate on RNA. An important aspect of the new methods disclosed herein is the availability of high quality RNA from whole cell CTCs that are not lysed or treated in such a way that might destroy or degrade the RNA.

As used herein, “positive droplets” are lipid-encapsulated molecules in which a PCR reaction performed with tagged primers allows visualization of the PCR amplified product. Thus, a droplet that contained a single template cDNA molecule of a particular targeted gene can become visible using fluorescence microscopy, while an “empty” or “negative” droplet is one that contains no targeted cDNA.

The new methods and systems provide numerous advantages and benefits. For example, the current methods and systems provide results that are far more accurate and robust than either of the prior known systems when used alone. By breaking down the signal from a single CTC into hundreds or thousands of brightly fluorescent droplets, each derived from a single cDNA molecule, the new digital-CTC assays enable dramatic signal amplification. Given the strict criteria in selecting and optimizing the biomarker genes described herein, the background signal from normal blood cells is negligible in d-CTC. Thus, d-CTC enables greatly amplified signal from patients with advanced cancer (nearly 100% of patients with prostate, lung, breast, and liver cancers). Not only is the fraction of patients with a positive score significantly increased, but the high level of signal enables dynamic measurements as tumor load declines following cancer therapy. In addition, the signal amplification permits detection of CTC-derived signatures even in patients with a very low tumor burden (something that is not readily accomplished with CTC cell imaging), thus enabling significantly earlier detection of cancer.

In sum, this novel microfluidics platform provides a streamlined, ultrahigh-throughput, rapid (e.g., 3 hours per run), and extremely high sensitivity method of enriching, detecting, and analyzing CTCs in patient blood samples. The platform provides rich, clinically actionable information, and with further optimization may enable early detection of cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a graph showing cDNA dilutions prepared from total RNA of LNCaP prostate cancer cells, mixed with leukocytes and analyzed by droplet PCR using two different prostate primer sets. The results represent several purities and show good response of positive droplet number across this range.

FIG. 1B is a graph of manually isolated LNCaP cells spiked into healthy donor (HD) blood samples, run through the CTC-iChip, and subjected to droplet RT-PCR (KLK3 primer set). The results show excellent sensitivity down to low numbers of target cells.

FIG. 1C is a graph that shows the analysis of blood samples from healthy controls, patients with localized (resectable) prostate cancer and metastatic prostate cancer, processed through the CTC-iChip, subjected to RT-PCR and droplet analysis using three prostate-specific and one epithelial-specific biomarkers (KLK3, AMACR, FOLH1, EpCAM). The results are shown for the total number of droplets/ml for all four markers combined.

FIG. 2 is a signal intensity plot that shows KLK3 positive droplets derived from LNCAP prostate cancer cells spiked into blood and recovered using the CTC-iChip.

FIG. 3 is a bar graph that shows the minimal variation between experimental replicates and the retention of signal after sample processing through the CTC-iChip and shows increased detection sensitivity using the new assays described herein.

FIG. 4 is a signal intensity plot that shows the absence of four different cancer-specific marker-positive droplets in healthy donors using the new CTC digital droplet PCR assay methods described here (“CTC ddPCR” assay or simply “d-CTC” assay).

FIG. 5 is a signal intensity plot that shows a d-CTC assay multiplexed for four different lineage specific transcripts to detect prostate cancer cell lines spiked into blood.

FIGS. 6A to 7B are signal intensity plots showing d-CTC assays multiplexed for four different prostate cancer-specific transcripts per reaction. Both the theoretical model (FIGS. 6A and 7A) and cancer cell line data (FIGS. 6B and 7B) shown for two such reactions, Reactions 1 and 2, demonstrate that the theoretical model accurately predicts the experimental data.

FIGS. 8A to 13B are signal intensity plots showing d-CTC assays multiplexed for four different breast and lung cancer specific transcripts per reaction. Both the theoretical models (FIGS. 8A, 9A, 10A, 11A, 12A, and 13A) and cancer cell line data (FIGS. 8B, 9B, 10B, 11B, 12B, and 13B) shown for six such reactions, Reactions 1 through 6, each with different combinations of markers, demonstrate that the theoretic model accurately predicts the experimental data.

FIG. 14 is a bar graph showing droplet PCR signal for seven different biomarkers (PIP, PRAME, RND3, PKP3, FAT1, S100A2, and AGR2) from 1 ng of non-amplified cell-line cDNA and from 1μl of pre-amplified product after 10, 14, and 18 cycles of Specific Target Amplification (STA) pre-amplification, demonstrating the significant enhancement of droplet PCR signal from STA pre-amplification.

FIGS. 15A to 15C are graphs that show the results of CTC detection in patients using the new d-CTC assay methods for three different sets of patients with lung cancer (FIG. 15A), breast cancer (FIG. 15B), and prostate cancer (FIG. 15C). In each, the healthy patients had no CTCs.

FIG. 16 is a horizontal bar graph that shows the results of patient prostate cancer data using a multiplexed d-CTC assay method described herein testing for the nine biomarkers recited in the figure (AGR2, Dual, FAT1, FOLH1, HOXB13, KLK2, KLK3, STEAP2, and TMPRSS2). 91 percent of cancer patients had detectable CTCs (10 of 11 patients), 24 of 28 samples contained detectable CTCs (86%), and 0 of 12 (0 percent) of healthy donor (HD) blood samples contained CTCs.

FIG. 17 is a series of signal intensity plots showing d-CTC assays multiplexed for for two different reactions (Reaction 1 (TMPRSS2, FAT1, KLK2, and STEAP2), left column, and Reaction 2 (KLK3, HOXB13, AGR2, and FOLH1), right column) for blood samples from a metastatic prostate cancer patient (top row), a localized prostate cancer patient (middle row), and from a healthy donor control sample (bottom row). In each case there were no CTCs in the healthy donor (HD) samples, but clear evidence of CTCs in the cancer samples.

FIG. 18 is a multiple bar graph illustrating the relative proportion of androgen receptor signaling genes in CTCs measured over time to provide a readout of drug response in a prostate cancer patient treated with Abiraterone®.

FIGS. 19A and 19B are graphs showing non-amplification versus 18 cycles of SMARTer pre-amplication. FIG. 19A is a bar graph that shows the level of amplicon amplification efficiency for different target regions that is consistent among the three replicates (WTA1, WTA2, WTA3). FIG. 19B is a graph that shows that using 18 cycles of SMARTer pre-amplification provides an increase in signal of approximately four orders of magnitude (10⁸ vs 10⁴) compared to a non-pre-amplified sample.

FIGS. 20A to 20C are graphs that show the results of testing of 11 markers in a multiplexed liver cancer assay. FIGS. 20A to 20C show the total droplet numbers in 21 hepatocellular carcinoma (HCC) patients (FIG. 20A), 13 chronic liver disease (CLD) patients (FIG. 20B, no significant detectable droplets) and 15 healthy donors (HDs) (FIG. 20C, no significant detectable droplets).

FIGS. 21A and 21B are graphs that show the results of a 14 marker multiplexed lung cancer assay. FIG. 21A shows the assay results for the 8 metastatic lung cancer patients and 8 healthy donors (all negative). FIG. 21B shows that all of the droplet counts per ml of blood in the cancer patients (8 of 8) were higher than in all healthy donors giving a detection rate of 100% in this assay.

FIG. 22 is a graph that shows the results of a breast cancer assay for a multiplexed eleven marker assay used in a field of 9 metastatic breast cancer patient, 5 localized breast cancer patients, and 15 healthy donors. The results show that the assay detects cancer in 7 of 9 metastatic breast cancer patients, 2 of 5 localized breast cancer patients, and none of the healthy donor samples.

FIGS. 23A and 23B are graphs that show the results of ARv7 detection in metastatic breast cancer patients. FIG. 23A is a bar graphs that shows the results for samples from 10 metastatic breast cancer patients and 7 healthy donors processed though the CTC-Chip as described herein. FIG. 23B shows that five of the ten cancer patient samples were above the healthy donor background level giving a detection rate of 5 in 10, or 50%.

FIG. 24A is a bar graph showing the detection rate of individual markers (PMEL, MLANA, MAGEA6, PRAME, TFAP2C, and SOX10) and a combined marker cocktail (SUM) in 34 melanoma patients.

FIG. 24B is a dot plot distribution of droplet signals detected in 34 melanoma patients for 182 draw points as compared to 15 healthy donors demonstrating an overall detection sensitivity above healthy donor background signal of 81% (based on draw points) and a specificity of 100% (by draw points).

### DETAILED DESCRIPTION

The present disclosure relates to methods and systems to obtain information from rare cancer cells in blood samples. These methods and systems combine the power of isolation techniques such as ultrahigh-throughput microfluidic techniques, for example, negative depletion techniques, e.g., those using negative depletion of hematopoietic cells to isolate untagged CTCs in a blood sample, with analysis techniques, such as droplet-based digital polymerase chain reaction (PCR) assays focused on ribonucleic acid (RNA) markers of specific cancer lineages. This strategy can also be applied to other CTC isolation technologies that provide partially purification of cells (e.g., filtration, positive tumor cell selection), although the quality of the RNA and hence the sensitivity of the assay will be inferior to the microfluidic technologies. Similarly, other digital PCR technologies applied to RNA are capable of detecting lineage-specific primers, although the sensitivity of the droplet-based assay is likely to be the highest.

The new methods described herein can be used not only for early detection of cancers based on the presence of the CTCs in the blood, but also for tumor burden quantification as well as to monitor CTCs from a particular tumor over time, e.g., to determine any potential changes in specific tumor marker genes present in the CTCs as well changes in the tumor as the result of specific therapies, e.g., in the context of a Clinical trial or a particular therapy.

### General Concepts of the Assay Methods

The isolation techniques are used to enrich CTCs from a blood sample, e.g., using ultrahigh-throughput microfluidic such as the so-called “CTC-iChip” described in, for example, International PCT Application WO 2015/058206 and in Ozkumur et al., “Inertial Focusing for Tumor Antigen-Dependent and -Independent Sorting of Rare Circulating Tumor Cells,” Sci. Transl. Med., 5:179ra47 (2013). The CTC-iChip uses a CTC antigen-independent approach in which WBCs in the blood sample are labeled with magnetic beads, and the sample is then processed through two enrichment stages. The first stage uses deterministic lateral displacement to remove small and flexible cells/particles (RBCs, platelets, unbound magnetic beads, and plasma) while retaining larger cells (CTCs and WBCs). The second stage moves all cells into a narrow fluid stream using inertial focusing and then uses a magnetic field to pull bead-labeled WBCs out of the focused stream, leaving highly enriched CTCs. The CTC-iChip product from 10 ml of whole blood typically contains <500,000 RBCs, <5,000 WBCs, and a variable number of CTCs.

Some analysis techniques further enrich and analyze the isolated CTCs, e.g., as obtained from the CTC-iChip, e.g., using droplet microfluidics. Some basic information on droplet microfluidics is described generally in Jeremy et al., “Ultrahigh-Throughput Screening in Drop-Based Microfluidics for Directed Evolution,” Proc. Natl. Acad. Sci. USA, 107:4004 (2010).

As used herein, the droplet microfluidic techniques can, in certain implementations, include encapsulation of single cells, RT-PCR reagents, and lysis buffer into droplets of typically non-aqueous liquids (e.g., fluorocarbons, hydrofluorocarbons, mineral oil, silicone oil, and hydrocarbon oil; surfactants can also be include in the non-aqueous liquid, e.g., Span80, Monolein/oleic acid, Tween20/80, SDS, n-butanol, ABIL EM90, and phospholipids), in the size range of, e.g., about 0.5 pL to 15 nL in volume and, e.g., 10 to 300 μm, e.g., 20 to 100 μm, e.g., 30 to 50 μm, e.g., 35 μim in diameter. As used in the new methods described in the present disclosure, these techniques further include amplification of cancer-specific transcripts within the droplets to produce a fluorescent signal, and sorting of amplification-positive drops. This approach results in isolation of pure CTCs that can be sequenced and analyzed for the purposes of diagnosis and individualized drug therapy. Due to the high heterogeneity of CTCs, it is useful to use multiplexed amplification to detect as many CTCs as possible. Thus, instead of using one pair of primers in the PCR mixture, one can increase the probability of detecting and sorting CTCs using a combination of tumor specific primers. For additional information on the use of PCR for sorting cancer cells, see, e.g., Eastburn et al., “Identification and genetic analysis of cancer cells with PCR-activated cell sorting,” Nucleic Acids Research, 2014, Vol. 42, No. 16 e128.

In the new assay methods CTCs are lysed to release RNA molecules, which are representative of the genes expressed in a cancer cell. Most are “lineage” specific, rather than cancer specific, for example any prostate cell (whether cancerous or not) expresses these markers. However, normal blood cells do not, and the fact that the signal is derived from a cell circulating in the bloodstream defines it as an abnormal signal. By converting the RNA to a cDNA, we can now PCR amplify this lineage signal. We use droplet digital PCR, which is extraordinarily sensitive, allowing to convert the signal from a single cancer cell (i.e., one signal in an imaging assay) into thousands of positive immunofluorescent droplets. The combination of multiple, highly curated gene transcripts ensures high sensitivity and specificity for cancer, and also allows for functional insights (as in the status of hormone responsive pathways in prostate and breast cancers).

As noted, the new assay methods focus on the detection and analysis of high quality RNA rather than DNA. While there has been considerable work on DNA mutation detection in plasma and in CTCs, the present methods rely on RNA markers for the following reasons:
1. DNA mutations are not tumor specific, and the discovery that a healthy individual has some unidentified cancer cells in the blood is a very difficult clinical situation. In contrast, by selecting tumor-specific RNAs (e.g., prostate vs lung), the new methods can identify the source of cancer cells in the blood.
2. DNA mutations are very heterogeneous and besides a few recurrent mutations shared by many cancers, most blood-based mutation detection strategies require pre-existing knowledge of the mutations present in the primary tumor (i.e. not appropriate for screening for unknown cancers). In contrast, all tumor cells derived from specific organs express common lineage markers at the RNA level. Thus, a single cocktail of markers is used in the new methods for each individual type of cancer.
3. Low levels of CTCs are shed by invasive cancers before metastases are established (i.e., it is not too late for blood-based detection), but the presence of tumor cells in the blood connotes vascular invasion (i.e., invasive rather than indolent cancer). That is not the case for plasma DNA or plasma protein markers, which are leaked from dying cells in the primary tumor, and do not necessarily indicate vascular invasion. For example, serum PSA protein in the blood is shed by both benign prostate cells as well as primary prostate cancers. On the other hand, CTCs expressing PSA are shed only by invasive prostate cancers.
4. The analysis of RNA using the novel digital scoring technologies described herein is extraordinarily sensitive. However, free RNA is degraded in the bloodstream, and the use of isolation systems as described herein, such as microfluidic negative depletion systems (e.g., the CTC- Chip system) is unique in that the untagged tumor cells have high quality RNA which is extractable.

The choice of cDNA as a target molecule over DNA was made to not only to boost the signal originating from each tumor cell, but also to specifically target only tumor cell transcripts to the exclusion of white blood cell (WBC) transcripts. The boost in signal is a significant advantage, as it avoids the need for the isolation of CTCs to very high levels of purity. That is, it enables robust and repeatable results with products that contain one or more “isolated” CTCs that are still surrounded by hundreds or thousands of contaminating WBCs, e.g., leukocytes, in the same product. Nevertheless, the strategy of targeting cDNA made from RNA as used in the new methods allows the new assay methods to be exquisitely tailored for maximum specificity with minimal levels of CTC purity compared to prior approaches.

The CTC-iChip technology is highly efficient at isolating non-hematopoietic cells by microfluidic depletion of antibody tagged leukocytes. This feature of the CTC-iChip provides intact tumor-derived RNA (at levels far above those obtained using other technologies), and it is independent of tumor cell surface epitopes (which are highly heterogeneous among cancers and among epithelial vs mesenchymal cell subtypes within an individual cancer). Furthermore, even pre-apoptotic cancer cells whose antibody staining and selection is suboptimal for imaging analysis can provide a source of tumor-specific RNA that can be scored using the methods described herein. For all these reasons, an isolation technology or system that provides high quality RNA from intact CTCs with at least some reduction in the WBCs found in the sample along with the rare CTCs, such as a microfluidic negative depletion system, e.g., the CTC-iChip, is an important first step isolation before the tumor-specific digital readout is applied to the product.

The droplet-based digital detection of extremely rare molecules within a heterogeneous mixture was originally developed for PCR amplification of individual DNA molecules that are below detection levels when present within a heterogeneous mixture, but which are readily identified when sequestered within a lipid droplet before being subjected to PCR. The basic technology for droplet-based digital PCR (“Droplet Digital PCR (ddPCR)”) has been commercialized by RainDance and Bio-Rad, which provide equipment for lipid encapsulation of target molecules followed by PCR analysis. Important scientific advances that made this possible include work in the laboratory of David Weitz at Harvard and Bert Vogelstein at Johns Hopkins. For example, see U.S. Pat. Nos. 6,767,512; 7,074,367; 8,535, 889; 8,841,071; 9,074,242; and U.S. Published Application No. 2014/0303005. See also U.S. Pat. No. 9,068,181.

However, droplet digital PCR itself is not biologically significant unless coupled to a biological source of material, which is key to the new methods described herein. For instance detection of lineage-specific RNAs (the central focus of our detection strategy) does not distinguish between normal prostate epithelial cells and cancerous prostate cells. As such, detection of prostate-derived transcripts in the blood is not meaningful: they are present within debris from normal prostate cells or exosomes. It is only when coupled with the isolation of whole CTCs (i.e., intact CTCs in the blood) that the ddPCR assay achieves both extraordinary sensitivity and specificity. Hence these two technologies are ideally suited for each other, because the isolation systems provide high quality RNA, and the droplet-based digital PCR assays are focused on RNA markers in the new methods.

One additional aspect is important to the overall success of the new assay methods. As noted, the new assay methods described herein use cDNA made from total RNA, but key to this use is the identification of appropriate biomarkers that are tumor lineage-specific for each type cancer, yet are so unique as to be completely absent in normal blood cells (even with ddPCR sensitivity). The selection, testing, and validation of the multiple target RNA biomarkers for each type of cancer described herein enable the success of the new assay methods.

### Assay Method Steps

The new assay methods start with the isolation of partially pure CTCs using an isolation system, such as a microfluidic negative depletion system, up to and including the analysis of data from a droplet digital PCR instrument. There are eight main assay steps, some of which are optional, though generally provide better results:
1. isolating from the blood sample a product including CTCs and other cells present in blood; e.g. from a patient or a subject;
2. reducing a volume of the rare cell-containing product (optional);
3. isolating ribonucleic acid (RNA) molecules from the product, e.g., by cell lysis, and generating cDNA molecules in solution from the isolated RNA; e.g., by RT-PCR of RNA released from cells contained in the product;
4. cleanup of cDNA synthesized during the RT-PCR step (optional);
5. pre-amplifying the cDNA using gene-specific targeted preamplification probes, e.g., using the Fluidigm BioMark™ Nested PCR approach, or non-specific whole-transcriptome amplification, e.g., using the Clontech SMARTer™ approach (optional);
6. encapsulating cDNA molecules in individual droplets, e.g., along with PCR reagents;
7. amplifying cDNA molecules within each of the droplets in the presence of reporter groups configured to bind specifically to cDNA from CTCs and not to cDNA from other cells, e.g., using PCR;
8. detecting droplets that contain the reporter groups (e.g., “positive” droplets) as an indicator of the presence of cDNA molecules from CTCs in the droplets; and
9. analyzing CTCs in the detected droplets, e.g., to determine the presence of a particular disease in a patient or subject.

As described in further detail below, one of the important features of the new d-CTC assay methods is the careful selection of a number of target gene biomarkers (and corresponding primers) that deliver excellent sensitivity, while simultaneously maintaining nearly perfect specificity. A unique list of target gene biomarkers described herein (Table 1, below) was determined using bioinformatics analyses of publicly available datasets and proprietary RNAseq CTC data. Great care was taken to select markers that are not expressed in any subpopulations of leukocytes, but are expressed at a high enough frequency and intensity in CTCs to provide a reliable signal in a reasonably wide array of different and distinct patients. A specific set of markers was selected for each cancer type (e.g. prostate cancer, breast cancer, melanoma, lung cancer, pancreatic cancer, among others.)

The separate steps of the assay methods will now be described in more detail.

### 1. CTC Isolation

Patient blood is run through the CTC-iChip, e.g., version 1.3M or 1.4.5T and a sample is collected in a 15 mL conical tube on ice. CTC-iChips were designed and fabricated as previously described (Ozkumur et al., “Inertial Focusing for Tumor Antigen-Dependent and -Independent Sorting of Rare Circulating Tumor Cells,” Science Translational Medicine, 5(179):179ra47 (DOI: 10.1126/scitranslmed.3005616) (2013)).

The blood samples (˜20 mls per cancer patient) are collected in EDTA tubes using approved protocols. These samples are then incubated with biotinylated antibodies against CD45 (R&D Systems) and CD66b (AbD Serotec, biotinylated in house) and followed by incubation with Dynabeads® MyOne® Streptavidin T1 (Invitrogen) to achieve magnetic labeling of white blood cells (Ozkumur et al., 2013).

The sample is then processed through the CTC-iChip, which separates the blood components (red and white blood cells and platelets) as well as unconjugated beads away from the CTCs. The CTCs are collected in solution while the red blood cells, platelets, unconjugated beads and the tagged white blood cells are collected in a waste chamber. The process is automated and 10 ml of blood is processed in 1 hour.

### 2. Volume Reduction and Storage of the Rare Cell-Containing Product

To fully lyse all cells isolated in the product, it is preferable to reduce the product volume from a typical starting point of several milliliters to a final volume of about 100 μl. This can be achieved, for example, by centrifuging the product, and resuspending in pluronic buffer in preparation for cell lysis and generation of cDNA. At this point samples can be processed for long-term storage by adding RNAlater™ (ThermoFisher), followed by flash-freezing in liquid nitrogen and storage at −80 C.

### 3. Isolating RNA and Generation of cDNA from Cells in the Product

The RNA isolation step is important to the process to fully release all RNA molecules from cells in preparation for RT-PCR. A one-step, in-tube reaction can be used to minimize the risk of cell and RNA loss likely to be incurred during standard transfer steps. For example, one can use the lnvitrogen SuperScript III® First-Strand Synthesis Supermix® for qRT-PCR kit, by adding the RT-PCR mastermix directly to the pelleted product, pipetting to lyse fully, and performing the reaction according to the kit protocol targeting a 1:1 RNA:cDNA ratio. Once cDNA has been synthesized, RNase H is applied to the reaction to remove any remaining RNA. Alternatively, if one wants to perform whole transcriptome pre-amplification of the sample in a later step, cDNA can be synthesized using the SMARTer™ Ultra Low Input RNA Kit protocol, which uses proprietary oligonucleotides and reverse transcriptase enzyme.

### 4. Cleanup of cDNA Synthesized During RT-PCR

Another useful, yet optional, step in the process involves the removal of lysis reagents from the cDNA-containing solution. The presence of harsh detergents can lead to the destabilization of the droplets used in the ddPCR method, once the cDNA-containing solution is transferred to the ddPCR instrument. Detergent removal can be accomplished, e.g., through the use of Solid Phase Reversible Immobilization (SPRI). This technique uses coated magnetic beads to first bind cDNA of a specific size range, then allows removal of detergent-containing supernatant, and finally elution of pure cDNA for input into the ddPCR instrument. In addition to the cleanup of the RT-PCR, the SPRI process also accomplishes a size selection of cDNA, which reduces the number of non-target cDNA molecules that enter the ddPCR phase of the process, which in turn reduces background and noise.

### 5. Pre-Amplification

Pre-amplification of the cDNA is an optional step that increases the number of template molecules that can be detected in the droplet PCR step thus improving signal-to-noise ratio and boosting the confidence in a positive read-out. It can be a very powerful approach for the detection of markers that are expressed at low levels in CTCs, and for analyzing samples that contain very small numbers of possibly apoptotic CTCs, such as in the context of early detection of pre-metastatic disease. These two approaches have been modified to be applied in the workflow of d-CTC assay. Specific Targeted Amplification (STA), based on the Fluidigm BioMark™ Nested PCR protocol, relies on the use of primers specifically designed to amplify the region targeted by the probes used in the droplet PCR step (see Table 2). These primers were carefully designed and tested in conjuncture with their respective fluorescent probes to ensure efficient and specific amplification without increase in noise in healthy controls. Alternatively, whole transcriptome amplification, based on the SMARTer™ Ultra Low Input RNA Kit protocol, relies on the amplification of every transcript in the product, including both those found in WBCs and those found in CTCs, using random primers.

### 6. Encapsulation of cDNA Plus PCR Reagents in Droplets

Once cDNA has been synthesized and purified of contaminating detergents, the entire aggregate of cDNA molecules in solution plus qPCR reagents is divided into many tiny compartmentalized reactions, for example, by a droplet making instrument, e.g., a droplet generator such as the Biorad Automated Droplet Generator, which generates 20,000 droplets per sample. Each reaction consists of an extremely small droplet of non-aqueous fluid, e.g., oil (PCR stable, e.g., proprietary formulation from vendor), which contains Taqman-type PCR reagents with gene-specific primers and an oligonucleotide probe, and a small amount of sample. Once droplet generation is complete, the sample consists of an emulsion containing a vast number of individual PCR-ready reactions.

For this step, one can use the PCR probes and related primers for any one or two or more different target genes listed in Table 1 below for overall determination of tumor load, e.g., to determine tumor progression or response to therapy, in single or multiplex reactions. Thus, although in some cases a single set of PCR primers and probes for a particular gene from Table 1 can be included in each droplet, it is also possible to multiplex PCR primers and probes for two or more different genes in each droplet using different fluorescent probes for each primer/probe set, to maximize the detection of tumor cells, given the heterogeneity of gene expression in CTCs. It is also possible to multiplex PCR primers and probes for multiple genes targeting different cancer types in each droplet, thus enabling the broad yet specific detection of multiple tumor types in a single assay.

### 7. PCR of Droplet Encapsulated cDNA Molecules

Standard PCR cycling is performed on the entire emulsion sample using qPCR cycling conditions. The reaction is carried to 45 cycles to ensure that the vast majority of individual droplet-PCR volumes are brought to endpoint. This is important because, although the reaction is performed with Taqman-type qPCR reagents and cycled under qPCR conditions, the fluorescent intensity of the sample will not be measured during the PCR cycling, but rather in the next step.

### 8. Detection of Positive Droplets

Since each individual partitioned PCR is brought fully to endpoint before any measurement of fluorescence is performed, each individual droplet will be either a fully fluorescent droplet or will contain virtually no fluorescence at all. This enables the simple enumeration of all positive (fluorescent) and negative (non-fluorescent) droplets.

### 9. Analysis

Because the upstream RT-PCR targeted a 1:1 RNA:cDNA ratio, each positive droplet should represent a single originating RNA transcript. This interpretation depends on the number of individual droplets far exceeding the number of target cDNA molecules. In the new process, at one extreme we consider the possibility of a single CTC being isolated and lysed, releasing some number of RNA transcripts which are then reverse-transcribed 1:1 into cDNA, partitioned, PCR-amplified, and enumerated.

We estimate that in the case of a moderately expressed gene, such as the KLK3 gene in prostate cancer cells, each cell contains approximately 80-120 copies of KLK3 mRNA. The Biorad QX200 ddPCR System generates 20,000 droplets, which ensures that for small numbers of isolated CTCs and moderately-expressed target genes there will never be more than one target cDNA molecule per droplet. On the other hand, in cases where the numbers of CTCs reach dozens or hundreds, for moderately-expressing genes there will likely be multiple copies of target cDNA per droplet. In such cases, approximate numbers of originating transcript can be estimated using Poisson statistics.

### Novel Gene Panels to Enable Lineage-Specific Identification of CTCs

As discussed above, the identification of gene transcripts that are highly specific for cancer cells within the context of surrounding normal blood cells is central to the new methods. While many genes are known to be more highly expressed in cancer cells, the vast majority of these genes also typically have at least limited expression in normal tissues, including blood. Given the extraordinary sensitivity required for this assay, complete absence of signal in normal blood cells is essential for high confidence identification of tumor cells in the bloodstream.

Candidate tumor-specific transcripts used to detect CTCs in blood are first selected by analyzing publicly available gene expression data sets derived from breast, prostate, lung, pancreas, and liver cancers and melanoma, as well as our lab-generated single cell RNASeq data from CTCs isolated from breast, prostate and pancreatic cancer patients and mouse models of these cancers. Transcripts whose expression is restricted to tumors and absent or undetectable in blood components are chosen for further downstream analysis. Demonstrating and validating total absence of expression (with the highest level of sensitivity, i.e., Digital PCR assays) in normal blood cells is important. In general, we found that only ˜10% of candidate genes predicted based on computational models or RNA Seq data are truly negative in human blood samples.

In particular, candidate tumor-specific mRNA transcripts for the detection of CTCs were initially identified through the analysis of gene expression data sets (microarray and RNA-Seq) derived previously for human breast, prostate, lung, pancreas, hepatocellular, and melanoma cancers. Specific publically available data sets used for this analysis include The Cancer Genome Atlas (TCGA) (The Cancer Genome Atlas, available online at tcga-data.nci.nih.gov/tcga/tcgaHome2.jsp) and the Cancer Cell Line Encyclopedia (CCLE) (available online at broadinstitute.org/ccle/home; see also, Barretina et al., The Cancer Cell Line Encyclopedia enables predictive modelling of anticancer drug sensitivity, Nature 483:603-607 (2012)). In addition, single-cell RNA-seq gene expression data from CTCs isolated from human patients with breast, prostate, and pancreatic cancers were analyzed (GEO accession numbers GSE51827, GSE60407, and GSE67980) (Aceto et al., Circulating tumor cell clusters are oligoclonal precursors of breast cancer metastasis, Cell, 158:1110-1122 (2014); Ting et al., Single-Cell RNA Sequencing Identifies Extracellular Matrix Gene Expression by Pancreatic Circulating Tumor Cells, Cell Rep, 8:1905-1918 (2014); and Miyamoto et al., RNA-Seq of single prostate CTCs implicates noncanonical Wnt signaling in antiandrogen resistance, Science 349:1351-1356 (2015). Tumor specific transcripts identified through these databases were then compared to human leukocyte RNA-Seq gene expression data (GEO accession numbers GSE30811, GSE24759, GSE51808, GSE48060, GSE54514, and GSE67980). Transcripts that displayed significant differential expression, with high expression in tumors and low or undetectable expression in leukocytes, were then selected for further downstream analysis. Moreover, a literature search was performed to select additional candidate tumor-specific transcripts. Between 50 and 100 candidate genes were selected for each type of human cancer.

For each candidate gene within each specific cancer type, two to four sets of PCR primers were designed to span regions across the target transcript. Primers are synthesized by IDT (Integrated DNA Technologies), probes are labeled with FAM or HEX, ZEN, and IABkFQ to create a probe targeting the middle of the amplicon. Unique features of our PCR primer design methodology necessary for the successful application of digital PCR-based mRNA transcript detection in human CTCs include the following: 1) the specific targeting of the 3′ end of each mRNA transcript, given the proclivity of cellular mRNA transcripts to degrade from the 5′-end, particularly in unfixed, fragile cells such as CTCs; 2) the design of primers to generate amplicons that span introns in order to exclude the unintentional amplification of contaminating genomic DNA, for example from excess contaminating leukocytes in the enriched CTC mixture; and 3) the design of primers to inclusively amplify multiple splice variants of a given gene, given the uncertainty in some cases regarding the clinical relevance of specific splice variants.

The specificity of the primers was first tested by qRT-PCR using cDNA derived from cancer cell lines (representing breast, prostate, lung, pancreas, and liver cancers and melanoma). For each type of human cancer, 2 to 5 established cancer cell lines were cultured and used for initial testing to evaluate PCR primer performance and assess for expression of the target transcript in the specified cancer. To provide an initial test of specificity, the same primers were used to evaluate expression of the target transcript in leukocytes from healthy individuals who do not have a diagnosis of cancer. Leukocytes from a minimum of five different healthy individuals were tested in this phase of testing (mixture of male and female individuals—this was dependent on the type of cancer; i.e. candidate prostate cancer and breast cancer genes required the use of male or female healthy donors only, respectively).

Leukocytes from healthy individuals were isolated from whole blood using Cell Preparation Tubes with Sodium Heparin (CPT) (Becton, Dickinson, and Co., NJ) following product insert instructions. RNA extraction and first-strand cDNA synthesis was performed for cancer cell lines and isolated leukocytes using standard methods. The specificity of expression of each gene (using 2 to 4 distinct sets of primers for each gene) was tested using qRT-PCR (cell line cDNA as positive controls, leukocyte cDNA from healthy donors as negative controls, and water as an additional negative control). Transcripts present in cancer cell lines, but absent in leukocytes based on qRT-PCR testing were then selected for further validation by droplet digital PCR. The selection criteria to pass this stage of testing were highly stringent, and required qRT-PCR signal to be present in at least one cancer cell line and absent in all healthy donor leukocyte samples tested.

Target transcripts and specific primer pairs that passed the qRT-PCR stage of testing were further validated using droplet digital PCR. For this stage of testing, the CTC-iChip (see, e.g., Ozkumur et al., “Inertial focusing for tumor antigen-dependent and -independent sorting of rare circulating tumor cells,” Sci Transl Med, 5, 179ra147 (2013) was used to process whole blood samples donated by healthy individuals. The CTC-iChip performs negative depletion of red blood cells, platelets, and leukocytes from whole blood, and generates a sample product that is enriched for cells in the blood that do not express leukocyte markers, including CTCs (which should not be present in healthy individuals). For each blood sample, the product from the CTC-iChip was supplemented with an RNA stabilization solution (RNAlater®, Life Technologies) and processed for RNA extraction and cDNA synthesis using standard methods. Droplet digital PCR (Biorad, CA) was then used to quantitate the number of transcripts present in each sample based on the specific primer pairs being tested. Samples assessed by droplet digital PCR during this phase of testing included cDNA from cancer cell lines, leukocyte cDNA from healthy donors processed through the CTC-iChip (at least four healthy individuals per primer pair being tested), and water as a negative control.

Criteria for passing droplet digital PCR testing were stringent, and included: 1) the presence of transcript signal in cancer cell lines (at least one cell line with >10 positive droplets); 2) excellent signal-to-noise ratio represented by separation of signal between positive and negative (empty) droplets; 3) minimal or absent droplet signal in healthy donors (<3 droplets per healthy donor); and 4) absent droplet signal in water (0 positive droplets).

Primers that amplified transcripts specifically in cell lines and not in leukocytes in the above droplet digital PCR testing were then subjected to detailed testing of sensitivity of signal. Using single cell micromanipulation, precise numbers of cancer cells (1, 5, 10, 25, and 50 cells) were spiked into whole blood donated by healthy individuals, and then processed through the CTC-iChip. Each sample was then processed as above for testing with droplet digital PCR, and evaluated for sensitivity to ensure the signal was sufficient for the desired clinical application.

The above stringent procedure of evaluating candidate genes and primers using qRT-PCR and droplet digital PCR resulted in a final primer list consisting of approximately 10% of the initial list of 50-100 candidate genes for each type of cancer (total of approximately 400 initial candidate genes). These primers are then further evaluated for signal in patient CTCs using blood samples donated by cancer patients undergoing cancer treatment at the MGH Cancer Center, collected under an

IRB-approved clinical protocol. Key to this portion of the evaluation is a comparison with blood collected from healthy individuals without a diagnosis of cancer. The following Table 1 lists the primers and probes for that have been developed thus far using these methods for the specific detection of CTCs from patients with prostate, breast, hepatocellular, pancreatic, lung, and melanoma cancers using droplet digital PCR.

While a single gene for each cancer type could be used, the presence of multiple genes within each panel is useful both for sensitivity (CTCs are heterogeneous even within individual patients in their expression patterns) and specificity (detection of multiple gene signals confers added confidence that this represents a true cancer cell signature).

The gene list provided below in Table 1 includes transcripts that are unique to specific types of cancer (e.g., highly specific markers of prostate or breast or liver cancers), as well as genes that are shared by several cancer types, e.g., all epithelial cancer types (and thus may serve as pan-cancer markers), and genes that are induced in certain conditions (e.g., active androgen signaling in prostate cancer or active estrogen signaling in breast cancer). Thus, each type of cancer was assigned a specific panel of genes that is designed for optimal sensitivity, specificity, and clinically actionable information for the given cancer type.

In addition, primers described in Table 2 are designed to pre-amplify some of the genes listed in Table 1, while maintaining their high specificity. If STA is a method of choice, these nested primers become additional components of each cancer panel.

### Gene Lists for Different Types of Cancers

The following Table 1 provides a list of names of genes (with (Genbank ID) and Sequence Identification numbers (SEQ ID NO)), along with cancer types for which they are selective (Br: breast, Lu: lung, Li: liver, Pr: prostate, Panc: pancreatic, Mel: melanoma). In addition, optimized primer sets are listed for each gene (primers 1 and 2), along with the composition of the fluorescent primer probes (e.g., 6-FAM™ (blue fluorescent label) or HEX™ (green fluorescent label) for tagged probes, and ZEN-31ABkFQ quencher) for optimal visualization of the digital PCR product.

TABLE 1

| | Disease | Seq | | Seq | | Seq | |
|---|---|---|---|---|---|---|---|
| Gene | Group | ID | Primer 2 | ID | Primer 1 | ID | Probe |
| AGR2 | Br, Lu, | 1 | CTG ACA | 2 | CAA TTC | 3 | /56-FAM/ATG CTT ACG |
| (10551) | Li, Pr | | GTT AGA | | AGT CTT | | /ZEN/AAC CTG CAG ATA |
| | | | GCC GAT | | CAG CAA | | CAG CTC /3IABkFQ/ |
| | | | ATC AC | | CTT GAG | | |
| ALDH1 | Br, Lu, | 4 | GGT GGC | 5 | TGT CGC | 6 | /56-FAM/TTT TCA CTT |
| A3 (220) | Panc | | TTT AAA | | CAA GTT | | /ZEN/CTG TGT ATT CGG |
| | | | ATG TCA | | TGA TGG T | | CCA AAG C/31ABkFQ/ |
| | | | GGA A | | | | |
| CADPS2 | Br, Li, | 7 | CTC TGC | 8 | GCC TTG | 9 | /56-FAM/TCC GAC GTG |
| (93664) | Lu, Mel | | ATT TTT | | CAC TTC | | /ZEN/GTA CTG TCA TT |
| | | | GGA CAT | | CAT TAT | | ACC T/31ABkFQ/ |
| | | | AGG AG | | GAC | | |
| CDH11 | Br, Lu, | 10 | GAG GCC | 11 | GTG GTT | 12 | /56-FAM/CAT CCT CGC |
| (1009) | Panc | | TAC ATT | | CTT TCT | | /ZEN/CTG CAT CGT CAT |
| | | | CTG AAC | | TTT GCC | | TCT /3IABkFQ/ |
| | | | GC | | TTC TC | | |
| CDH3 | Br, Li, | 13 | GTT TCA | 14 | GCT CCT | 15 | /56-FAM/CTG CTG GTG |
| (1001) | Mel | | TCC TCC | | TGA TCT | | /ZEN/CTG CTT TTG TTG |
| | | | CTG TGC | | TCC GCT | | GT/3IABkFQ/ |
| | | | TG | | TC | | |
| COL8A | Br, Lu | 16 | GAT GCC | 17 | CCT CGT | 18 | /56-FAM/AGT ATC CAC |
| 1(1295) | | | CCA CTT | | AAA CTG | | /ZEN/ACC TAC CCC AAT |
| | | | GCA GTA | | GCT AAT | | ATA TGA AGG AAA |
| | | | | | GGT | | /31ABkFQ/ |
| EGFR | Br, Lu, | 19 | CTG CTG | 20 | TTC ACA | 21 | /56-FAM/CTG CCT GGT |
| (1956) | Li, Panc | | CCA CAA | | TCC ATC | | /ZEN/CTG CCG CAA AT |
| | | | CCA GT | | TGG TAC | | C/31ABkFQ/ |
| | | | | | GTG | | |
| FAT1 | Br, Lu, | 22 | GAT CCT | 23 | ATC AGC | 24 | /56-FAM/TCT TGT CAG |
| (2195) | Li, Mel, | | TAT GCC | | AGA GTC | | /ZEN/CAG CGT TCC CGG |
| | Pr, Panc | | ATC ACC | | AAT CAG | | /31ABkFQ/ |
| | | | GT | | TGA G | | |
| FAT2 | Br, Lu | 25 | CCT GGA | 26 | TCC TCC | 27 | /56-FAM/ACC TGC TAC |
| (2196) | | | TGC TGA | | ACT CAT | | /ZEN/ATC ACA GAG GGA |
| | | | CAT TTC | | CTC CAA | | GAC C/31ABkFQ/ |
| | | | TGA | | CT | | |
| FOLH1 | Pr | 28 | CAA TGT | 29 | TGT TCC | 30 | /56-FAM/ATG AAC AAC |
| (2346) | | | GAT AGG | | AAA GCT | | /ZEN/AGC TGC TCC ACT |
| | | | TAC TCT | | CCT CAC | | CTG A/31ABkFQ |
| | | | CAG AGG | | AA | | |
| HOXB13 | Br, Lu, | 31 | CAG CCA | 32 | CTG TAC | 33 | /56-FAM/CAG CAT TTG |
| (261729) | Pr | | GAT GTG | | GGA ATG | | /ZEN/CAG ACT CCA GCG |
| | | | TTG CCA | | CGT TTC | | G/31ABkFQ/ |
| | | | | | TTG | | |
| KLK2 | Pr | 34 | GCT GTG | 35 | GTC TTC | 36 | /56-FAM/TGG CTA TTC |
| (3817) | | | TAC AGT | | AGG CTC | | /ZEN/TTC TTT AGG CAA |
| | | | CAT GGA | | AAA CAG | | TGG GCA /31ABkFQ/ |
| | | | TGG | | GT | | |
| KLK3 | Pr | 37 | GTG TGC | 38 | GTG ATA | 39 | /56-FAM/AAA GCA CCT |
| (354) | | | TGG ACG | | CCT TGA | | /ZEN/GCT CGG GTG ATT |
| | | | CTG GA | | AGC ACA | | CT/3IABkFQ/ |
| | | | | | CCA TTAC | | |
| LSAMP | Mel | 40 | CAC ATT | 41 | GCG GAT | 42 | /56-FAM/TCC AAG AGC |
| (4045) | | | TGA GTG | | GTC AAA | | /ZEN/AAT GAA GCC ACC |
| | | | AAG CTT | | CAA GTC | | ACA /31ABkFQ/ |
| | | | GTC G | | AAG | | |
| MAGE | Mel | 43 | GAA GGA | 44 | GCT GAC | 45 | /56-FAM/TTG CCC TGA |
| A6-RM1 | | | GAA GAT | | TCC TCT | | /ZEN/CCA GAG TCA TCA |
| (4105) | | | CTG CCA | | GCT CAA G | | TGC /3IABkFQ/ |
| | | | GTG | | | | |
| MET | Br, Li, | 46 | CCA GTA | 47 | TGT CAG | 48 | /56-FAM/AGT CAT AGG |
| (4233) | Lu, Panc | | GCC TGA | | TGA TTC | | /ZEN/AAG AGG GCA TTT |
| | | | TTG | | TGT TCA | | TGG TTG T/31ABkFQ/ |
| | | | TGCAT | | AGG A | | |
| MLAN | Mel | 49 | ACT CTT | 50 | CCA TCA | 51 | /56-FAM/AAG ACT CCC |
| (2315) | | | ACA CCA | | AGG CTC | | /ZEN/AGG ATC ACT GTC |
| | | | CGG CTG | | TGT ATC | | AGG A/3IABkFQ/ |
| | | | A | | CAT | | |
| NPY1R | Br, Lu | 52 | GGA TCT | 53 | GAA TTC | 54 | /56-FAM/AGC AGG AGC |
| (4886) | | | GAG CAG | | TTC ATT | | /ZEN/GAA AAA GAC AAA |
| | | | GAG AAA | | CCC TTG | | TTC CAA AG/3IABkFQ/ |
| | | | TAC c | | AAC TGA | | |
| OCLN | Br, Lu, | 55 | AAG ATG | 56 | ACT CTT | 57 | /56-FAM/TGC AGA CAC |
| (100506658) | u | | GAC AGG | | TCC ACA | | /ZEN/ATT TTT AAC CCA |
| | | | TAT GAC | | TAG TCA | | CTC CTC G/3IABkFQ/ |
| | | | AAG TC | | GAT GG | | |
| PDZRN3 | Mel | 58 | TGT CCT | 59 | TGG ATC | 60 | /56-FAM/AGC TCC TCC |
| (23024) | | | GGC TGT | | CCT ATC | | /ZEN/CTG TCC ATC TCC |
| | | | TCA TTC | | TCT TGC | | T/3IABkFQ/ |
| | | | TG | | CA | | |
| PGR | Br | 61 | GGC AAT | 62 | GGA CTG | 63 | /56-FAM/ACA AGA TCA |
| (5241) | | | TGG TTT | | GAT AAA | | /ZEN/TGC AAG TTA TCA |
| | | | GAG GCA | | TGT ATT | | AGA AGT TTT GTA AGT |
| | | | A | | CAA GCA | | T/3IABkFQ/ |
| PKP3 | Br, Li, | 64 | CTG GTG | 65 | GGT CGC | 66 | /56-FAM/AGT GTC |
| (11187) | Lu, Panc | | GAG GAG | | TGG ATG | | CGC/ZEN/AGC AGC TCG |
| | | | AAC GG | | AAA GGT T | | AA/3IABkFQ/ |
| PMEL | Mel | 67 | CAG GCA | 68 | ACA CAA | 69 | /56-FAM/TTT GGC TGT |
| (6490) | | | TCG TCA | | TGG ATC | | /ZEN/GAT AGG TGC TTT |
| | | | GTT TCCT | | TGG TGC | | GCT G/31ABkFQ/ |
| | | | | | TAA | | |
| PPL | Br, Lu, | 70 | GAG GAG | 71 | AGG TTC | 72 | /56-FAM/AGG AAC TCC |
| (5493) | Li | | AGA ATC | | AGG TAC | | /ZEN/ATT GAG GCG CAC |
| | | | AAC AAA | | TCC TTC | | AT/31ABkFQ/ |
| | | | CTG c | | CAG | | |
| RXRG | Mel | 73 | ATA CTT | 74 | AGC CAT | | |
| (6258) | | | CTG CTT | | TGT ACT | 75 | /56-FAM/CTC T GGT |
| | | | GGT GTA | | CTT TAA | | /ZEN/GGA GAC TCT GCG |
| | | | GGC | | CCC A | | AGA/3IABkFQ/ |
| RND3 | Br, Lu, | 76 | CCG AGA | 77 | GCG GAC | 78 | /56-FAM/ACG GCC AGT |
| (390) | Li, Mel, | | ATT ACG | | ATT GTC | | /ZEN/TTT GAA ATC GAC |
| | Panc | | TTC CTA | | ATA GTA | | ACA C/3IABkFQ/ |
| | | | CAG TG | | AGG A | | |
| S100A2 | Br, Lu, | 79 | CTG CCT | 80 | CTT ACT | 81 | /56-FAM/ACC TGG TCT |
| (6273) | Li, Panc | | TGC TCT | | CAG CTT | | /ZEN/GCC ACA GAT CCA |
| | | | CCT TCC | | GAA CTT | | TG/31ABkFQ/ |
| | | | | | GTC G | | |
| SCGB2A1 | Br | 82 | ACT TCC | 83 | GTC TTT | 84 | /56-FAM/CCATGA AGC |
| (4246) | | | TTG ATC | | TCA ACC | | /ZEN/TGC TGA TGG TCC |
| | | | CCT GCC | | ATG TCC | | TCA/3IABkFQ/ |
| | | | A | | TCC A | | |
| SFRP1 | Mel | 85 | CAA TGC | 86 | CTT TTA | 87 | /56-FAM/TGT GAC AAC |
| (6422) | | | CAC CGA | | TTT TCA | | /ZEN/GAG TTG AAA TCT |
| | | | AGC CT | | TCC TCA | | GAG GCC /3IABkFQ/ |
| | | | | | GTG CAA | | |
| | | | | | AC | | |
| SOX10 | Mel | 88 | CTT GTC | 89 | CTT CAT | 90 | /56-FAM/TTG TGC AGG |
| (6663) | | | ACT TTC | | GGT GTG | | /ZEN/TGC GGG TAC |
| | | | GTT CAG | | GGC TCA | | TGG/3IABkFQ/ |
| | | | CAG | | | | |
| SCHLAP1/ | Pr | 91 | TCC TTG | 92 | AGA TAC | 93 | /56-FAM/CCA ATG ATG |
| SET4 | | | GAT GAC | | CAC CTC | | /ZEN/AGG AGC GGG ATG |
| (101669 | | | TCT CCC | | CCT GAA | | GAG /3IABkFQ/ |
| 767) | | | TAC | | GAA | | |
| SCHLAP1 | Pr | 94 | AGA GGT | 95 | CTC TGG | 96 | /56-FAM/ACA TGC CTT |
| SET S | | | TTA ATG | | TCT GTC | | /ZEN/TCA CCT TCT CCA |
| | | | GGC TCA | | GTC ATG | | CCA /31ABkFQ/ |
| | | | CAG | | TAA G | | |
| AMACR | Pr | 97 | CAC ACC | 97 | TCA CTT | 99 | /56-FAM/AGA AAC GGA |
| (23600) | | | ACC ATA | | GAG GCC | | /ZEN/GGT CCA GCC AAG |
| | | | CCT GGA | | AAG AGT | | TTC /3IABkFQ/ |
| | | | TAAT | | TC | | |
| AR | Pr | 100 | CTT TCT | 101 | CTT GTC | 102 | /56-FAM/AAG CAG GGA |
| Variant7/ | | | TCA GGG | | GTC TTC | | /ZEN/TGA CTC TGG GAG |
| SET1 | | | TCT GGT | | GGA AAT | | AAA /31ABkFQ/ |
| (367) | | | CAT T | | GTT ATG | | |
| AR | Pr | 103 | GAG GCA | 104 | TGT CCA | 105 | /56-FAM/TGA AGC AGG |
| Variant | | | AGT CAG | | TCT TGT | | /ZEN/GAT GAC TCT GGG |
| 7 SET 3 | | | CCT TTCT | | CGT CTT | | AGA /3IABkFQ/ |
| | | | | | CG | | |
| AR | Pr | 106 | GCT CAC | 107 | TGG GAG | 108 | /56-FAM/TGA TTG CGA |
| Variant | | | CAT GTG | | AGA GAC | | /ZEN/GAG AGC TGC ATC |
| 12 SET: | | | TGA CTT | | AGC TTG | | AGT /31ABkFQ/ |
| | | | GA | | TA | | |
| AR | Pr | 109 | GAA AGT | 110 | GCA GCC | 111 | /56-FAM/TGA TTG CGA |
| Variant | | | CCA CGC | | TTG CTC | | /ZEN/GAG AGC TGC ATC |
| 12 SET | | | TCA CCAT | | TCT AGC | | AGT /31ABkFQ/ |
| UGT2B | Pr | 112 | CTC TGC | 113 | TTT CCT | 114 | /56-FAM/TTG GCT GGT |
| 15 SET | | | ACA AAC | | CGC CCA | | /ZEN/TTA CAG TGA AGT |
| 1(7366) | | | TCT TCC | | TTC TTA cc | | CCT CC/3IABkFQ/ |
| | | | ATT TC | | | | |
| UGT2B | PR | 115 | GGA AGG | 116 | GTG AGC | 117 | /56-FAM/TGG CTA CAC |
| 15 SET | | | AGG GAA | | TAC TGG | | /ZEN/ATT TGA GAA GAA |
| 5 | | | CAG AAA | | CTG AAC | | TGG TGG A/31ABkFQ/ |
| | | | TCC | | TATT | | |
| AFP | Li | 118 | AGG AGA | 119 | TCT GCA | 120 | /56-FAM/AAT GCT GCA |
| SET 1 | | | TGT GCT | | TGA ATT | | /ZEN/AAC TGA CCA CGC |
| (174) | | | GGA TTG | | ATA CAT | | TG/31ABkFQ/ |
| | | | TC | | TGA CCAC | | |
| AFP | Li | 121 | ACT GCA | 122 | TCA | 123 | /56-FAM/TTG CCC AGT |
| SET 2 | | | GAG ATA | | CCATIT | | /ZEN/TTG TTC AAG AAG |
| | | | AGT TTA | | TGC TTA | | CCA /3IABkFQ/ |
| | | | GCT GAC | | CTT CCT TG | | |
| STEAP2 | Br, Lu, | 124 | CAT GTT | 125 | TCT CCA | 126 | /56-FAM/ACA TGG CTT |
| (261729) | Pr, Panc | | GCC TAC | | AAC TTC | | /ZEN/ATC AGC AGG TTC |
| | | | AGC CTC T | | TTC CTC | | ATG CA/31ABkFQ/ |
| | | | | | ATT cc | | |
| TEAD3 | Br, Lu, | 127 | GAA GAT | 128 | CTT CCG | 129 | /56-FAM/AGC GTG CAA |
| (7005) | u | | CAT CCT | | AGC TAG | | /ZEN/TCA ACT CAT TTC |
| | | | GTC AGA | | AAC CTG | | GGC /3 lABkFQ/ |
| | | | CGA G | | TAT G | | |
| TFAP2C | Br, Lu, | 130 | GAT CAG | 131 | GAC AAT | 132 | /56-FAM/ACA GGG GAG |
| (7022) | Mel | | ACA GTC | | CTT CCA | | /ZEN/GTT CAG AGG GTT |
| | | | ATT CGC | | GGG ACT | | CTT /3IABkFQ/ |
| | | | AAA G | | GAG | | |
| TMPRS | Pr | 133 | CCC AAC | 134 | TCA ATG | 135 | /56-FAM/ACC CGG AAA |
| S 2 | | | CCA GGC | | AGA AGC | | /ZEN/TCC AGC AGA GCT |
| (7113) | | | ATG ATG | | ACC TTG | | /31ABkFQ/ |
| | | | | | GC | | |
| GPC3 | Li | 136 | TGC TGG | 137 | GCT CAT | 138 | /56-FAM/TCC TTG CTG |
| (2719) | | | AAT GGA | | GGA GAT | | /ZEN/CCT TTT GGC TGT |
| | | | CAA GAA | | TGA ACT | | ATC T/31ABkFQ/ |
| | | | CTC | | GGT | | |
| ALB | u | 139 | CTT ACT | 140 | CCA ACT | 141 | /56-FAM/ACA TTT GCT |
| (219) | | | GGC GTT | | CTT GTA | | /ZEN/GCC CAC TTT TCC |
| | | | TTC TCA | | GAG GTC | | TAG GT/31ABkFQ/ |
| | | | TGC | | TCA AG | | |
| G6PC | Li | 142 | GGA CCA | 143 | GCA AGG | 144 | /56-FAM/ACA GCC CAG |
| SET 1 | | | GGG AAA | | TAG ATT | | /ZEN/AAT CCC AAC CAC |
| (2538) | | | GAT AAA | | CGT GAC | | AAA /31ABkFQ/ |
| | | | GCC | | AGA | | |
| G6PC | u | 145 | CAT TTT | 146 | GAT GCT | 147 | /56-FAM/CTG TCA CGA |
| SET 2 | | | GTG GTT | | GTG GAT | | /ZEN/ATC TAC CTT GCT |
| | | | GGG ATT | | GTG GCT | | GCT CA/31ABKFQ/ |
| | | | CTG G | | | | |
| PRAME | Mel | 148 | GCC TTG | 149 | CTC TGC | 150 | /56-FAM/CAA GCG TTG |
| (23532) | | | CAC TTC | | ATT TTT | | /ZEN/GAG GTC CTG AGG |
| | | | CAT TAT | | GGA CAT | | C/31ABkFQ/ |
| | | | GAC | | AGG AG | | |
| AHSG | Li | 151 | ATG TGG | 152 | AGC TTC | 153 | 56-FAM/CCA CAG AGG |
| (197) | | | AGT TTA | | TCA CTG | | /ZEN/CAG CCA AGT GTA |
| | | | CAG TGT | | AGT GTT | | ACC /31ABkFQ/ |
| | | | CTG G | | GC | | |
| GPR143 | Mel | 154 | ACG GCT | 155 | CCA CTA | 156 | /56-FAM/TTC GCC ACG |
| (4935) | | | CCC ATC | | TGT CAC | | /ZEN/AGA ACC AGC AGC |
| | | | CTC CT | | CAT GTA | | /3IABkFQ/ |
| | | | | | CCT G | | |
| PTPRZ1 | Mel | 157 | TGC TCT | 158 | GGC TGA | 159 | /56-FAM/AGG CCA GGA |
| (5803) | | | GAC AAC | | GGA TCA | | /ZEN/GTC TTT GCT |
| | | | CCT TAT | | CTT TGT | | GACATT/3IABkFQ/ |
| | | | GC | | AGA | | |
| MUCL1 | Br | 160 | CAT CAG | 161 | TGT CTG | 162 | /56-FAM/ACT CCC |
| (118430) | | | CAG GAC | | TGC TCC | | AAG/ZEN/AGT ACC AGG |
| | | | CAG TAG | | CTG ATCT | | ACT GCT /31ABkFQ/ |
| | | | C | | | | |
| PIP | Br | 163 | TCA TTT | 164 | CTT GCT | 165 | /SHEX/CCT GCT CCT |
| (5304) | | | GGA CGT | | CCA GCT | | /ZEN/GGT TCT CTG CCT |
| | | | ACT GAC | | CCT GTTC | | G/31ABkFQ/ |
| | | | TTG G | | | | |
| PGR | Br | 166 | GGT GTT | 167 | ACT GGG | 168 | /56-FAM/AGT GGG CAG |
| (5241) | | | TGG TCT | | TTT GAC | | /ZEN/ATG CTG TAT TIT |
| | | | AGG ATG | | TTC GTA | | GCA C/31ABkFQ/ |
| | | | GAG | | GC | | |
| TFAP2C | Br, Lu | 169 | GTG ACT | 170 | CCA TCT | 171 | /56-FAM/TTC GGC TTC |
| (7022) | | | CTC CTG | | CAT TTC | | /ZEN/ACA GAC ATA GGC |
| | | | ACA TCC | | GTC CTC | | AAA GT/3IABkFQ/ |
| | | | TTA G | | CAA | | |
| SCGB2 | Br | 172 | ACT CTG | 173 | TCT AGC | 174 | /56-FAM/TAG CCC TCT |
| Al | | | AAA AAC | | AAT CAA | | /ZEN/GAG CCA AAC GCC |
| (4246) | | | TTT GGA | | CAG ATG | | /3IABKFQ/ |
| | | | CTG ATG | | AGT TCT | | |
| FAT1 | Br, Lu, | 175 | AGC TCC | 176 | GTC TGC | 177 | /56-FAM/ATC CCA GTG |
| (2195) | Pr | | TTC CAG | | TCA TCA | | /ZEN/ATA CCC ATT GTC |
| | | | TCC GAAT | | ATC ACC | | ATC GC/31ABkFQ/ |
| | | | | | TCA | | |
| FAT2 | Br, Lu, | 178 | GGA CAG | 179 | TGT GGG | 180 | /56-FAM/TGG AGG TGA |
| (2196) | Pr | | AGA GAA | | AGA ATA | | /ZEN/CTG TGC TGG ACA |
| | | | CAA GGA | | TAG GTG | | ATG /31ABkFQ/ |
| | | | TGA AC | | GAT TG | | |
| RND3 | Br, Lu | 181 | GCT TTG | 182 | CTG TCC | 183 | /56-FAM/ACA GTG TCC |
| (390) | | | ACA TCA | | GCA GAT | | /ZEN/TCA AAA AGT GGA |
| | | | GTA GAC | | CAG ACT | | AAG GTG A/31ABkFQ/ |
| | | | CAG AG | | TG | | |
| SFTPB | Lu | 184 | CCT GGA | 185 | CAT TGC | 186 | /56-FAM/CCG ATG ACC |
| (6439) | | | AAA TGG | | CTA CAG | | /ZEN/TAT GCC AAG AGT |
| | | | CCT CCTT | | GAA GTC | | GTG AG/31ABkFQ/ |
| | | | | | TGG | | |
| SCGB3 | Lu | 187 | CCA GAG | 188 | TCC CAG | 189 | /56-FAM/AAG GCA GTA |
| A2 | | | GTA AAG | | ATA ACT | | /ZEN/GCA GAG TAA CTA |
| (117156) | | | GTG CCA | | GTC ATG | | CAA AGG /31ABkFQ/ |
| | | | AC | | AAG c | | |
| SERPIN | Br, Lu | 190 | CCT CAA | 191 | GGA AGC | 192 | /56-FAM/TAG CAG TCT |
| A3 (12) | | | ATA CAT | | CTT CAC | | /ZEN/CCC AGG TGG |
| | | | CAA GCA | | CAG CAA | | A/31ABkFQ/ |
| | | | CAG c | | | | |
| SFRP2 | Br, Lu | 193 | TTG CAG | 194 | GCC CGA | 195 | /56-FAM/TTT CCC CCA |
| (6423) | | | GCT TCA | | CAT GCT | | /ZEN/GGA CAA CGA CCT |
| | | | CAT ACC | | TGA GT | | TT/31ABkFQ/ |
| | | | TT | | | | |
| CRABP2 | Br, Lu | 196 | CTC TTG | 197 | CCC TTA | 198 | /S6-FAM/TTT CTT |
| (1382) | | | CAG CCA | | CCC CAG | | TGA/ZEN/CCT CTT CTC |
| | | | TTC CTC | | TCA CTT CT | | TCC TCC CCT /31ABkFQ/ |
| | | | TT | | | | |
| AQP4 | Lu | 199 | TGG ACA | 200 | GGT GCC | 201 | /56-FAM/CCG ATC CTT |
| (361) | | | GAA GAC | | AGC ATG | | /ZEN/TGG ACC TGC AGT |
| | | | ATA CTC | | AAT CCC | | TAT CA/31ABkFQ/ |
| | | | ATA AAG | | | | |
| | | | G | | | | |
| TMPRS | Br, Lu | 202 | ATC TTC | 203 | CAG TTC | 204 | /56-FAM/CTC ACT CCA |
| S4 | | | CCT CCA | | CCA CTC | | /ZEN/GCC ACC CCA |
| (56649) | | | TTC TGC | | ACT TTC | | CTC/3IABkFQ/ |
| | | | TTC | | TCA G | | |
| GREM1 | Lu | 205 | TTT TGC | 206 | GCC GCA | 207 | /56-FAM/CCT ACA CGG |
| (26585) | | | ACC AGT | | CTG ACA | | /ZEN/TGG GAG CCC |
| | | | CTC GCT T | | GTA TGA G | | TG/31ABkFQ/ |
| FOXF1 | Lu | 208 | CGA CTG | 209 | CTC TCC | 210 | /56-FAM/CTG CAC CAG |
| (2294) | | | CGA GTG | | ACG CAC | | /ZEN/AAC AGC CAC AAC |
| | | | ATA CCG | | TCC CT | | G/3IABkFQ/ |
| NKX2-1 | Lu | 211 | TGC CGC | 212 | CAG GAC | 213 | /56-FAM/CCC |
| (7080) | | | TCA TGT | | ACC ATG | | GCCATC/ZEN/TCC CGC |
| | | | TCA TGC | | AGG AAC | | TTCA/31ABkFQ/ |
| | | | | | AG | | |
| NKX2-1 | Lu | 214 | AAG ATG | 215 | CGA AGC | 216 | /56-FAM/ATG TCG ATG |
| (7080) | | | TCA GAC | | CCG ATG | | /ZEN/AGT CCA AAG CAC |
| | | | ACT GAG | | TGG TC | | ACG A/3IABkFQ/ |
| | | | AAC G | | | | |
| AFP | Li | 217 | AGGAGAT | 218 | TCTGCATG | 219 | /56-FAM/AAT GCT GCA |
| (174) | | | GTGCTGG | | AATTATAC | | /ZEN/AAC TGA CCA CGC |
| | | | ATTGTC | | ATTGAC | | TG/31ABkFQ/ |
| AHSG | Li | 220 | ATGTGGA | 221 | AGCTTCTC | 222 | /56-FAM/CCA CAG AGG |
| (197) | | | GTTTACA | | ACTGAGTG | | /ZEN/CAG CCA AGT GTA |
| | | | GTGTCTG | | TTGC | | ACC/31ABkFQ/ |
| | | | G | | | | |
| ALB | Li | 223 | GAG ATC | 224 | CAA CAG | 225 | /56-FAM/AGA TAT ACT |
| (213) | | | TGC TTG | | AGG TTT | | /ZEN/TGG CAA GGT CCG |
| | | | AAT GTG | | TTC ACA | | CCC /3IABkFQ/ |
| | | | CTG | | GCAT | | |
| ALB | Li | 226 | CAT GGT | 227 | GAC GAT | 228 | /56-FAM/ACT TGT TGC |
| (213) | | | AGG CTG | | AAG GAG | | /ZEN/TGC AAG TCA |
| | | | AGA TGC | | ACC TGC | | AGCTGC/3IABkFQ/ |
| | | | TTT | | TTT G | | |
| ALB | Li | 229 | GCG CAT | 230 | GCT ATG | 231 | /56-FAM/ACC TCT TGT |
| (213) | | | TCT GGA | | CCA AAG | | /ZEN/GGA AGA GCC TCA |
| | | | ATT TGT | | TGT TCG | | GAA /3IABkFQ/ |
| | | | ACT c | | ATG | | |
| APOH | Li | 232 | TGA TGG | 233 | CCT GAA | 234 | /56-FAM/CCA GTT TCC |
| (350) | | | ATA TTC | | TCT TTA | | /ZEN/CAG TTT GGT ACA |
| | | | TCT GGA | | CTC TCT | | TTC TAT TTC TTC |
| | | | TGG C | | CTC CTT G | | C/3IABkFQ/ |
| FABP1 | Li | 235 | GCA CTT | 236 | ACC AGT | 237 | /56-FAM/AAC CAC TGT |
| (2168) | | | CAA GTT | | TTA TTG | | /ZEN/CTT GAC TTT CTC |
| | | | CAC CAT | | TCA CCT | | CCC TG/3IABKFQ/ |
| | | | CAC | | TCC A | | |
| FGB | Li | 238 | ACA TCT | 239 | TGG GAG | 240 | /56-FAM/ACC CTC CTC |
| (2244) | | | ATT ATT | | CCT CTT | | /ZEN/ATT GTC GTT GAC |
| | | | GCT ACT | | CTC TCT TC | | ACC /3IABkFQ/ |
| | | | ATT GTG | | | | |
| | | | TGT T | | | | |
| FGG | Li | 241 | TTC ATT | 242 | ACC TTG | 243 | /56-FAM/TGC CAT TCC |
| (2266) | | | TGA TAA | | AAC ATG | | /ZEN/AGT CTT CCA GTT |
| | | | GCA CAC | | GCA TAG | | CCA C/31ABkFQ/ |
| | | | AGT CTG | | TCT G | | |
| GPC3 | Li | 244 | AATCAGC | 245 | TGCTTATC | 246 | /56-FAM/TTC CAG GCG |
| (2719) | | | TCCGCTT | | TCGTTGTC | | /ZEN/CAT CAT CCA CAT |
| | | | CCTTG | | CTTCG | | CC/3IABkFQ/ |
| RBP4 | Li | 247 | CAG AAG | 248 | TCT TTC | 249 | /56-FAM/AGG CTG ATC |
| (5950) | | | CGC AGA | | TGA TCT | | /ZEN/GTC CAC AAC GGT |
| | | | AGA TTG | | GCC ATC | | T/31ABkFQ/ |
| | | | TAA G | | GC | | |
| TF | Li | 250 | AGA AGC | 251 | CAC TGC | 252 | /56-FAM/CCA |
| (7018) | | | GAG TCC | | ACA CCA | | GACACA/ZEN/GCC CCA |
| | | | GAC TGT | | TCT CAC A | | GGA CG/3IABkFQ/ |

Note that PRAME is also named MAPE (Melanoma Antigen Preferentially Expressed In Tumors), OIP4 (Opa-Interacting Protein OIP4), and CT130 (Cancer/Testis Antigen 130).

The following Table 2 lists nested primers designed to specifically pre-amplify the regions targeted by primers listed in Table 1.

TABLE 2

| Primer | Seq | | Seq | |
|---|---|---|---|---|
| name | ID | Nested Forward | ID | Nested Reverse |
| FAT1 | 253 | CAG ATG GAG GAG GAA | 254 | GTA TAC TGC CTG GAG TTC |
| | | GAT TCT G | | TCT G |
| FAT2 | 255 | CTG GTT CAG GTC TCC | 256 | GCT GTG ACT CTG AGC AAG |
| | | ATT ACA G | | TA |
| AGR2 | 257 | TGT CCT CCT CAA TCT | 258 | GAC AGA AGG GCT TGG AGA |
| | | GGT TTA TG | | TTT |
| PKP3 | 259 | CGG TGG CGT TGT AGA | 260 | AGA AGA TCT CTG CCT CCG A |
| | | AGA T | | |
| RND3 | 261 | CAA GAT AGT TGT GGT | 262 | AGG GTC TCT GGT CTA CTG |
| | | GGG AGA c | | ATG |
| TFAP2C | 263 | TTTGGATTTACCGCTTGG | 264 | GACTCCAGTGTGGGAGAG |
| | | G | | |
| S100A2 | 265 | GGG CCC ACA TAT AAA | 266 | CTG CTG GTC ACT GTT CTC |
| | | TCC TCA C | | ATC |
| PRAME | 267 | CTTCGCGGTGTGGTGAA | 268 | GCTGTGTCTCCCGTCAAA |
| PIP | 269 | CTG GGA CAC ATT GCC | 270 | CCA CCA TGC ATT CTT TCA |
| | | TTC T | | ATT CT |
| PGR | 271 | AAA CCC AGT TTG AGG | 272 | CCC TGC CAA TAT CTT GGG |
| | | AGA TGA G | | TAA T |
| SCGB2A | 273 | ACA GCA ACT TCC TTG | 274 | GCG GCA TCA CTG TCT ATG |
| 1 | | ATC CC | | AA |
| MUCL1 | 275 | CCT TGC CTT CTC TTA | 276 | AGC AGT GGT TTC AGC ATC A |
| | | GGC TTT | | |
| PGR | 277 | CAG ATA ACT CTC ATT | 278 | CTC TAA TGT AGC TTG ACC |
| | | CAG TAT TCT TGG | | TCA TCT |
| TFAP2C | 279 | GAG AAG TTG GAC AAG | 280 | GCT GAG AAG TTC TGT GAA |
| | | ATT GGG | | TTC TTT A |
| SCGB2A | 281 | GTT TCC TCA ACC AGT | 282 | AGT TGT CTA GCA GTT TCC |
| 1 | | CAC ATA GA | | ACA TA |
| FAT1 | 283 | GGG AAA GCC TGT CTG | 284 | TCG TAG CCT CCA GGG TAA |
| | | AAG TG | | TAG |
| FAT2 | 285 | GTT ACA GGT CTC CTA | 286 | GCT CAG CCT CTC TGG AAG |
| | | TCT ACA GC | | |
| RND3 | 287 | CTC TCT TAC CCT GAT | 288 | GGC GTC TGC CTG TGA TT |
| | | TCG GAT G | | |
| SFTPB | 289 | CCT GAG TTC TGG TGC | 290 | GGG CAT GAG CAG CTT CAA |
| | | CAA AG | | |
| SCGB3A | 291 | CCA CTG GCT TGG TGG | 292 | TCA ACA GAA ATG CCC AGA |
| 2 | | ATT T | | GTT |
| SERPIN | 293 | CTT CTC CAG CTG GGC | 294 | TGC TGT GGC AGC AGA TG |
| A3 | | ATT | | |
| SFRP2 | 295 | CGG TCA TGT CCG CCT | 296 | GCG TTT CCA TTA TGT CGT |
| | | TC | | TGT C |
| CRABP2 | 297 | CCC TCC TTC TAG GAT | 298 | AAC CCG GAA TGG GTG AT |
| | | AGC G | | |
| AQP4 | 299 | AAACGGACTGATGTCAC | 300 | TGGACAGAAGACATACTCATA |
| | | TGG | | AAGG |
| TMPRSS | 301 | CCCACTGCTTCAGGAAA | 302 | GTCAGACATCTTCCCTCCATTC |
| 4 | | CATA | | |
| GREM1 | 303 | GCCGCACTGACAGTATG | 304 | CAGAAGGAGCAGGACTGAAA |
| | | A | | |
| FOXF1 | 305 | AGC GGC GCC TCT TAT | 306 | GCG TTG AAA GAG AAG ACA |
| | | ATC | | AAC T |
| NKX2-1 | 307 | CTA CTG CAA CGG CAA | 308 | GGG CCA TGT TCT TGC TCA |
| | | CCT | | |
| NKX2-1 | 309 | CAG ACT CGC TCG CTC | 310 | CCT CCA TGC CCA CTT TCT T |
| | | ATT T | | |
| PIP | 311 | CCCAAGTCAGTACGTCC | 312 | GCCTAATTCCCGAATAACATC |
| | | AAAT | | AA |
| AGR2 | 313 | GCT TTA AAG AAA GTG | 314 | CTG TAT CTG CAG GTT CGT |
| | | TTT GCT G | | AAG |
| SOX10 | 315 | AAG TTC CCC GTG TGC | 316 | CTC AGC CTC CTC GAT GAA |
| | | ATC | | |
| MAGEA | 317 | GTGAGGAGGCAAGGTTC | 318 | GGCTCCAGAGAGGGTAGTT |
| 6 | | TG | | |
| TFAP2C | 319 | TTTGGATTTACCGCTTGG | 320 | GACTCCAGTGTGGGAGAG |
| | | G | | |
| PRAME | 321 | CTTCGCGGTGTGGTGAA | 322 | GCTGTGTCTCCCGTCAAA |
| GPR143 | 323 | ATC CTG CTG TAT CAC | 324 | CTG ACA GGT TTC AAA GAA |
| | | ATC ATG | | CCT |
| PMEL | 325 | CCAGTGCCTTTGGTTGCT | 326 | CAAGAGCCAGATGGGCAAG |
| MLANA | 327 | TGCCAAGAGAAGATGCT | 328 | CATTGAGTGCCAACATGAAGA |
| | | CAC | | C |
| PTPRZ1 | 329 | AAG AAG CTG CCA ATA | 330 | TGT CCA GAG AGG TGG ATG |
| | | GGG AT | | |

### Multiplex Digital Analysis of Gene Transcripts from CTC-Chip Products

To improve the detection of tumor-specific mRNA from minimal amounts of RNA derived from CTCs, we established a multiplex assay capable of testing many different gene transcripts from a minute amount of CTC-Chip product. This combines the higher sensitivity/specificity of using multiple independent genes, with the fact that the amount of input template is limited (and hence should not be diluted into multiple reactions). Our assay includes 4 genes per reaction, with each gene being resolved uniquely in 2-dimensional space by selecting different ratios of fluorescent conjugated primers. Thus, in a single reaction, we can independently measure 4 gene transcripts without having to dilute the template. For different cancers, we have gone as far as up to 4 different reactions (i.e., up to 20 different gene transcripts), and with application of nested RT-PCR digital assays, there is no limit to the number of reactions that can be performed.

This multiplex strategy achieves the ideal balance between analyzing multiple transcripts (and hence ensuring against heterogeneous variation in cancer cell expression patterns), but not diluting the input mat erial by performing multiple independent PCR reactions. Depending on tumor types and the number of genes required for optimal signal, we have developed assays ranging from 2-4 multiplex reactions (each multiplex reaction testing for 4-genes). Thus, without undue dilution of input template, we can interrogate the product of a single CTC for expression of anywhere from 8 to 16 different genes. It is important to the assay to be able to add the signal from all of these genes (i.e. cumulative signal), while also having individual gene results (to optimize signal/noise at the individual gene level, and also gather information from specific signaling pathways that each gene interrogates — for example androgen signaling in prostate CTCs).

To display the results of the multiplex reaction in a single view (and hence differentiate amplification of each gene is isolation), we varied the concentrations of the two fluorescent probes (FAM (blue) and HEX (green)). By doing this, each individual gene amplification reaction has a unique combination of FAM/HEX signal that reflects the composition of the gene-specific primers, and hence identifies the gene-specific PCR product. In 2-dimensional space, we can illustrate the signal position of 4 different gene amplification products produced from a single multiplex reaction. As applied to digital PCR using droplets to encapsulate each PCR reaction, this method separates the targets into individual clusters by modifying the binary signal amplitude of positive droplets, which are displayed quantitatively. As predicted, this method allows both cumulative scoring of total signal for multiple genes (e.g., 16 markers in a total of 4 reactions), while also retaining the ability to quantify the signal from each individual gene target.

Specific results of testing are detailed in the examples below.

### Applications of the d-CTC Assay Methods

The early detection of epithelial cancers at a time when they can be surgically resected or irradiated provides the best chance of cure, and the administration of adjuvant chemotherapy in the setting of minimal cancer dissemination is far more effective in achieving cure than the treatment of established metastatic disease. However, current efforts at early cancer detection suffer from lack of specificity. For instance widespread screening of men for prostate cancer, using serum PSA measurements is effective in uncovering early cancers, but it also identifies a much larger number of non-malignant prostate conditions (e.g., benign hypertrophy of the gland) or even cancers that are indolent and never destined to become invasive. As such, broad PSA screening is not recommended by public health organizations, because the number of complications (including deaths) from over-diagnosis match or even outweigh the calculated benefit in early cancer detection.

For other cancers, such as breast cancer, mammography is considered effective, but even then a large number of breast biopsies are performed to diagnose each true malignancy. For lung cancer, the recently recommended low dose CT scanning of individuals with a heavy cigarette smoking history is also likely to detect hundreds of innocent radiographic abnormalities for each true malignancy.

It is in this context that the addition of a blood-based ultra-sensitive readout for the presence of cancer cell-derived signatures would provide the required specificity. The d-CTC assays described herein can be used for both initial screening and as a confirmation of earlier screenings at a later time. For example, in some cases the assays can be used as a second-line test to validate a highly sensitive, but nonspecific screening test (e.g., PSA in prostate cancer). In other settings for which a cancer is highly lethal, but no screening approach currently exists (e.g., pancreatic cancer), routine periodic blood screening using the assays described herein may become the norm to monitor a patient's status or condition over time.

The new d-CTC readouts are also highly relevant to the serial monitoring of patients, e.g., seemingly healthy patients with a family history and/or genetic markers of a specific type of cancer, or patients with advanced or metastatic cancer. Imaging of CTCs is expensive and relatively insensitive, in that intact cells that stain appropriately for all required markers produce a single signal. The use of the new d-CTC assays described herein, in which each CTC (no matter how intact or pre-apoptotic) can give rise to hundreds of molecular signals, dramatically enhances the ability to detect and monitor CTCs in patients with known cancer, and to quantitatively monitor and analyze their response to therapeutic interventions. Beyond scoring for cell numbers through molecular markers, specific interrogation of mutations or cancer-associated rearrangements (e.g., EML4-ALK in lung cancer) can be achieved with comparable sensitivity.

In addition to providing a digital (quantitative) measure of CTCs present within a blood sample, the new d-CTC assay also allows analysis of specific signaling pathways that are unique to the tumor cells in the blood. For instance, a subset of prostate lineage-specific genes are driven by androgen signaling (such as PSA), while another subset are repressed by androgen signaling (such as PSMA). By analyzing these genes together, we can ascertain the status of androgen signaling within CTCs. Similarly, in breast cancer, expression of estrogen-responsive genes (such as progesterone receptor) provides a measure of the status of the estrogen-responsive pathway within CTCs. These measurements are particularly important in that therapeutic interventions in both prostate and breast cancers are derived to target the androgen and estrogen receptors, respectively. Thus, defining the total number of CTC signal in the blood, simultaneously with information about the effectiveness of the therapeutic agent in targeting and shutting off the critical pathway is important for therapeutic monitoring.

As discussed in the examples below, the new methods described herein are illustrated in prostate cancer, where the anti-androgenic agent abiratorone (e.g.,) ZZTIGA®)is effective in suppressing cancer progression, particularly in tumors that are still dependent on the androgen pathway.

### EXAMPLES

The invention is further described in the following examples, which do not limit the scope of the invention described in the claims.

### Example 1—Preliminary Testing and Validation of the Digital CTC Assay

To test the feasibility of CTC-Chip-Droplet assay, we first selected several transcripts that are specifically expressed in prostate tumor cells, but are absent in contaminating leukocytes. These were the prostate lineage specific markers KLK3 (kallikrein-related peptidase; aka Prostate Specific Antigen, or PSA), FOLH1 (Folate Hydrolase; aka Prostate Specific Membrane Antigen, or PSMA) and AMACR (alpha-methylacyl-CoA racemase), as well as EpCAM (Epithelial Cell Adhesion Molecule). PCR conditions were optimized using intron-spanning primers and ZEN double-quenched FAM-labelled probes from Integrated DNA Technologies (Coralville, Iowa) following standard qPCR protocols. These conditions were first tested with encapsulated cDNA from admixtures of cancer cells and leukocytes in order to explore the dynamic range of the system. Next, using manual isolation techniques for individually selecting cells, 0, 3, 6, 12, 25, and 125 prostate cancer LNCaP cells were progressively spiked into individual 5 ml aliquots of HD blood, followed by CTC-iChip processing, RT-PCR and droplet encapsulation using the RainDrop system. We chose KLK3 as the target transcript for this experiment as it is predicted to be modestly abundant. Using an intensity threshold of 5,000, we found that as few as 3 cells worth of KLK3 transcript were readily detected at approximately 250 droplets.

Based on these preliminary data, we tested the CTC-Chip Droplet assay in patients with metastatic and localized prostate cancer versus healthy controls. Each sample was run through the iChip, then CTC-containing product was run through droplet RT-PCR using the four prostate markers mentioned above: KLK3, AMACR, FOLH1 and EpCAM. Patients with either local or metastatic prostate cancer produced significantly higher positive droplet counts as compared to HD controls.

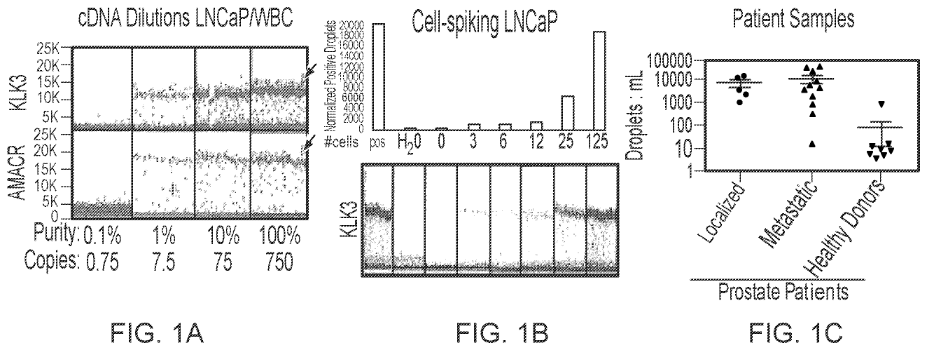

FIG. 1A shows cDNA dilutions prepared from total RNA of LNCaP prostate cancer cells, mixed with leukocytes and analyzed by droplet PCR using two different prostate primer sets. The results represent several purities and show good response of positive droplet number across this range.

FIG. 1B shows manually isolated LNCaP cells spiked into HD blood samples, run through the iChip, and subjected to droplet RT-PCR (KLK3 primer set). The results show excellent sensitivity down to low numbers of target cells.

FIG. 1C shows the analysis of blood samples from healthy controls, patients with localized (resectable) prostate cancer and metastatic prostate cancer, processed through the CTC-iChip, subjected to RT-PCR and droplet analysis using three prostate-specific and one epithelial-specific biomarkers (KLK3, AMACR, FOLH1, EpCAM). The results are shown for the total number of droplets/ml for all four markers combined.

These results suggest that the application of a droplet-based PCR readout to the CTC-iChip greatly enhances its sensitivity in detecting virtually all CTCs present in a biological specimen. Taken together, the CTC-iChip and Droplet-PCR represent two powerful microfluidic technologies that are highly compatible with each other and can be integrated in-line to create a new and highly sensitive and accurate biological assay.

### Example 2—Digital CTC Assay Protocol

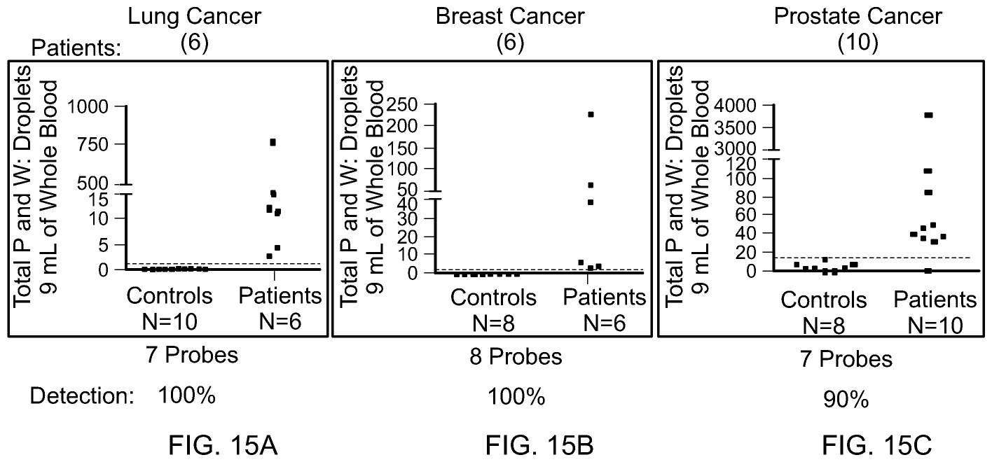

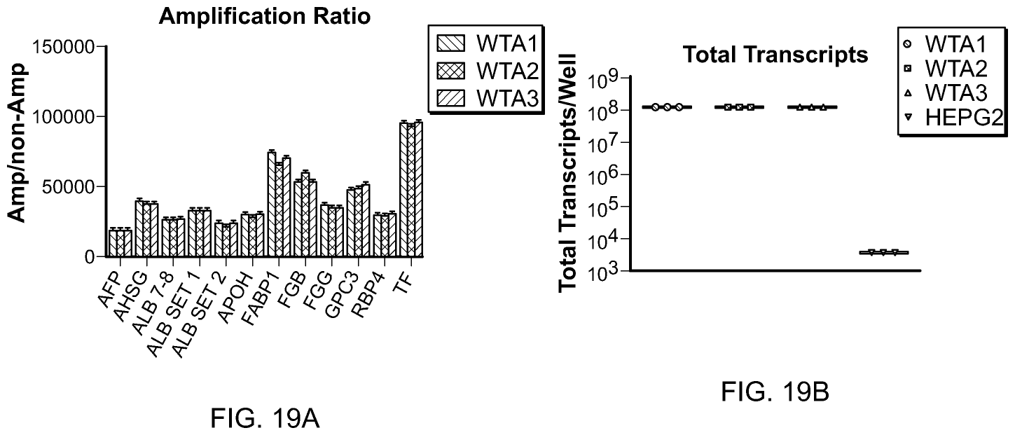

This example provides a general digital CTC assay protocol that can be used for the methods described herein. Different aspects of this general protocol were used in some of the Examples described herein. For example, Approach 1 of Step 3 of the protocol described below (relating to RNA purification to cDNA synthesis), was used to generate data for FIGS. 15A to 15C. Approach 2 in Step 3 was used to generate data for FIGS. 19A to 24B.
1. Patient blood is run through I-Chip, version 1.3M or 1.4.5T. Sample is collected in a 15 mL conical tube on ice.
2. Sample is spun down at 4C. Supernatant is decanted and SUPERase™
   In (DTT independent RNAse inhibitor)+RNALater® Stabilization Solution (prevents RNA degradation by inhibiting RNAses) is added to the pellet. Sample is flash frozen and placed at −80 until further processing. Samples are stable at −80.
3. There are two different processing protocols for RNA purification to cDNA synthesis that were used in the examples described below.

### Approach 1

a. Sample was thawed on ice.
b. Direct lysis of sample using detergents (NP40, Tween20).
c. Lysed sample was taken straight for cDNA synthesis (Superscript III).
d. After cDNA synthesis sample was purified via SPRI (Agencourt AMPure® XP beads) clean-up to clean up detergents and any nucleotides <100 bps.

### Approach 2

a. Sample was thawed on ice.
b. Sample was processed on RNeasy Qiagen Micro Kit. Protocol has some slight variations compared to traditional Qiagen recommendations.

Higher volumes of Buffer RLT (Lysis buffer) were used as well as higher ETOH concentrations. These modifications were made because of RNALater® addition to the sample.
c. After cDNA synthesis—sample was purified via SPRI (Agencourt AMPure XP beads) clean-up to clean up detergents and any nucleotides <100 bps.
4. cDNA (synthesized from Approach 1 or 2) can be processed in two different ways:
   a. cDNA was used directly for ddPCR; or
   b. cDNA was amplified used a Fluidigm BioMark™ Nested PCR approach (primers from genes used for nested PCR have been pre-validated). Amplified cDNA was diluted.
5. cDNA (from step 4a or 4b), Biorad Supermix™ for probes, primer or primers (for gene of interest; up to 4 different primers (FAM and HEX) can be multiplexed) were added in a total volume of 22 μl.
6. Droplets were generated (˜15,000-18,000 droplets per well).
7. Droplet Sample were put in a PCR machine. The PCR conditions were different than Biorad recommendations. We used a step-down rather than a slow ramp to ensure that all droplets reach the same temperature. This is different than what both RainDance and Biorad uses. Better results (i.e., more signal and more separation between positive and negative droplets) can be obtained with the step-down rather than the gradient.
8. After the PCR, positive droplets were counted in a ddPCR machine.
9. Data is collected and analyzed using TIBCO® Spotfire® analysis software.

The reagents, reagent concentrations, and reaction volumes are provided below:

### Reagents:

Biorad ddPCR™ Supermix for Probes (No dUTP)
IDT primers/probes (20x or 40x)
cDNA (1 ng/ul for cell lines)
Nuclease free water
Eppendorf semi-skirted 96 well plate (Only these plates work with the machine)

### Testing Relevant Cell Lines

Per single reaction:

| | |
|---|---|
| ddPCR Supermix | 11.0 μl |
| Primer (20x) | 1.10 μl |
| cDNA (1 ng/ul) | 1.10 μl |
| Water | 8.80 μl |
| TOTAL | 22.0 μl per well |

A master-mix containing ddPCR supermix, cDNA, and water were aliquoted into wells and 1.1 μl of each the primer was added to each well and mixed well.

Patient Samples

single reaction for Individual Genes

| | |
|---|---|
| ddPCR Supermix | 11.0 μl |
| Primer (20x) | 1.1 μl |
| cDNA (patient) | Up to 9.9 μl (Balance with water if less) |
| TOTAL | 22.0 μl per well |

Per single multiplexed reaction for Multiple Genes

| | |
|---|---|
| ddPCR Supermix | 11.0 μl |
| Primer 1 (40x) | .55 μl |
| Primer 2 (40x) | .55 μl |
| Primer 3 (40x) | .55 μl |
| Primer 4 (40x) | .55 μl |
| cDNA (patient) | 8.8 μl |
| TOTAL | 22.0 μl per well |

When testing multiple patients against a gene-specific primer or multiplexing primers against multiple genes, a master-mix, which includes the ddPCR supermix and primers, was aliquoted into wells followed by addition of patient cDNA to each well and mixed well.

### Example 3—Protocol for Gene Validation

The following protocol was used for selecting the specific marker genes listed in Table 1.
1. Transcripts that are unique to CTCs and not expressed in white blood ccells (WBCs), leukocytes, etc. were mined bioinformatically—Primary tumor and CTC gene expression data was compared to WBC gene expression datasets to isolate transcripts that were present only in primary tumor and/or CTCs.
2. Transcripts that passed a threshold cutoff were validated by qPCR.

3. Primers were synthesized by IDT. Probes were labeled with FAM/ZEN/IBFQ.
4. qPCR validation required that every transcript be validated by at least two independent primer sets on two different cell lines, 5 healthy donors WBCs (isolated via CPT column) and water as a negative control. 50 cycles for qPCR were used to confirm that expression of a transcript was only present in cell lines and not in healthy donors.
5. Transcripts that passed qPCR validation were validated on ddPCR with cell lines and healthy donors passed through the CTC-iChip (with and without cell spiking).
6. Panels of transcripts were multiplexed (up to 4 different genes per reaction) depending on disease of interest.

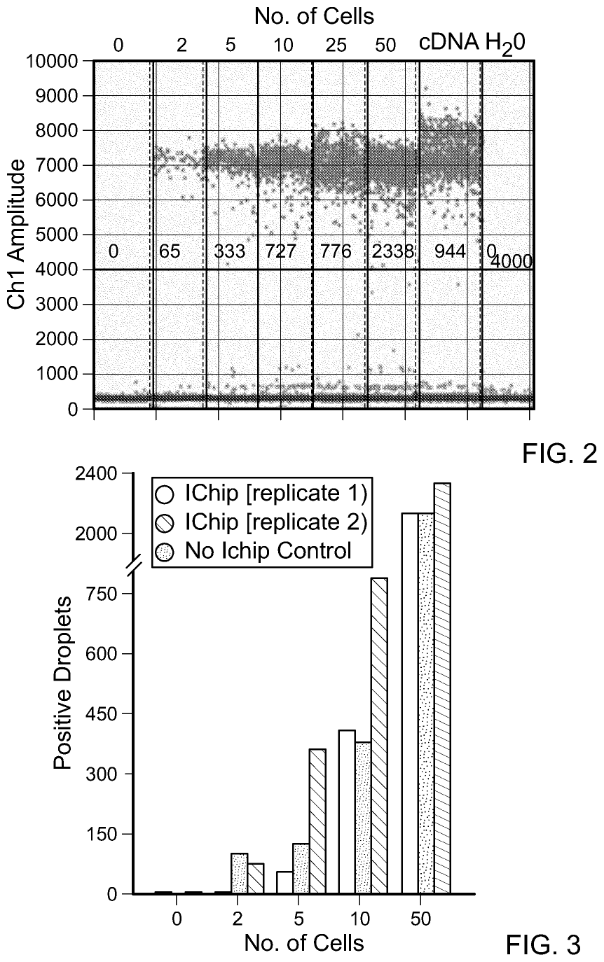

The validity of this strategy is shown below in a spiked cell experiment, in which a carefully measured number of tumor cells (from the LNCAP prostate cancer cell line) are individually micro-manipulated, added to control blood specimens, passed through the CTC-iChip and then analyzed by d-CTC assay as above. Increasing numbers of spiked cells show increasing numbers of digital signal as shown in FIG. 2, which illustrates the power of this protocol. FIG. 2 demonstrates the use of a single gene transcript (KLK3, also known as PSA, for prostate cancer) as a probe (in the assay, we use from 8-24 gene transcripts, thereby further increasing sensitivity). Here, we spike a calculated number of cancer cells (each cell is micro-manipulated, picked and introduced into 10 ml of control blood specimen). The blood is then processed through the CTC-Chip and subjected to digital readout as described above. No signal is observed in blood that has not been spiked with a single cancer cell. Introduction of 2 cells/10 ml of blood generates clear signal (65 positive droplets). In this case, the 10 CTC product was divided into 4 and run in quadruplicate, so the 64 droplets actually represent the digital signal derived from ¼ of a tumor cell.

This assay is both highly sensitive and reproducible. As shown in FIG. 3, the digital signal in these spiked cell experiments shows high reproducibility (2 independent replicates shown here), and the same amount of signal is seen when cells are spiked into buffer (rather than blood) and directly analyzed (without CTC-Chip processing). Thus, there is virtually no loss of signal when a tumor cell is diluted into billions of normal blood cells and then “re-isolated” using the CTC-Chip prior to digital readout.

### Example 4—Multiplex Digital Analysis of Gene Transcripts from CTC-Chip Product

We established a multiplex assay capable of testing many different gene transcripts from a minute amount of CTC-Chip product. This combined the higher sensitivity and specificity of using multiple independent genes, with the fact that the amount of input template is limited (and hence should not be diluted into multiple reactions). The new assays include multiple genes, e.g., 2, 3, 4, 6, 8, 10, or more genes per reaction, with each gene being resolved uniquely in 2-dimensional space by selecting different ratios of fluorescent conjugated primers. Thus, in a single reaction, one can independently measure 2, 3, 4, or more gene transcripts without having to dilute the template. For different cancers, one can run and analyze multiple different reactions (e.g., up to 20 different gene transcripts in four runs), and with application of nested RT-PCR digital assays, there is no limit to the number of reactions that can be performed.

To display the results of the multiplex reaction in a single view (and hence differentiate amplification of each gene is isolation), we varied the concentrations of the two fluorescent probes (FAM and HEX). By doing this, each individual gene amplification reaction has a unique combination of FAM/HEX signal that reflects the composition of the gene-specific primers, and hence identifies the gene-specific PCR product. In 2-dimensional space, we can illustrate the signal position of 4 different gene amplification products produced from a single multiplex reaction. As applied to digital PCR using droplets to encapsulate each PCR reaction, this method separates the targets into individual clusters by modifying the binary signal amplitude of positive droplets, which are displayed quantitatively. As predicted, this method allows both cumulative scoring of total signal for multiple genes (e.g., 16 markers in a total of 4 reactions), while also retaining the ability to quantify the signal from each individual gene target.
Probe 1: 100% FAM
Probe 2: 100% HEX
Probe 3: Mixture of FAM and HEX—sum up to 100%
Probe 4: Mixture of FAM and HEX—sum up to 100%

As shown in Tables 3 to 7, the following probe mixtures were used in the multiplex reactions:

TABLE 3
Multiplexing primers against 4 genes per
reaction (Melanoma)

| | | | | |
|---|---|---|---|---|
| FAM | HEX | Primer | FAM int | HEX Int |

Reaction 1

| | | | | |
|---|---|---|---|---|
| 100% | 0 | Sox10 | 6000 | 0 |
| 70% | 30% | SFRP1 | 4000 | 2500 |
| 30% | 70% | RND3 | 4500 | 5500 |
| 0% | 100% | TFAP2C | 0 | 6000 |

Reaction 2

| | | | | |
|---|---|---|---|---|
| 100% | 0 | PRAME | 11000 | 0 |
| 70% | 30% | MLANA | 8000 | 4000 |
| 30% | 70% | MAGEA6 | 5000 | 6000 |
| 0% | 100% | PMEL | 0 | 5500 |

Reaction 3

| | | | | |
|---|---|---|---|---|
| 100% | 0 | PMEL | 7000 | 0 |
| 70% | 30% | MLANA | 6000 | 3000 |
| 30% | 70% | MAGEA6 | 4000 | 5000 |
| 0% | 100% | MET | 0 | 4500 |

TABLE 4
Multiplexing primers against 4 genes per reaction
(Pan-Cancer/lineage)

| | | | Exp. FAM | Exp. HEX |
|---|---|---|---|---|
| FAM | HEX | Primer | Int | Int |
| 100 | 0 | TFAP2C | 9000 | 0 |
| 60 | 40 | PGR | 5100 | 1800 |
| 35 | 65 | SCGB2A1 | 2205 | 7800 |
| 0 | 100 | CADPS2 | 0 | 5000 |

TABLE 5
Multiplexing primers against multiple genes per reaction
(AR status in Prostate)
Multiplexing primers against 4 genes per reaction
(Prostate)

| | | | | |
|---|---|---|---|---|
| | | | Exp. FAM | Exp. HEX |
| FAM | HEX | Primer | Int | Int |

Reaction 1

| | | | | |
|---|---|---|---|---|
| 100 | 0 | TMPR2 | 5500 | 0 |
| 65 | 35 | FAT1 | 5525 | 1837.5 |
| 40 | 60 | KLK2 | 2440 | 2580 |
| 0 | 100 | STEAP2 | 0 | 4300 |

Reaction 2

| | | | | |
|---|---|---|---|---|
| 100 | 0 | KLK3 | 6600 | 0 |
| 70 | 30 | HOXB13 | 4340 | 1320 |
| 50 | 50 | AGR2 | 4050 | 3050 |
| 0 | 100 | FOLH1 | 0 | 5200 |

TABLE 6
Multiplexing primers against 4 genes per reaction
Epithelial-Mesenchymal Transition (EMT)

| | | | | |
|---|---|---|---|---|
| | | | Exp. FAM | Exp. HEX |
| FAM | HEX | Primer | Int | Int |

Reaction 1

| | | | | |
|---|---|---|---|---|
| 100 | 0 | PKP3 | 8000 | 0 |
| 75 | 25 | OCLN | 6000 | 1625 |
| 40 | 60 | CDH11 | 4000 | 3600 |
| 0 | 100 | S100A2 | 0 | 5000 |

Reaction 2

| | | | | |
|---|---|---|---|---|
| 100 | 0 | FAT1 | 8000 | 0 |
| 65 | 35 | FAT2 | 5200 | 1750 |
| 40 | 60 | COL8A1 | 3200 | 3900 |
| 0 | 100 | CDH3 | 0 | 6000 |

TABLE 7
Multiplexing primers against multiple genes per reaction

| | | Avg | Avg | |
|---|---|---|---|---|
| | Gene- | intensity | intensity | AR |
| Reaction | primer set | (FAM) | (HEX) | status |
| 1 | TMPRSS2 | 5500 | | ON |
| 1 | FAT1 | 8500 | 5250 | ? |
| 1 | KLK2 | 6100 | 4300 | ON |
| 1 | STEAP2 | 3350 | 4300 | ON |
| 2 | KLK3 | 6600 | | ON |
| 2 | FOLH1 | 6200 | 5200 | OFF |
| 2 | AGR2 | 8100 | 6100 | OFF |
| 2 | HOXB13 | 6500 | 4400 | OFF |

### Validation and Testing

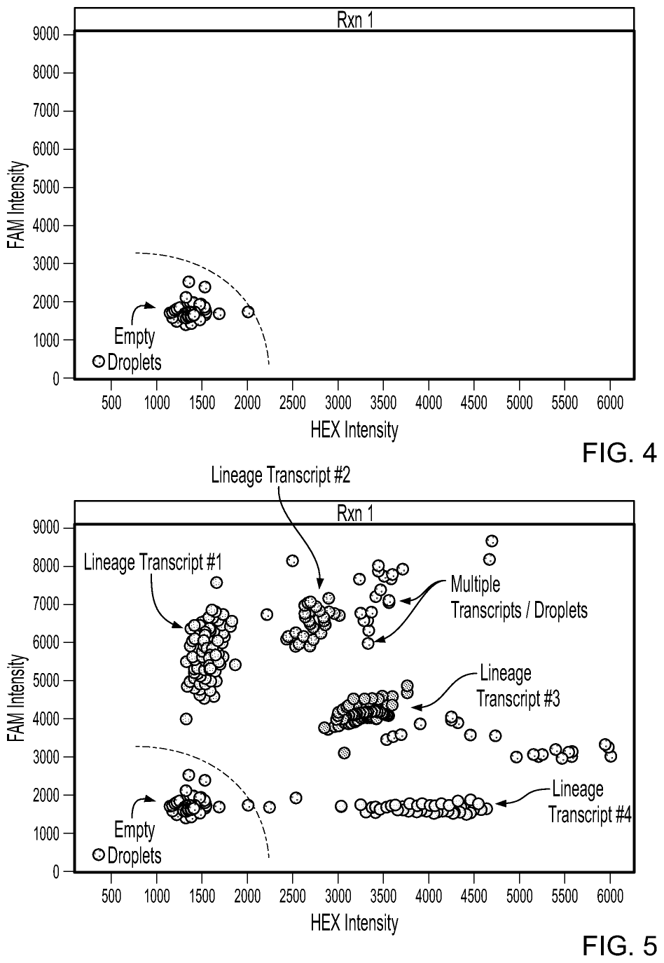

To validate and demonstrate the effectiveness of this multiplex strategy, we illustrated both the concept (using spiked cell experiments) and patient-derived samples. FIG. 4 shows the results of processing a normal control blood sample from a healthy donor (HD) through the CTC-Chip and subjected to d-CTC assay for 4 different gene transcripts, all of which are negative (i.e., blank droplets).

On the other hand, FIG. 5 is a representation of data from spiked cell experiments, prostate cancer cell lines introduced into blood and processed through the CTC-Chip, followed by digital assay, showed positive signal (fluorescent droplets) for each of the 4 lineage transcripts. These appeared at separate locations within the 2-Dimensional plot, based on differential fluorescence of two probes (color coded in picture). As the sample is overloaded with tumor cells, some droplets contained signal from more than one gene transcript (multiple genes per droplet are shown in gray).

The strategy of representing four different genes within each reaction was applicable to multiple different cancers, with specific lineage markers substituted for each tumor type. For instance, in prostate cancer, we predicted (theoretical model) a multiplex reaction with four quadrants (one gene per quadrant) for each of 2 reactions (total of 8 gene markers). The spiked cell experiment (prostate cancer cells introduced into control blood and processed through the CTC-iChip) precisely recapitulated the predicted results.

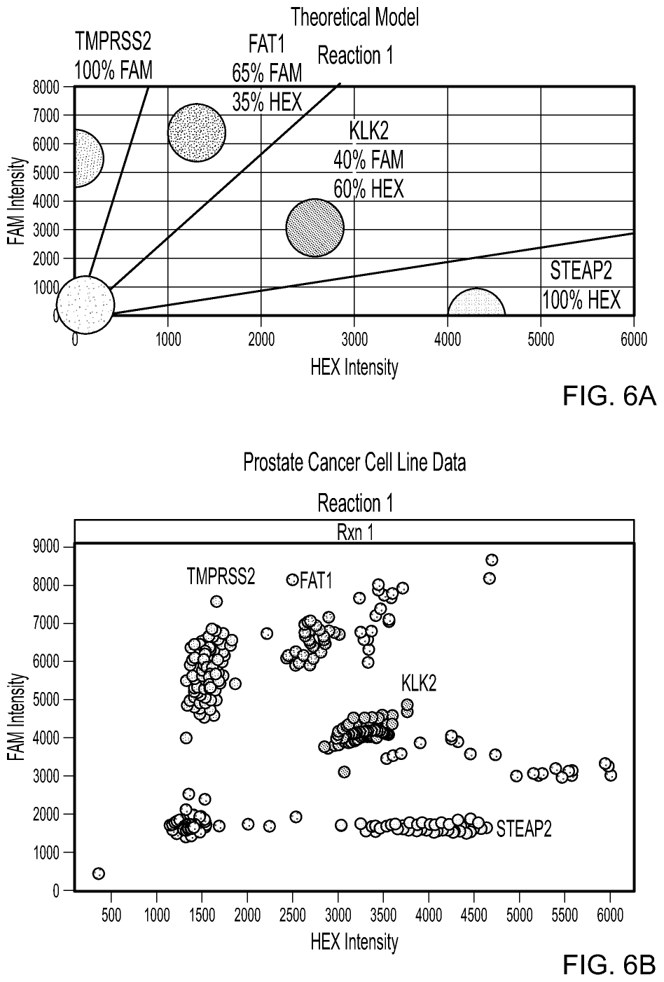

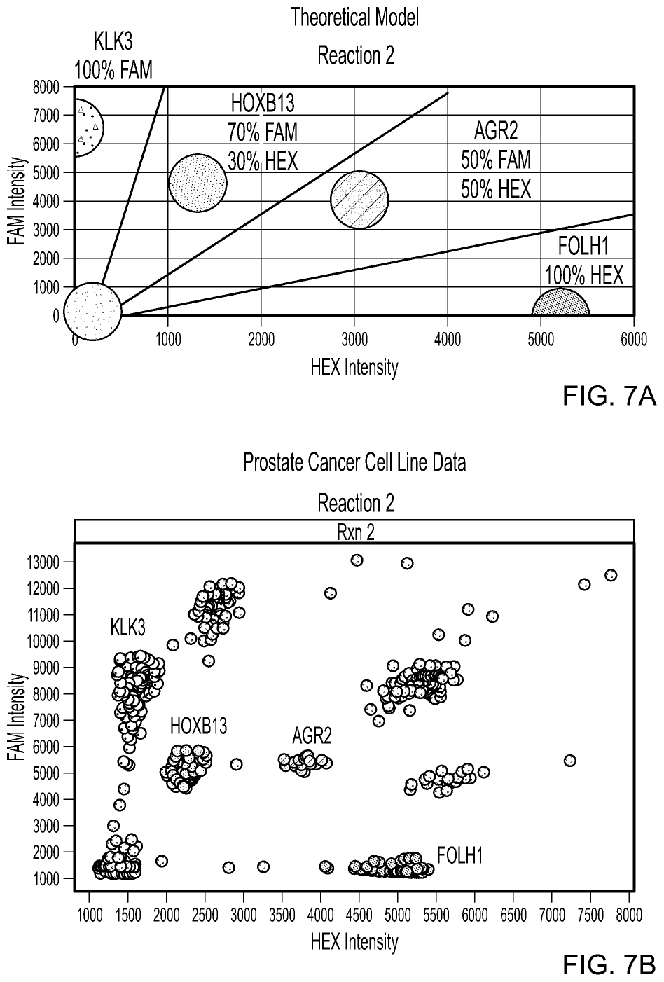

Furthermore, FIGS. 6A-6B and FIGS. 7A-7B show that when assembled together, our analytic program integrated all positive signals within quadrants, just as predicted from modeling, and allowing us to develop methods to score the specific gene signals. Multi-dimensional space analysis of signal allowed for automated analysis and scoring with high level accuracy. FIGS. 6A and 6B show the theoretical model and actual results, respectively, for a prostate cancer cell line for Reaction 1, and FIGS. 7A and 7B show the theoretical model and actual results, respectively, for the same prostate cancer cell line for Reaction 2.

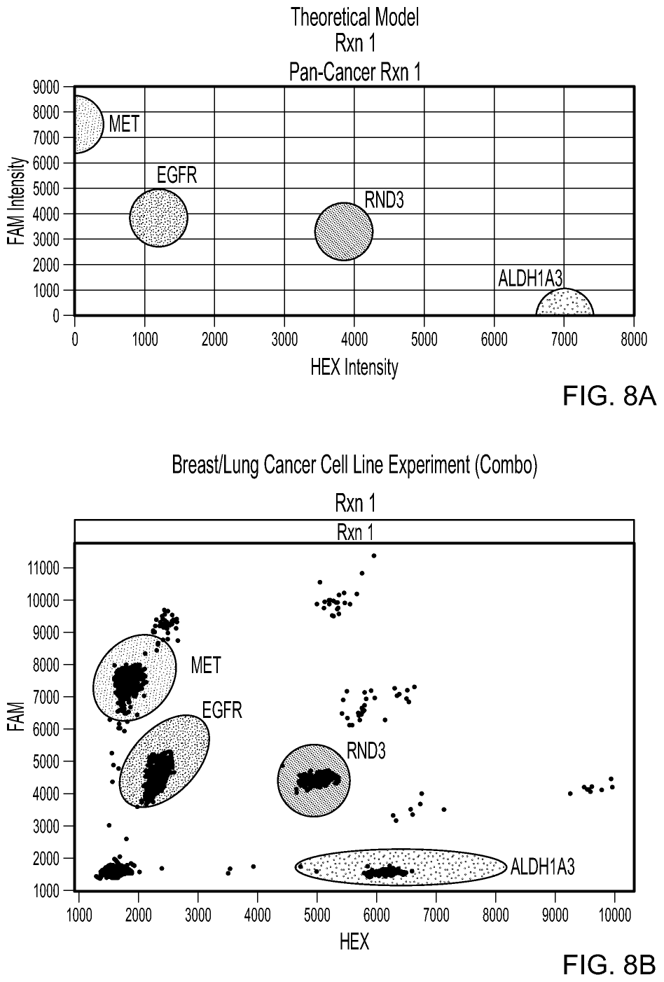

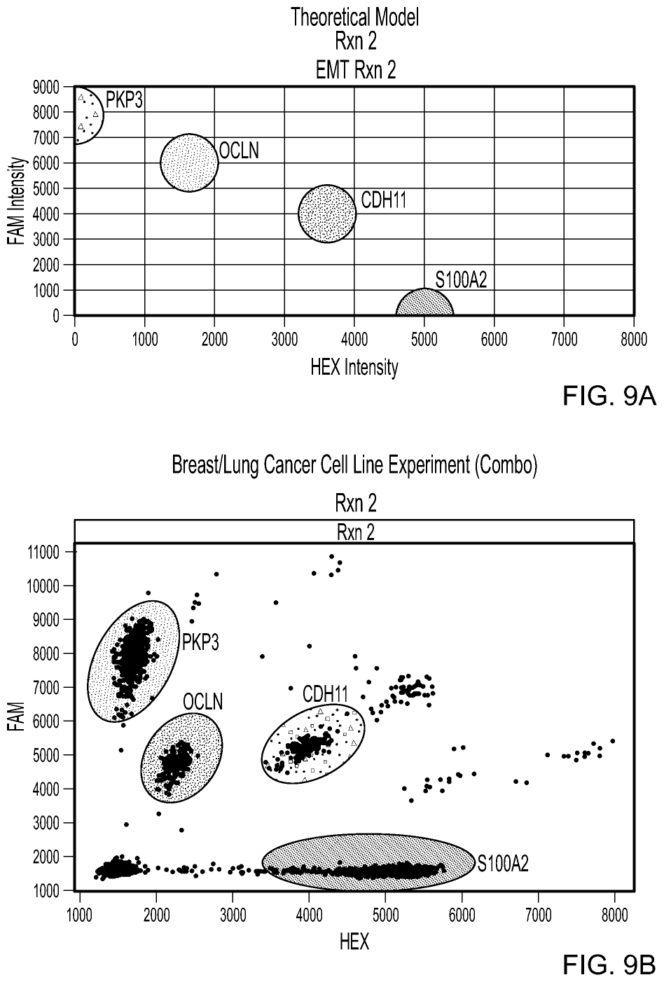

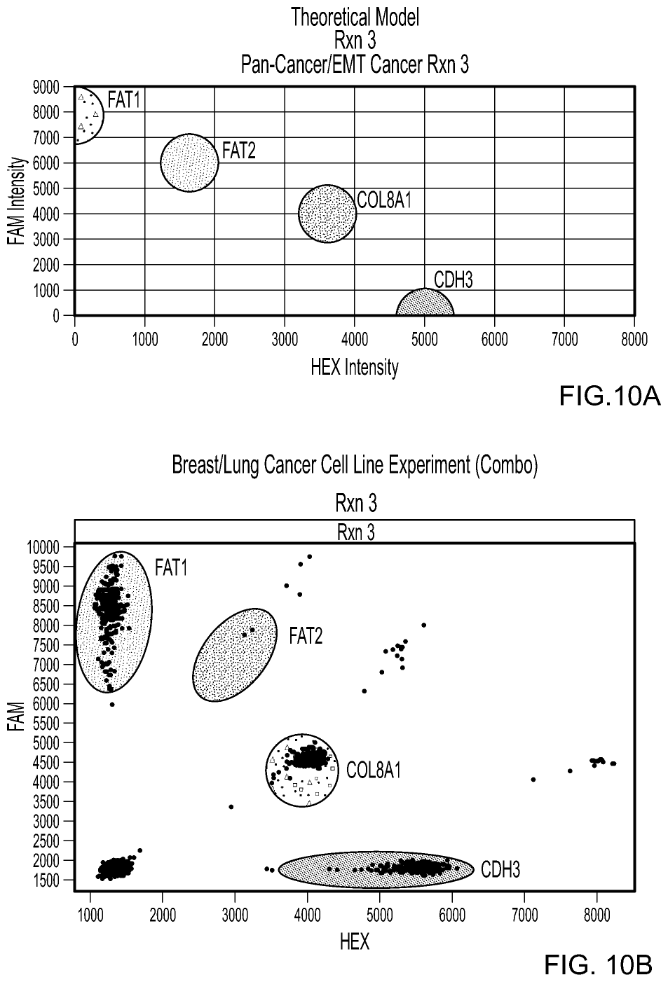

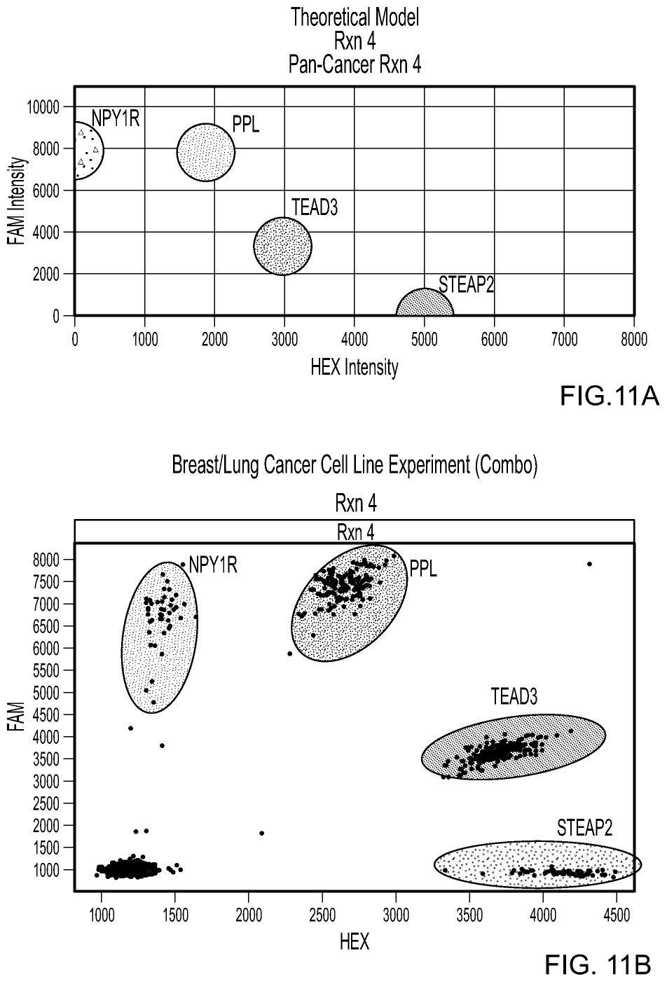

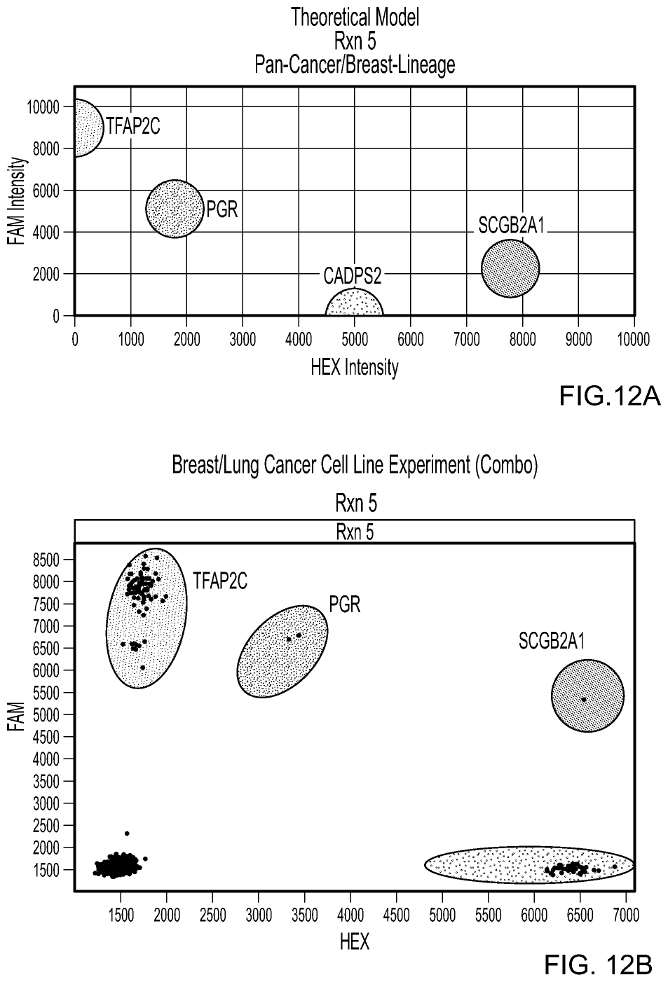

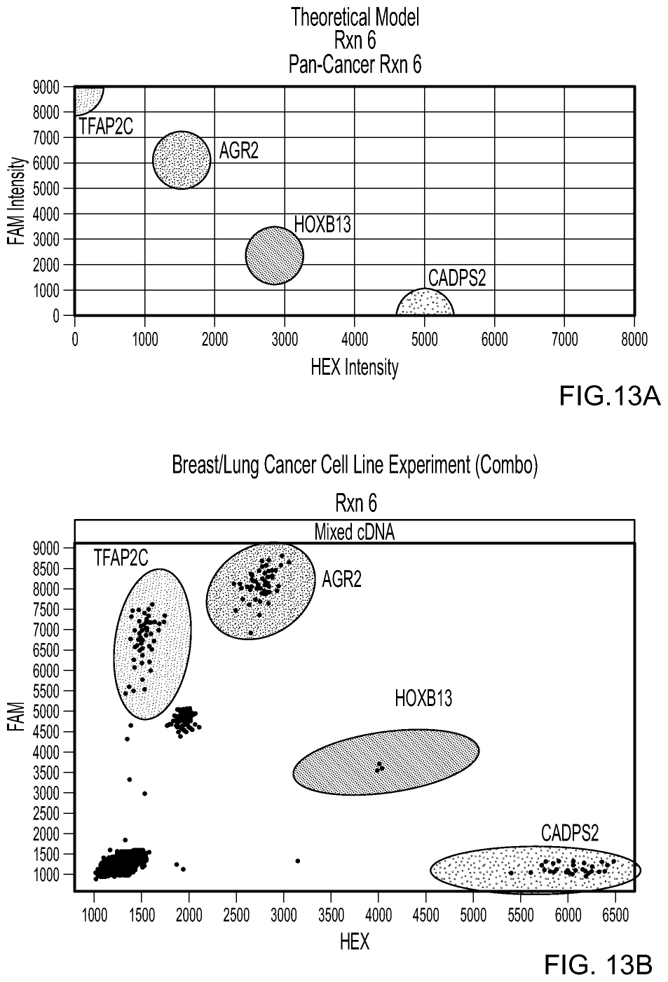

FIGS. 8A-8B (breast and lung cancer theoretical and actual results, Reaction 1), 9A-9B (breast and lung cancer theoretical and actual results, Reaction 2), 10A-10B (same, Reaction 3), 11A-11B (same Reaction 4), 12A-12B (same, Reaction 5), and 13A-13B (same, Reaction 6) illustrate the results when the same approach was use with breast cancer and lung cancer. We can establish a multi-cancer panel that is effective in identifying markers shared by most adenocarcinomas (i.e., grouping breast and lung cancer togher), as 6 reactions (4 gene markers within each reaction for a total of 24 markers), as shown below (theoretical vs validation using spiked cell experiments with both breast and lung cancer cells).

These figures show the results when the same approach of testing multiple gene transcripts in multiplex fashion (4 genes per reaction) was applied to breast cancer. Six different reactions were performed of the same CTC chip product (enabling a total of 24 gene transcripts to be tested independently), with each one having a designated signal position (predicted in upper panel) and observed in spiked cell validation experiments (observed in lower panel).

### Example 5—Target-Specific Pre-Amplification to Improve Detection of Tumor-Specific mRNA

To improve the detection of tumor specific RNAs, a nested PCR strategy was optimized for each of the gene-specific amplifications. To achieve this, cDNA derived from the CTCs was first amplified with gene-specific primers which are situated a few base pairs external to the gene-specific primers used for d-CTC assay. For each gene, two to three primer sets were tested, and the primer set that is compatible with the gene-specific d-CTC assay primer and tests negative in HD blood was chosen for analysis of patient samples.

As described above, the target specific amplification protocol was first tested in cell lines derived from the different cancers. The primer combinations that are specific for tumor cells (and absent in leukocytes) were then tested with a mixture of cancer cell lines mixed into blood and enriched through the CTC-iChip. HD blood processed through the CTC-iChip was used as control. Key to this strategy is the design of the nested PCR conditions to enhance the signal from minute amounts of CTC-derived cDNAs, without increasing the minimal baseline signal from normal blood cells. This selectivity was achieved by careful optimizing of PCR primer sequences and assay conditions, as well as balancing the cycle number for the external and internal PCRs. All conditions are validated first with purified nucleic acids, then with individual tumor cells that are spiked into control blood samples and processed through the CTC-iChip, then with large panels (>10) of different healthy blood donors, and ultimately with patient-derived blood samples from patients who have either metastatic or localized cancers of the prostate, breast, melanoma, liver, lung or pancreas.

### Reagents

DNA Suspension Buffer (10 mM Tris, pH 8.0, 0.1 mM EDTA) (TEKnova, PN T0221)
0.5 EDTA, pH 8.0 (Invitrogen, PN Am9260G)
TaqMan PreAmp Master Mix (Applied Biosystems, PN 4391128)
Nuclease-free Water (TEKnova, PN W330)

### Preparing 10X Specific Target Amplification (STA) Primer Mix

1.) In a DNA-free hood, 0.5 μL of each of 200 μM primer pairs (0.5 μL Forward primer and 0.5 μL Reverse primer) were mixed.
2.) Each primer was diluted in 1X DNA Suspension Buffer to a final concentration of 500 nM. (Ex: If pooled primer volume equals 8 mL, add 192 mL DNA Suspension Buffer)
3.) The mix was vortexed for 20 seconds and spun down for 30 seconds.
4.) 10X STA Primer Mix can be stored at 4° C. for repeated use for up to six months or stored frozen at −20° C. for long-term usage.

### Preparing STA Reaction Mix

1.) For each well of a 96-well PCR plate, prepare the following mix.

| | Per 9 μL | 96 Samples with |
|---|---|---|
| Component | Sample (μL) | overage (μL) |
| TaqMan ® PreAmp | 7.5 | 780.0 |
| Master Mix | | |
| 10X STA Primer Mix | 1.5 | 156.0 |
| (500 nM) | | |
| 0.5M EDTA, pH 8.0 | 0.075 | 7.8 |
| Total Volume | 9.0 | 943.8 |

2.) 6 μL cDNA was added to 9 μL STA reaction mix
3.) Thermocycling conditions listed below were used with 18 cycles of denaturation and annealing/extension steps rather than 20 cycles. (Note: 18 cycles were used to compare TSA Pre-Amplification protocol to Whole Transcriptome Amplification).

| | | 10 to 18 Cycles | | |
|---|---|---|---|---|
| | Enzyme | | Annealing/ | |
| Condition | Activation | Denaturation | Extension | Hold |
| Temperature | 95° C. | 96° C. | 60° C. | 4° C. |
| Time | 10 minutes | 5 seconds | 4 minutes | Infinity |

1 μl of the pre-amplified product is loaded in each droplet PCR reaction.

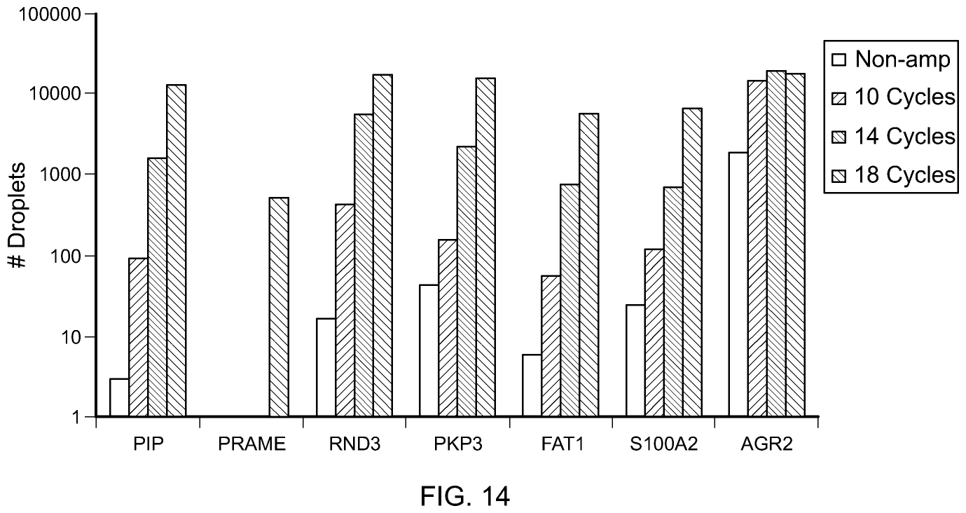

FIG. 14 shows the droplet PCR signal for 7 markers (PIP, PRAME, RND3, PKP3, FAT1, S100A2, and AGR2) from 1 ng of non-amplified cell-line cDNA and from 1 μl of pre-amplified product after 10, 14, and 18 cycles of pre-amplification. Additional cycles of pre-amplification result in signal increase. Of note, PRAME, a marker expressed at very low levels in this cell line is detected only after 18 cycles of pre-amplification, demonstrating the utility of the technique.

### Example 6—Clinical Data and Assay Validation

The assays described herein have been validated using actual patients samples from clinical studies. These include patients with metastatic cancer (lung, breast, prostate and melanoma), as well as patients with localized cancer (prostate). The assays are conducted as described in Examples 2 through 5.

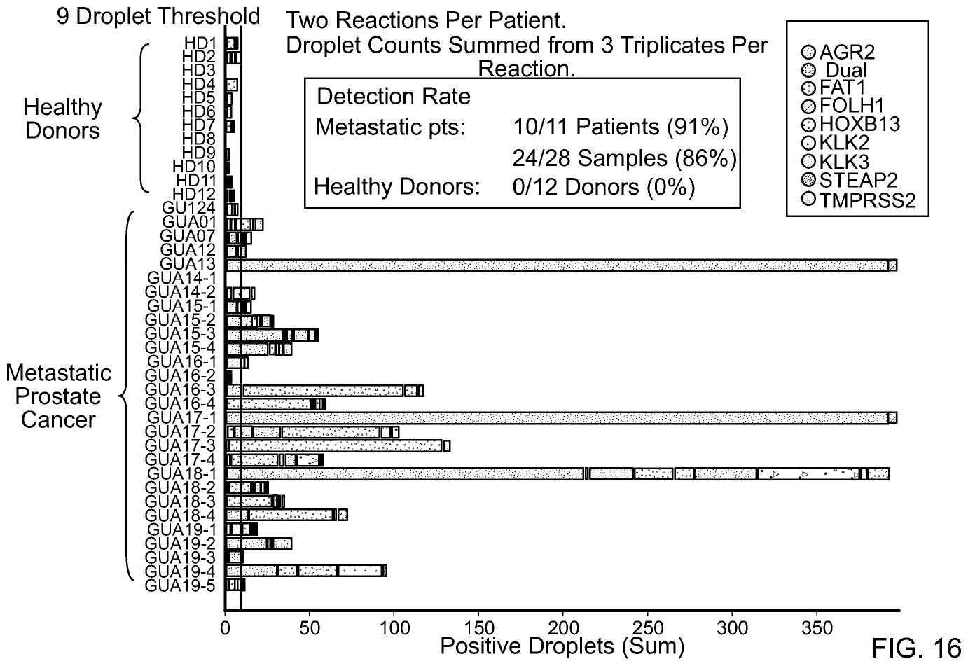

FIGS. 15A, B, and C show a summary of clinical assays from patients with metastatic cancers of the lung (6 patients; FIG. 15A), breast (6 patients; FIG. 15B) and prostate (10 patients; FIG. 15C) showed that virtually all patients have positive signal, whereas healthy controls have none. In this assay, all positive scores were added (cumulative score). However, as described below, the scores can also be broken down by individual genes, as shown in FIG. 16.

FIG. 16 illustrates the cumulative analysis of data from multiple probes, and shows a positive signal in 10/11 metastatic prostate cancer patients (91% on a per patient basis) versus 0/12 (0%) of healthy controls. On a per sample basis, 24 of 28 samples had a positive signal, indicating an 86% detection rate. In addition, some individual markers were also fairly effective, e.g., AGR2 (9/10 detection for metastatic cancer, and 0/3 for localize cancer), TMPRSS2 (5/10 and 1/3), KLK2 (6/10 and 0/3), STEAP2 (1/10 and 1/3), FAT1 (2/10 and 1/3), and FOLH1 (3/10 and 1/3)

As illustrated above, one can also break down the individual gene markers for independent validation and quantitation, using the multiplex fluorescence color scheme described above. In this example below, a patient with metastatic prostate cancer had multiple positive markers, a patient with localized prostate cancer has a smaller number of positive scores within fewer markers, and a healthy control is negative for all markers.

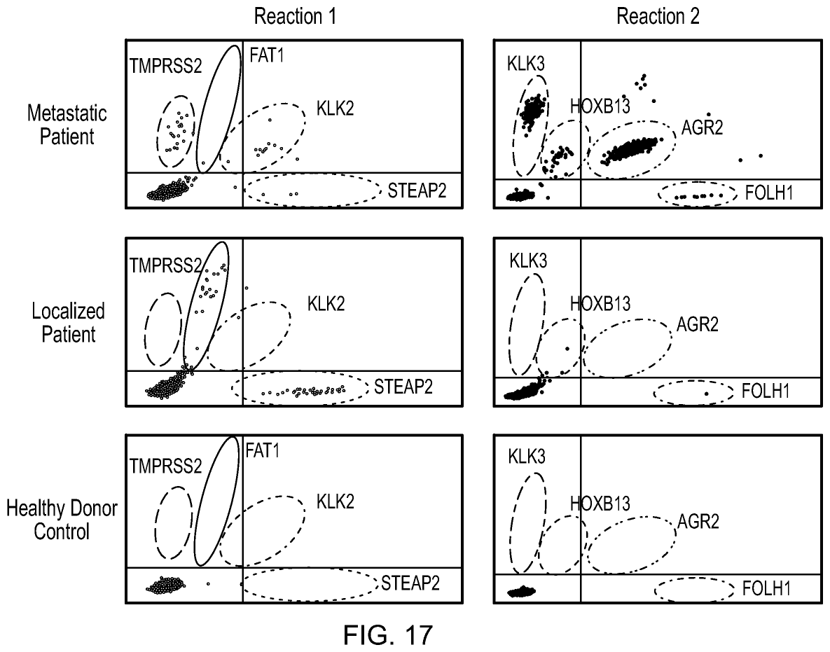

FIG. 17 shows clinical data from three representative patient samples. In two separate reactions with four gene transcripts each (8 probes total), a blood sample from a patient with metastatic prostate cancer showed multiple signals (all probes are positive to various degrees). In contrast, a blood sample from a patient with localized (curable) prostate cancer showed weaker (but clearly detectable) signal. Whereas probes 1 (TMPRSS2), 5 (KLK3), 6 (HOXB13), 7 (AGR2) had the strongest signal in the metastatic cancer patient, probes 2 (FAT1) and 4 (STEAP2) were most positive in the localized cancer patient. This result clearly illustrates the heterogeneity in signal among cancer cells in the blood and the importance of dissecting the differential signals within the assay. Blood from a HD control (processed identically to the cancer patient samples) had a complete absence of signal.

### Example 7—Measurement of Signaling Pathways within CTCs

In addition to providing a digital (quantitative) measure of CTCs present within a blood sample, our d-CTC assay also allowed analysis of specific signaling pathways that are unique to the tumor cells in the blood. For instance, a subset of prostate lineage-specific genes were driven by androgen signaling (such as PSA), while another subset was repressed by androgen signaling (such as PSMA). By analyzing these genes together, we can ascertain the status of androgen signaling within CTCs. Defining the total number of CTC signal in the blood, simultaneously with information about the effectiveness of the therapeutic agent in targeting and shutting off the critical pathway is important for therapeutic monitoring.

We have illustrated this concept in prostate cancer, where the anti-androgenic agent abiratorone is effective in suppressing cancer progression, particularly in tumors that are still dependent on the androgen pathway. Below, we showed the results of a patient with “Castrate Resistant Prostate Cancer (CRPC)” who is no longer responding to first line leuprolide and was treated with abiratorone. The androgen response markers (green) were initially suppressed by the therapy as it shows initial efficacy, but subsequently returned as the tumor becomes resistant and the patient experiences disease progression on this drug.

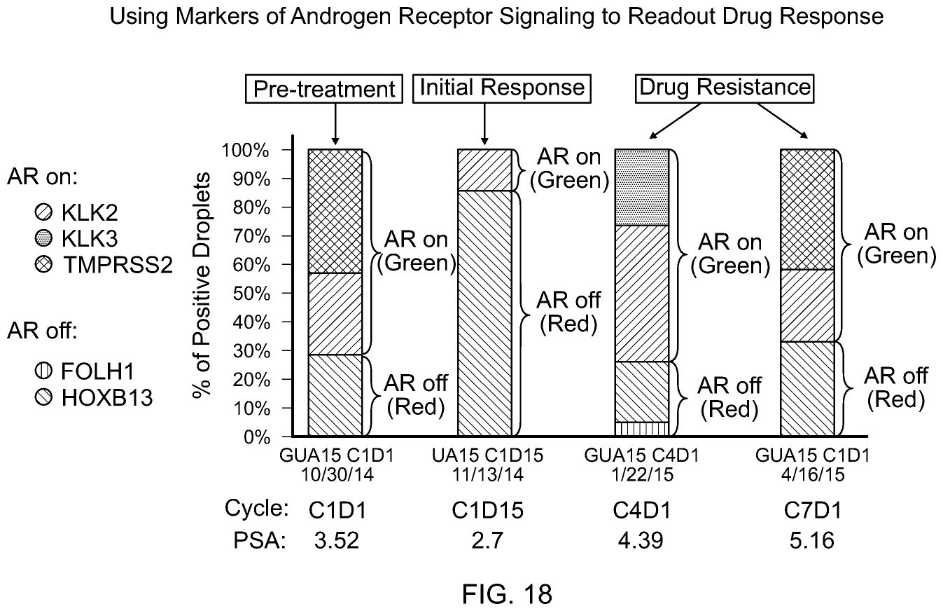

FIG. 18 provides the results of a clinical study of a patient with metastatic prostate cancer. The subset of signals from “androgen receptor-induced genes (AR-On)” is shown in green at the top of the bars in this bar graph, while the subset of signals from “androgen-repressed genes (AR-Off) is shown in red at the bottom of each bar. As the patient is treated with the androgen pathway inhibitor abiratorone (e.g., ZYTIGA® (abiraterone acetate), the AR-On signal is greatly reduced, indicating effective suppression of the androgen pathway within cancer cells in the blood. By cycle 4 of drug treatment, however, the androgen pathway appears to be reactivated in cancer cells (increasing green signal), indicative of drug resistance. Serum PSA measurements taken at these time points are consistent with failure of drug treatment.

### Example 8—Non-Specific Pre-Amplification to Improve Detection of Tumor-Specific mRNA

Similar to Example 5, non-specific whole transcriptome amplification (WTA) can be used to increase the detection rate of CTC-specific transcripts. This method relies on the use of random primers that amplify not only the targets of interest but all messages found in the product. In this example, the SMARTer™ Ultra Low RNA kit protocol (Clontech) was used as described below:

Transfer RNA to PCR tubes or plate
1) Add 1 uL of 1:50,000 diluted ERCC Spike-In Mix 1 to each sample
2) Bring the volume of each sample up to 10 uL
3) Add 1 uL of 3′ SMART CDS Primer IIA to each sample
4) Run “72 C” thermocycler program:
72° C. 3 min
4° C. forever
   First Strand cDNA Master Mix (FSM):
1x 4 uL 5x First-Strand Buffer
0.5 uL DTT
1 uL dNTP Mix
1 uL SMARTer IIA Oligonucleotide
0.5 uL RNase Inhibitor
2 uL SMARTScribe RT
9 uL per sample
5) Prepare the 10% excess FSM for your sample number, then add 9 uL of FSM to each sample and pipet to mix
6) Run “cDNA” thermocycler program:
42° C. 90 min
70° C. 10 min
4° C. forever

### Second Strand Synthesis and Amplification (SSM):

1x 25 uL 2x SeqAmp PCR Buffer
1 uL Primer IIA—v3
1 uL SeqAmp DNA Polymerase
3 uL Nuclease-free water 30 uL per sample
7) Prepare the 10% excess SSM for your sample number, then add 30 uL of SSM to each sample and pipet to mix
8) Run “PCR” thermocycler program:
95° C. 1 min
X cycles
98° C. 10 sec
65° C. 30 sec
68° C. 3 min
72° C. 10 min
4° C. forever
   The number of cycles can be adjusted depending on RNA input (e.g., 18 cycles for single cells or 9 cycles for 10 ng of RNA input). In addition, the 4 degree stopping point is overnight.

### Solid Phase Reversible Immobilization (SPRI) Purification:

Transfer PCR product to lo-bind 1.5 mL Eppendorfs and label a second set of tubes with sample IDs; run the SPRI protocol at RT until the final elution
9) Incubate AMPureTM XP beads [4 deg] at RT for at least 30 minutes
10) Ensure that a sufficient amount of Elution Buffer is thawed and at RT
11) Make 80% ethanol (at least 400 uL per sample)
12) Vortex beads well before adding 50 uL of beads to each sample, pipetting up and down 5-10 times to mix well. Note: When pipetting beads, it's advisable to use RPT tips for better control of the volumes added and less residual bead binding in the tips
13) Incubate samples at RT for 5 minutes
14) Place samples on the magnet and let sit for 5 minutes
15) Pipet out the supernatant (˜95 uL) without disturbing the beads
(check for brown color in the pipet tip and put back in tube if there's a significant amount of bead loss)
16) Wash twice with 200 uL of 80% ethanol—do not mix or disturb the bead pellet. Simply submerge the bead pellet in the ethanol for 30 seconds and then remove the ethanol. Try not to let the bead pellet dry between ethanol washes.

17) Air-dry the samples on the magnetic rack until the bead pellets are no longer shiny but before they crack. Pipet off any residual ethanol that pools at the bottom while drying (Note: The drying time can vary greatly depending on the DNA concentration after amplification). Single-cell level RNA inputs generally take 3-5 minutes to dry, while other IFD product samples have taken up to an hour.
18) Elute pellets in 17 uL of Elution Buffer as they begin to crack. Remove a sample from the magnet and pipet the buffer over the pellet repeatedly until all of the beads are in solution; then pipet mix to fully resuspend the beads (this will work to varying degrees for each sample). Try not to mix too vigorously as this creates many bubbles, which tends to decrease the attainable elution volume.
19) Let the resuspended samples incubate at RT for at least 2 minutes, then quick spin all of the samples.
20) Put the samples back on the magnetic rack for 5 minutes.
21) Pipet off—15 uL of your eluted amplified cDNA and check for beads in the pipette tip. If beads are present, pipet the solution back over the bead pellet and let sit for ˜1 minute before attempting another elution. Otherwise, store in a new lo-bind 1.5-mL Eppendorf, PCR tube, or 96-well PCR plate. Note: If you are repeatedly getting beads in the elution product, the only solution may be to decrease your aspiration volume to 14 uL or lower.

This whole transcriptome amplification (WTA) approach was first tested in cell lines derived from different cancers. FIGS. 19A and 19B show three different replicates of SMARTer-preamplified cDNA (18 cycles) from a liver cancer cell line (HEPG2) analyzed with 12 probes from the liver cancer panel. As shown in FIG. 19A, while the amplification efficiency for each target region is different, it is consistent among the three replicates (WTA1, WTA2, WTA3), demonstrating the reproducibility of this approach. As shown in FIG. 19B, these methods using 18 cycles of SMARTer pre-amplification provide an increase in signal of approximately four orders of magnitude (10⁸ vs 10⁴), providing a great boost in detection.

### Eample 9—Multiplexed vs. Individual Marker Assays for Liver Cancer

For each sample, 10-20 mL of blood was collected from each patient. Blood was processed within 3 hours of arrival on a CTC-iCHIP running in negative depletion mode. RNA was extracted from the product using a Qiagen RNeasy™ plus Micro kit, and 5 uL of the available 17 uL amplified using ClonTech's v3 SMARTer™ whole-transcriptome amplification (WTA) strategy. 1% of the WTA product was then loaded into each well of a digital PCR plate, and 500 nM Taqman™ primer/probe combinations used to determine the transcript concentration for each gene of interest. Transcript counts were normalized to blood volume and compared between HCC, HD, and CLD patients. HCC patients are defined as biopsy-confirmed non-resected hepatocellular carcinoma, CLD patients are patients with liver disease of varying etiologies (alcohol-mediated, HBV, HCV) who have negative ultrasound/MRI. HD are healthy donors external to the lab who donate 10-20 mL of blood.

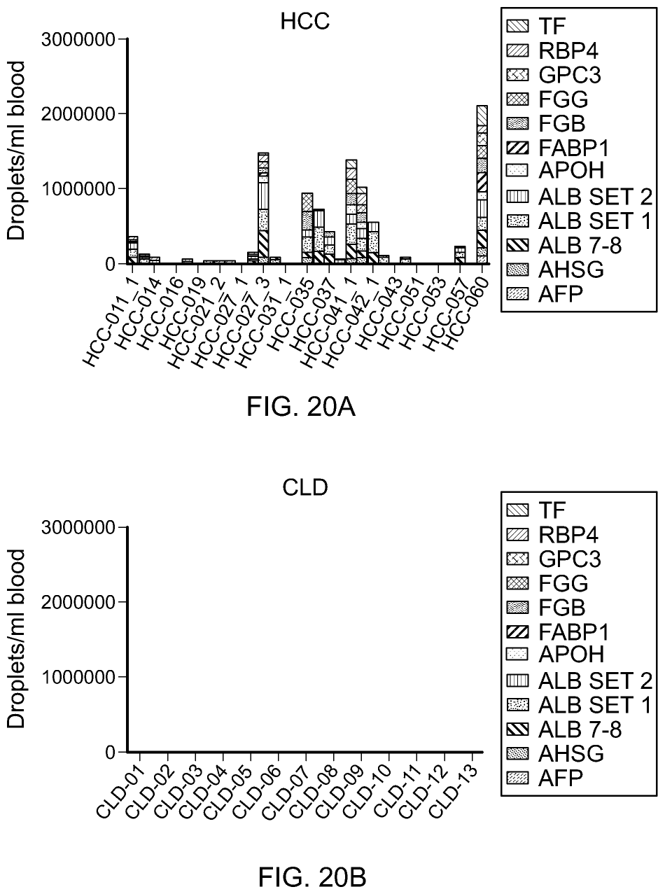

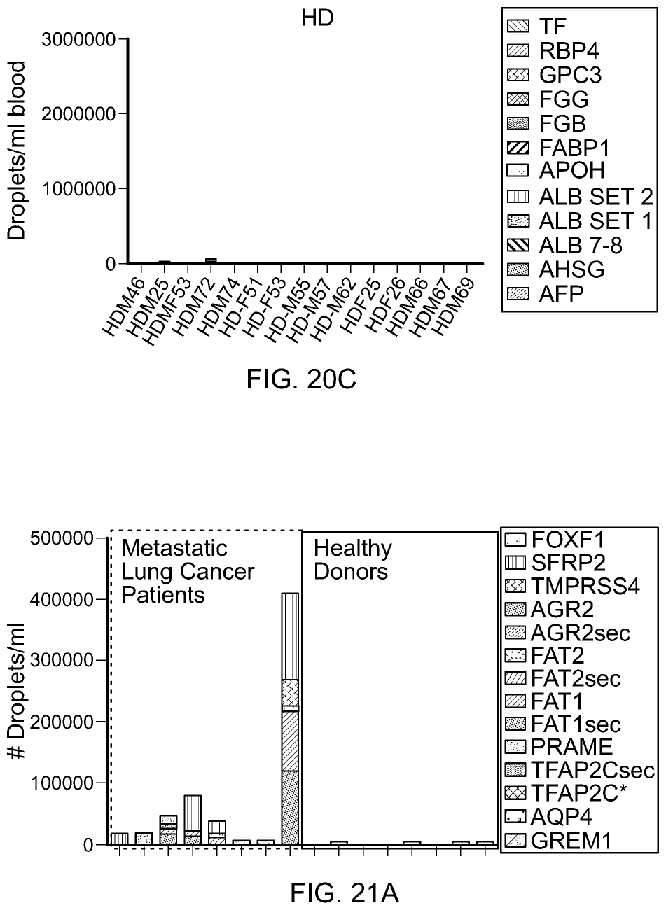

FIGS. 20A to 20C show the total droplet numbers in 21 hepatocellular carcinoma (HCC) patients (FIG. 20A), 13 chronic liver disease (CLD) patients (FIG. 20B) and 15 healthy donors (HDs) (FIG. 20C). HCC patients show higher number of droplets compared to both CLDs and HDs, suggesting that the panel is very clean in the high risk CLD group and can be used to screen those patient for the development of liver cancer. This is an important result given the low specificity of screening methods c=for liver cancer currently available in the clinic. Among CLD patients the American Association of Liver Disease recommends ultrasound (US) every 6 months, with a detailed algorithm dependent on the size of liver lesion detected. A prospective combined AFP gene marker-ultrasound screening in China demonstrated a 37% mortality benefit for those who were screened compared to those who were not, even when the screened population only maintained a compliance rate of 60%.

The sensitivity and specificity of each assay are dependent on the threshold values chosen to define “diseased” vs. “non-diseased,” but using 20 ug/L, the AFP gene marker has a sensitivity between 50-80% and a specificity between 80-90%. In a study using 20 ng/ml as the cut-off point, the sensitivity rose to 78.9%, although the specificity declined to 78.1% (Taketa, Alpha-fetoprotein, J. Med. Technol., 1989;33:1380). On the other hand, the overall detection rate of the present assay was 76% when taking into account the clinical history of the patients and correcting for the ones that received curative resection or liver transplant with 100% specificity.

In addition, while all 11 markers of the liver cancer assay used herein contributed to the 76% sensitivity, the top 5 markers (AHSG, ALB, APOH, FGB and FGG) by themselves have 70% sensitivity, while the top 3 markers alone (ALB, FGB, FGG) result in 67% sensitivity. ALB alone detected 56% of the cases.

### Example 10—Multiplexed vs. Individual Marker Assays for Lung Cancer

Blood samples from 8 metastatic lung patients and 8 healthy donors were processed through the CTC-chip as previously described. Samples were spun down, treated with RNAlater™ and stored at −80 C. RNA was purified and cDNA was synthesized as described. STA was performed on each sample using 6 μl cDNA and the nested primers corresponding to the probes listed in the figure. 1 μl of STA product was loaded per each droplet PCR reaction.

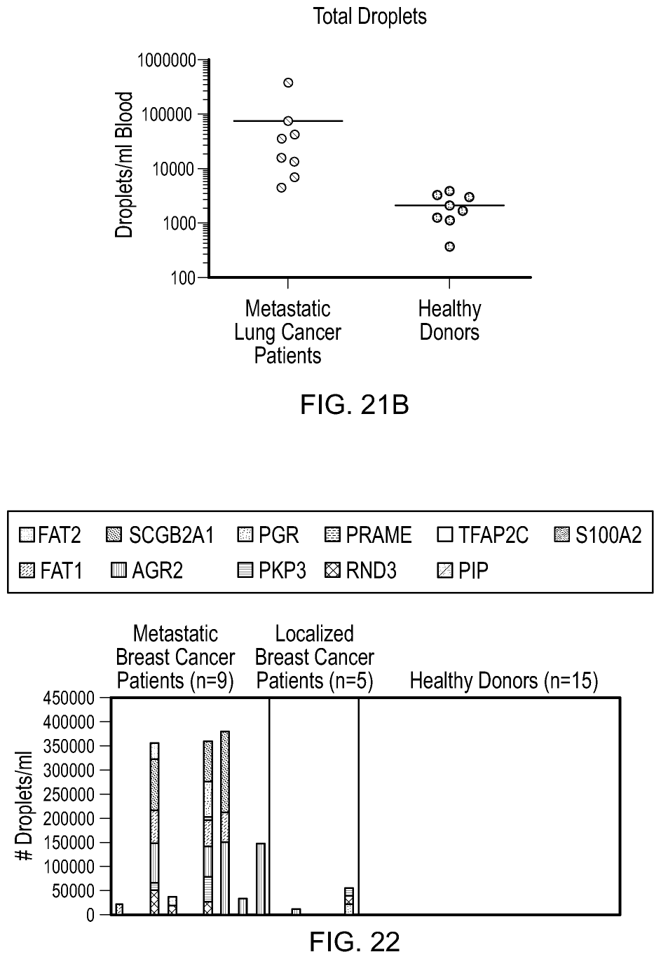

Droplet numbers were normalized to blood volume. As shown in FIG. 21A and 21B, the multiplexed lung gene marker panel was able to detect 100% (8/8) metastatic lung cancer patient samples above the background of the 8 healthy donors. The sensitivity of each marker of the lung panel was also determined and the results show that SFRP had a detection rate of 8/8, FAT1 Probe 2 had a detection rate of 7/8,TMPRSS4 had a detection rate of 6/8, FOXF1 and ARG2, Probe 2 had a detection rate of 5/8, FAT1 had a detection rate of 4/8, FAT2 and AGR2 had a detection rate of 3/8, and FAT2, Probe 2 had a detection rate of 2/8.

Assays for SERPINA3 and SFRP2 indicated that SFRP2 is effective for both lung and breast cancer detection, whereas the former seems more specific for breast cancer detection, but also detects some lung cancer samples.

### Example 11—Multiplexed vs. Individual Marker Assays for Breast Cancer

Blood samples from 9 metastatic breast cancer patient, 5 localized breast cancer patients, and 15 healthy donors were processed though the CTC-Chip. Products were pelleted, treated with RNAlater™ and stored at −80 C. RNA and cDNA from each sample were prepared as previously described. 6 μl cDNA from each sample was STA amplified using nested primers corresponding to the probes listed in FIG. 22 (FAT2, SCGB2A1, PGR, PRAME, TFAP2C, S100A2, FAT1, AGR2, PKP3, RND3, and PIP). Droplet numbers were normalized to blood volumes and the highest healthy donor value for each marker was subtracted from the patient sample values.

FIG. 22 shows the above-background signal for each patient. These methods detected 7/9 (78%) of metastatic samples and 2/5 (40%) of localized samples. The sensitivity of each marker alone varied from 1/14 to 6/14, with the two most relevant markers being AGR2 (6/14) and FAT1 (5/14), and the next four most relevant markers being RND3, PKP3, PRAME, and SCGB2A1 (3/14 each).

### Example 12—AVR7 Detection in Metastatic Breast Cancer

Blood samples from 10 metastatic breast cancer patient and 7 healthy donors were processed though the CTC-Chip. Products were pelleted, treated with RNAlater™ and stored at −80 C. RNA and cDNA from each sample were prepared as previously described. 6 μl of non-amplified cDNA were loaded into each droplet PCR reaction. The samples were analyzed with probes against the v7 isoform of the lo androgen receptor (ARv7, sequence in Table 1). Droplet number was normalized to blood volume.

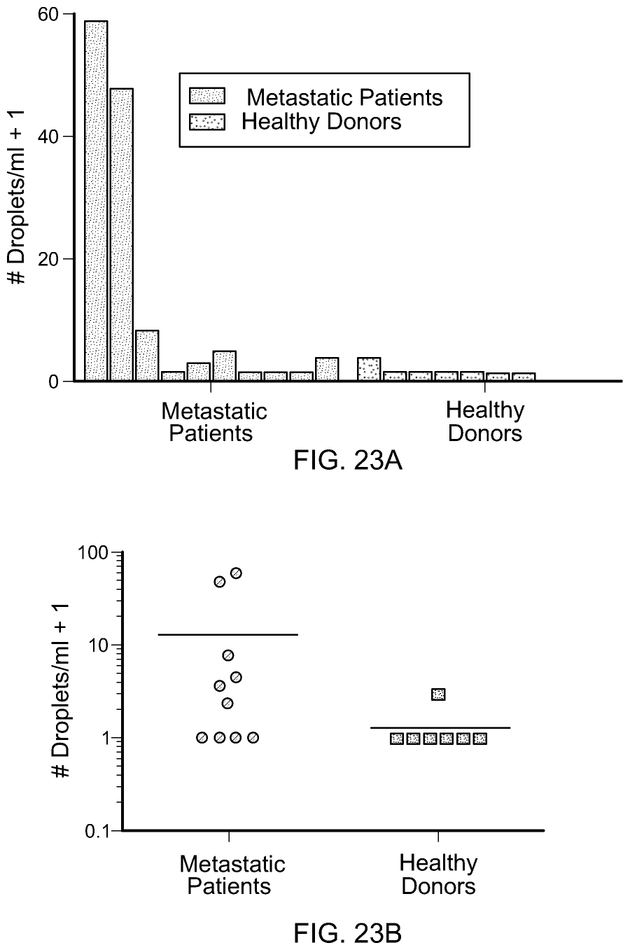

As shown in FIG. 23A, ARv7 was detected in 5/10 patients (50%) at above background (HD) levels, demonstrating that the assay is successful at detecting ARv7 from liquid biopsy. One of the patients had a triple negative breast cancer, suggesting utility of ARv7 as a marker even in the triple negative breast cancer (TNBC) context (e.g., patients who do not express genes for any of the three most common breast cancer markers, the estrogen receptor (ER), HER2/neu, and the progesterone receptor (PR) marker).

### Example 13—Multiplexed vs. Individual Marker Assays for Melanoma

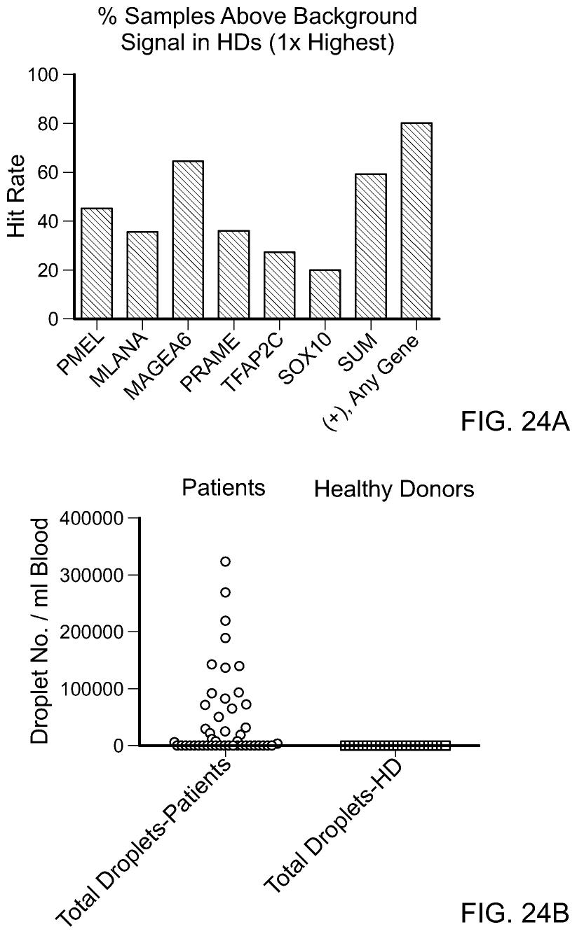

Blood samples from 34 metastatic or unresectable melanoma patients, each with multiple draw points (total draw points: 182), and 15 healthy donors were processed though the CTC-Chip. Products were pelleted, treated with RNAlater™ and flash frozen at −80 C. RNA and cDNA from each sample were prepared as previously described. 12 μl cDNA from each sample was amplified by specific target amplification (10 cycles) using nested primers corresponding to the probes listed along the bottom of the graph in FIG. 24A (individual markers PMEL, MLANA, MAGEA6, PRAME, TFAP2C, and SOX10)). Droplet numbers were normalized to blood volumes. FIG. 24B shows a dot plot distribution of droplet signals detected in melanoma patients as compared to healthy donors. The detection sensitivity was 81% for all patient draw points (a patient draw is scored positive if any 1 of 6 markers shows droplet signals above the highest background signal in HD for that particular marker). Of the individual markers, PMEL and MLANA showed the highest detection rate.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for analyzing circulating tumor cells (CTCs) in a blood sample from a subject, the method comprising:
isolating circulating tumor cells (CTCs) from the blood sample;
isolating ribonucleic acid (RNA) from the CTCs;
generating cDNA molecules in solution from the isolated RNA;
encapsulating cDNA molecules into individual droplets;
amplifying cDNA within each of the individual droplets in the presence of one or more reporter groups configured to bind specifically to cDNA corresponding to tumor lineage-specific RNA from a specific type of tissue that is the source of the CTCs and not to bind to cDNA from normal cells in the blood;
detecting droplets that contain the reporter groups as an indicator of the presence of amplified cDNA molecules from CTCs in the droplets; and
analyzing cDNA molecules from CTCs in the detected droplet;
wherein amplifying cDNA molecules within each of the individual droplets comprises conducting PCR in each droplet, wherein at least one primer set for each type of cancer is used for amplifying the cDNA molecules within each of the droplets, wherein each primer set corresponds to a selected cancer gene,
wherein the selected cancer genes include genes selective for one or more of lung cancer, liver cancer, melanoma, and pancreatic cancer,
wherein the lung cancer-selective genes include one or more of AQP4, GREM1, TFAP2C, PRAME, SFRP2, FAT1, TMPRSS4, FOXF1, ARG2, and FAT2, and wherein the primer sets corresponding to the lung cancer-selective genes are:
AQP4 (primer 1 SEQ ID NO:200, primer 2 SEQ ID NO:199),
GREM1 (primer 1 SEQ ID NO:206, primer 2 SEQ ID NO:205),
TFAP2C (primer 1 SEQ ID NO:131, primer 2 SEQ ID NO:130),
PRAME (primer 1 SEQ ID NO:149, primer 2 SEQ ID NO:148),
SFRP2 (primer 1 SEQ ID NO:194, primer 2 SEQ ID NO:193),
FAT1 (primer 1 SEQ ID NO:23, primer 2 SEQ ID NO:22),
TMPRSS4 (primer 1 SEQ ID NO:203, primer 2 SEQ ID NO:202),
FOXF1 (primer 1 SEQ ID NO:209, primer 2 SEQ ID NO:208),
AGR2 (primer 1 SEQ ID NO:2, primer 2 SEQ ID NO:1), and
FAT2 (primer 1 SEQ ID NO:26, primer 2 SEQ ID NO:25),
wherein the liver cancer-selective genes include one or more of TF, RBP4, GPC3, AFP, AHSG, ALB, FABP1, APOH, FGB, and FGG, and wherein the primer sets corresponding to the liver cancer-selective genes are:
TF (primer 1 SEQ ID NO:251, primer 2 SEQ ID NO:250),
RBP4 (primer 1 SEQ ID NO:248, primer 2 SEQ ID NO:247),
GPC3 (primer 1 SEQ ID NO:137, primer 2 SEQ ID NO:136),
AFP (primer 1 SEQ ID NO:122, primer 2 SEQ ID NO:121),
AHSG (primer 1 SEQ ID NO:152, primer 2 SEQ ID NO:151),
ALB (primer 1 SEQ ID NO:140, primer 2 SEQ ID NO:139),
FABP1 (primer 1 SEQ ID NO:236, primer 2 SEQ ID NO:235),
APOH (primer 1 SEQ ID NO:233, primer 2 SEQ ID NO:232),
FGB (primer 1 SEQ ID NO:239, primer 2 SEQ ID NO:238), and
FGG (primer 1 SEQ ID NO:242, primer 2 SEQ ID NO:241);
wherein the melanoma-selective genes include one or more of PMEL, MLANA, MAGEA6, PRAME, TFAP2C, and SOX10, and wherein the primer set corresponding to the melanoma-selective genes are:
PMEL (primer 1 SEQ ID NO:68, primer 2 SEQ ID NO:67),
MLANA (primer 1 SEQ ID NO:50, primer 2 SEQ ID NO:49),
MAGEA6 (primer 1 SEQ ID NO:44, primer 2 SEQ ID NO:43),
PRAME (primer 1 SEQ ID NO:149, primer 2 SEQ ID NO:148),
TFAP2C (primer 1 SEQ ID NO:131, primer 2 SEQ ID NO:130), and
SOX10 (primer 1 SEQ ID NO:89, primer 2 SEQ ID NO:88); and
wherein the pancreatic cancer-selective genes include one or more of ALDH1A3, CDH11, EGFR, FAT1, MET, PKP3, RND3, S100A2, and STEAP2, and
wherein the primer set corresponding to the pancreatic cancer-selective genes are:
ALDH1A3 (primer 1 SEQ ID NO:5, primer 2 SEQ ID NO:4),
CDH11 (primer 1 SEQ ID NO:11, primer 2 SEQ ID NO:11),
EGFR (primer 1 SEQ ID NO:20, primer 2 SEQ ID NO:19),
FAT1 (primer 1 SEQ ID NO:23, primer 2 SEQ ID NO:22),
MET (primer 1 SEQ ID NO:47, primer 2 SEQ ID NO:46),
PKP3 (primer 1 SEQ ID NO:65, primer 2 SEQ ID NO:64),
RND3 (primer 1 SEQ ID NO:77, primer 2 SEQ ID NO:76),
S100A2 (primer 1 SEQ ID NO:80, primer 2 SEQ ID NO:79), and
STEAP2 (primer 1 SEQ ID NO:125, primer 2 SEQ ID NO:124).

2. The method of claim 1, further comprising reducing a volume of the product before isolating RNA.

3. The method of claim 1, further comprising removing contaminants from the solution containing the cDNA molecules before encapsulating the cDNA molecules.

4. The method of claim 1, wherein generating the cDNA molecules from the isolated RNA comprises conducting reverse transcription (RT) polymerase chain reaction (PCR) of the isolated RNA.

5. The method of claim 1, wherein amplifying cDNA within droplets comprises conducting PCR in a plurality of the droplets.

6. The method of claim 1, wherein encapsulating individual cDNA further comprises encapsulating PCR reagents in individual droplets with the cDNA and forming at least 1000 droplets of a non-aqueous liquid.

7. The method of claim 1, wherein the one or more reporter groups comprise a fluorescent label.

8. The method of claim 3, wherein removing contaminants from the solution containing the cDNA molecules comprises the use of Solid Phase Reversible Immobilization (SPRI).

9. The method of claim 8, wherein the SPRI comprises
immobilizing cDNA in the solution with magnetic beads that are configured to specifically bind to the cDNA;
removing contaminants from the solution; and
eluting purified cDNA.

10. The method of claim 6, wherein the non-aqueous liquid comprises one or more fluorocarbons, hydrofluorocarbons, mineral oils, silicone oils, and hydrocarbon oils.

11. The method of claim 1, wherein the CTCs arise from metastatic or primary/localized cancers.

12. The method of claim 1, wherein analyzing the CTCs in the detected droplets comprises monitoring CTCs from blood samples taken over time from a patient with a known cancer, and testing, imaging, or both testing and imaging the CTCs to provide a prognosis for the patient.

13. The method of claim 1, wherein analyzing the CTCs in the detected droplets comprises testing, imaging, or testing and imaging the CTCs to provide an indication of a response by the CTCs to a therapeutic intervention.

14. The method of claim 1, wherein analyzing the CTCs in the detected droplets comprises determining a number or level of CTCs per unit volume of a blood sample from a patient to provide a measure of tumor burden in the patient.

15. The method of claim 14, further comprising using the measure of tumor burden in the patient to select a therapy.

16. The method of claim 14, further comprising determining the measure of tumor burden in the patient at a second time point to monitor the tumor burden over time.

17. The method of claim 1, wherein the cDNA is pre-amplified prior to amplifying the cDNA within the droplets.

18. The method of claim 17, wherein the cDNA is pre-amplified using nested primers corresponding to one or more primers that relate to one or more cancer-selective genes for lung cancer, liver cancer, melanoma, or pancreatic cancer.

19. The method of claim 18, wherein the cDNA is pre-amplified using non-specific whole transcriptome amplification (WTA).

20. A composition comprising at least one primer set for amplifying cDNA molecules derived from circulating tumor cells (CTCs),
wherein each primer set corresponds to a selected cancer gene,
wherein the selected cancer genes include genes selective for one or more of lung cancer, liver cancer, melanoma, and pancreatic cancer,
wherein the lung cancer-selective genes include one or more of AQP4, GREM1, TFAP2C, PRAME, SFRP2, FAT1, TMPRSS4, FOXF1, ARG2, and FAT2, and wherein the primer sets corresponding to the lung cancer-selective genes are:
AQP4 (primer 1 SEQ ID NO:200, primer 2 SEQ ID NO:199),
GREM1 (primer 1 SEQ ID NO:206, primer 2 SEQ ID NO:205),
TFAP2C (primer 1 SEQ ID NO:131, primer 2 SEQ ID NO:130),
PRAME (primer 1 SEQ ID NO:149, primer 2 SEQ ID NO:148),
SFRP2 (primer 1 SEQ ID NO:194, primer 2 SEQ ID NO:193),
FAT1 (primer 1 SEQ ID NO:23, primer 2 SEQ ID NO:22),
TMPRSS4 (primer 1 SEQ ID NO:203, primer 2 SEQ ID NO:202),
FOXF1 (primer 1 SEQ ID NO:209, primer 2 SEQ ID NO:208),
AGR2 (primer 1 SEQ ID NO:2, primer 2 SEQ ID NO:1), and
FAT2 (primer 1 SEQ ID NO:26, primer 2 SEQ ID NO:25),
wherein the liver cancer-selective genes include one or more of TF, RBP4, GPC3, AFP, AHSG, ALB, FABP1, APOH, FGB, and FGG, and wherein the primer sets corresponding to the liver cancer-selective genes are:
TF (primer 1 SEQ ID NO:251, primer 2 SEQ ID NO:250),
RBP4 (primer 1 SEQ ID NO:248, primer 2 SEQ ID NO:247),
GPC3 (primer 1 SEQ ID NO:137, primer 2 SEQ ID NO:136),
AFP (primer 1 SEQ ID NO:122, primer 2 SEQ ID NO:121),
AHSG (primer 1 SEQ ID NO:152, primer 2 SEQ ID NO:151),
ALB (primer 1 SEQ ID NO:140, primer 2 SEQ ID NO:139),
FABP1 (primer 1 SEQ ID NO:236, primer 2 SEQ ID NO:235),
APOH (primer 1 SEQ ID NO:233, primer 2 SEQ ID NO:232),
FGB (primer 1 SEQ ID NO:239, primer 2 SEQ ID NO:238), and
FGG (primer 1 SEQ ID NO:242, primer 2 SEQ ID NO:241);
wherein the melanoma-selective genes include one or more of PMEL, MLANA, MAGEA6, PRAME, TFAP2C, and SOX10, and wherein the primer set corresponding to the melanoma-selective genes are:
PMEL (primer 1 SEQ ID NO:68, primer 2 SEQ ID NO:67),
MLANA (primer 1 SEQ ID NO:50, primer 2 SEQ ID NO:49),
MAGEA6 (primer 1 SEQ ID NO:44, primer 2 SEQ ID NO:43),
PRAME (primer 1 SEQ ID NO:149, primer 2 SEQ ID NO:148),
TFAP2C (primer 1 SEQ ID NO:131, primer 2 SEQ ID NO:130), and
SOX10 (primer 1 SEQ ID NO:89, primer 2 SEQ ID NO:88); and
wherein the pancreatic cancer-selective genes include one or more of ALDH1A3, CDH11, EGFR, FAT1, MET, PKP3, RND3, S100A2, and STEAP2, and wherein the primer set corresponding to the pancreatic cancer-selective genes are:
ALDH1A3 (primer 1 SEQ ID NO:5, primer 2 SEQ ID NO:4),
CDH11 (primer 1 SEQ ID NO:11, primer 2 SEQ ID NO:11),
EGFR (primer 1 SEQ ID NO:20, primer 2 SEQ ID NO:19),
FAT1 (primer 1 SEQ ID NO:23, primer 2 SEQ ID NO:22),
MET (primer 1 SEQ ID NO:47, primer 2 SEQ ID NO:46),
PKP3 (primer 1 SEQ ID NO:65, primer 2 SEQ ID NO:64),
RND3 (primer 1 SEQ ID NO:77, primer 2 SEQ ID NO:76),
S100A2 (primer 1 SEQ ID NO:80, primer 2 SEQ ID NO:79), and
STEAP2 (primer 1 SEQ ID NO:125, primer 2 SEQ ID NO:124).
